SEQUENCE LISTING (6 sequences)

SEQ ID NO: 1 - DNA, 16 bp, Homo sapiens
  source 1..16: mol_type = genomic DNA
gatattcgtacggatt

SEQ ID NO: 2 - DNA, 16 bp, Homo sapiens
  source 1..16: mol_type = genomic DNA
gatgttcgtactgaat

SEQ ID NO: 3 - DNA, 16 bp, Homo sapiens
  source 1..16: mol_type = genomic DNA
gatattcgtacggatt

SEQ ID NO: 4 - DNA, 16 bp, Homo sapiens
  source 1..16: mol_type = genomic DNA
gatattcgtacggaat

SEQ ID NO: 5 - DNA, 16 bp, Homo sapiens
  source 1..16: mol_type = genomic DNA
gatgttcgtactgaat

SEQ ID NO: 6 - DNA, 16 bp, Homo sapiens
  source 1..16: mol_type = genomic DNA
gatgttcgtactgaat

(19) United States
(12) Patent Application Publication
(10) Pub. No.: US 2026/0279495 A1
(43) Pub. Date: Sep. 17, 2026

(54) METHODS FOR PREDICTING RESPONSE TO PHARMACOLOGICAL AGENTS IN PATIENTS WITH OBESITY

(71) Applicants: Phenomix Sciences Inc., St. Paul, MN (US); Mayo Foundation for Medical Education and Research, Rochester, MN (US)

(72) Inventors: Andres J. Acosta, Rochester, MN (US); Michael L. CAMILLERI, Rochester, MN (US); Lizeth CIFUENTES, Rochester, MN (US); Timothy O'CONNOR, Rochester, MN (US)

(21) Appl. No.: 19/164,141

(22) PCT Filed: Mar. 13, 2024
(86) PCT No.: PCT/US2024/019806

§ 371 Date: Sep. 10, 2025

Related U.S. Application Data
(60) Provisional application No. 63/451,814, filed on Mar. 13, 2023.
(60) Provisional application No. 63/590,387, filed on Oct. 13, 2023.

(51) Int. Cl. G16B 40/00; A61K 38/26; G16B 20/20; G16H 50/30

(52) U.S. Cl. CPC G16B 40/00; A61K 38/26; G16B 20/20; G16H 50/30

(57) ABSTRACT

The present disclosure relates to methods and systems for determining an obesity phenotype in an individual with obesity that utilizes predictive machine learning models and gene risk score calculations. Methods are also provided for selecting responders and non-responders to select pharmacotherapies based on an individual with obesity's determined obesity phenotype.

### CROSS-REFERENCE TO RELATED APPLICATIONS

This application claims the benefit of priority to U.S. Provisional Application No. 63/451,814 filed Mar. 13, 2023, and, U.S. Provisional Application No. 63/590,387 filed Oct. 13, 2023, each of which is hereby incorporated by reference in its entirety for all purposes.

### STATEMENT REGARDING FEDERAL FUNDING

This invention was made with government support under DK067071 and DK114460 awarded by the National Institutes of Health. The government has certain rights in the invention.

### FIELD

The present disclosure relates to methods for ascertaining a subject's obesity phenotype using a multi-omics approach or assessing individuals and populations for genetic variants of interest that relate to a particular phenotypic trait (e.g., obesity) and using polygenic risk scores and machine learning to determine the presence of said phenotypic trait based on said genetic variants of interest. The present disclosure also provides methods for ascertaining a subject's obesity phenotype using a multi-omics approach as well as methods for stratifying subjects with obesity as responders and non-responders to specific pharmacological interventions for treating obesity.

### BACKGROUND

Human genetics drive our propensity to have certain characteristics. These characteristics are often complex and result from differences in several parts of our genome. Now that technologies are available to identify genetic differences between individuals (variants), the challenge becomes identifying which variants are relevant to a particular characteristic, such as susceptibility to a disease. Being able to identify relevant variants and how they contribute to a characteristic enables prediction of, among other things, disease risk. Methods that do this identification and predict the likelihood of a characteristic are Polygenic Risk Scores (PRSs).

Obesity is a chronic and complex disease affecting almost 600 million adults worldwide (see Collaborators GO, N. Engl. J. Med., 377:13-27 (2017). Obesity complexity is related to a multi-factorial imbalance between energy intake and energy expenditure, leading to excess energy storage (Hill, Wyatt, and Peters 2012). Most interventions aim to reduce caloric intake; however, there is considerable heterogeneity in all responses. Furthermore, several processes that favor weight gain affect this energy balance equilibrium after weight loss (Aronne et al. 2021; Christofferson et al. 2022). Consequently, understanding how to control this energy balance in humans is essential for developing successful weight loss interventions.

The stages of appetite have been identified as hunger, satiation, and postprandial satiety (Blundell and Halford 1994). Satiation is the process that controls the meal size and leads to meal termination (Cannon and Washburn 1993). Satiation is the result of volume-dependent signals from the gastrointestinal tract, as well as enteroendocrine hormones released by the interaction of the gut wall with nutrients (Cifuentes and Acosta 2022). It has been defined as a sense of fullness after eating or even symptoms such as nausea and bloating, and it has been quantified by the number of calories ingested to reach fullness (Camilleri 2015; Halawi et al. 2017). Further, abnormal satiation has been identified as one of the identified phenotypes of obesity (Acosta. Camilleri, Shin, Vazquez-Roque, et al. 2015) and patients with obesity and abnormal satiation are more prone to gain weight compared to patients with normal satiation. Finally, these patients' responses to all obesity therapies may differ. For example, patients with abnormal satiation were able to loss more weight with phentermine-topiramate than patients with normal satiation (Acosta, Camilleri, Shin, Vazquez-Roque, et al. 2015).

As a result, it has become critical to generate and utilize methods for swiftly identifying these individuals and ascertain whether or not someone would respond to weight-loss interventions. The present disclosure aims to solve this need by using either polygenic risk scores and machine learning or a multi-omics approach in order to identify an individual's obesity phenotype.

### SUMMARY

In one aspect, provided herein is a computer-implemented method for generating a machine learning assisted gene risk score (ML-GRS) for predicting an obesity phenotype of interest in a subject suffering from obesity, the method comprising: (a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein each gene in the set of genes is known or suspected to play a role in obesity; (b) generating by the computer system, a contribution factor for each SNP located in, around or near a gene from the set of genes from the genetic dataset for a subject from the population of subjects, wherein the contribution factor is a numerical value, vector, matrix, or function of various biological factors that represents the contribution or effect that each SNP has on a specific trait known to be associated with the obesity phenotype of interest; (c) calculating by the computer system, a gene risk score (GRS) for each gene in the set of genes by integrating the contribution factors generated in step (b) for each SNP for each gene from the set of genes and SNP genotype information located in, around, or near each gene in the set of genes, wherein the GRS for each gene represents the cumulative effect of the variation in SNPs located in, around, or near the gene in that particular subject has on the function or regulation of the gene so as to increase or decrease the risk of the subject possessing a specific trait known to be associated with the obesity phenotype of interest; (d) iteratively performing by the computer system, steps (a)-(c) for each additional subject from the population of subjects to generate a set of GRSs for each gene in the set of genes from the population of subjects, wherein each gene has a GRS for each sex; (e) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts if any one subject possesses the specific trait known to be associated with the obesity phenotype of interest by combining gene risk scores and additional biological, psychological, or environmental measurements of the any one subject; (f) training by the computer system, the ML-GRS from step (e) using a classifier model to predict subjects from a training set of samples from a training population of obese subjects as being positive for the specific trait known to be associated with the obesity phenotype of interest, thereby generating a trained ML-GRS; and (g) determining by the computer system, if a test subject is positive for the specific trait known to be associated with the obesity phenotype of interest by: (i) calculating by the computer system the ML-GRS for the test subject using steps (a)-(e) on a sample obtained from the test subject; and (ii) comparing by the computer system, the ML-GRS for the test subject to the trained ML-GRS, wherein the test subject is positive for the specific trait known to be associated with the obesity phenotype of interest if the ML-GRS for the subject is above the sex-specific 75^{th} percentile for the specific trait known to be associated with the obesity phenotype of interest or negative for the specific trait known to be associated with the obesity phenotype of interest if the ML-GRS for the subject is below the sex-specific 75^{th} percentile for the specific trait known to be associated with the obesity phenotype of interest. In some cases, the SNP genotype information comprises risk allele dosage, major allele presence, occurrence of a de novo variant, insertion, deletion, or genome rearrangement. In some cases, each gene in the set of genes is known or suspected to play a role in appetite regulation, energy expenditure, lipid metabolism or adipogenesis. In some cases, the subject is obese if the subject possesses a body mass index (BMI)>30 kg/m² with or without type 2 diabetes. In some cases, the ML-GRS for the obesity phenotype of interest predicts the specific trait known to be associated with the obesity phenotype of interest with a sensitivity and/or specificity is at least 65%. In some cases, the ML-GRS predicts the specific trait known to be associated with the obesity phenotype of interest with an area under the curve (AUC) of at least 0.65, 0.7, 0.75, 0.8, 0.85, 0.9 0.95 or 1. In some cases, the ML-GRS predicts the specific trait known to be associated with the obesity phenotype of interest with positive predictive value of at least 50%. In some cases, the contribution factor is a function or algorithm that utilizes output of a regression analysis or a statistical analysis on the genetic dataset as well as one or more additional parameters, covering regression against one or more traits using one or more types of genetic datasets selected from the group consisting of a genome-wide data microarray chip (GWAS), targeted sequencing (exome or targeted genetic panel), variants detected by qPCR through targeted amplification, whole genome sequencing (WGS), targeted or untargeted sequencing of genomic re-arrangement, deletions, duplications, repeat extensions, and complex genotyping of HLA and CYP genes. In some cases, the one or more additional parameters relate to biological factors regarding genetics, epigenetics, and gene regulation for each SNP. In some cases, the one or more additional parameters are selected from the group consisting of proximity of the SNP to the specific gene, known or theoretical mechanistic role of the SNP metadata regarding the SNP in relationship to the gene, and any combination thereof. In some cases, the known or theoretical mechanistic role of the SNP is selected from the group consisting of synonymous mutation, nonsynonymous mutation, frameshift mutation and nonsense mutation. In some cases, the known or theoretical mechanistic role of the SNP affects the three-dimensional structure of the translated protein binding sites, protein-protein interaction sites and modification sites, wherein the modification sites are selected from the group consisting of phosphorylation, glycosylation and proteolytic site. In some cases, the mechanistic role comprises whether or not the SNP is within an intron, exon, enhancer region or regulatory region of the gene, a cis-regulatory element, a promoter region, a non-coding exonic region, a coding exonic region, intronic region, splice site, transcription factor binding site, epigenetic modification site, at a remote genomic location involved in a three-dimensional chromatin contact with the gene, or a cell-type-specific topological-associated domain that contains the gene. In some cases, the cis-regulatory element is an enhancer or insulator. In some cases, the epigenetic modification site comprises DNA methylation modifications, histone modifications, and/or chromatin accessibility. In some cases, the non-genetic and/or multi-omic data is selected from the group consisting of metabolomic data, proteomic data, peptidomic data, epigenetic data, microbiome data, results from one or more questionnaires and any combination thereof. In some cases, the epigenetic data comprises a presence or absence of DNA modifications selected from the group consisting of DNA methylation modifications, histone modifications and chromatin accessibility. In some cases, the microbiome data is from any site on the subject or the subject's environment. In some cases, the machine learning model in step (e) comprises a forward feature selection, backward feature selection, or random feature sampling algorithm and a random forest, GBM, or linear predictor that iteratively selects a feature with a desired training accuracy using the random forest predictor in multiple rounds until no further training accuracy improvement is detected. In some cases, the GRSs of step (e) are normalized. In some cases, each GSR provides an indication of a role or effect that the SNPs located around or near a specific gene may play on the specific trait known to be associated with the obesity phenotype of interest. In some cases, the SNP is considered to be located around or near a specific gene if it is located within at least 500,000 kilobases (kb) of the specific gene. In some cases, the method further comprises performing a genetic analysis on a sample obtained from the subject suffering from or suspected of suffering from obesity prior to step (a). In some cases, the genetic analysis comprises obtaining sequence reads from the whole or portions of the whole genome of the subject. In some cases, the genetic analysis comprises performing a genotyping method selected from the group consisting of restriction fragment length polymorphism identification (RFLPI), random amplified polymorphic detection (RAPD), amplified fragment length polymorphism detection (AFLPD), polymerase chain reaction (PCR), DNA sequencing, RNA sequencing, allele specific oligonucleotide (ASO) probes, and hybridization to microarrays or beads. In some cases, the method further comprises performing by the computer system, a regression analysis and/or statistical analysis on the genetic dataset from step (a) prior to step (b). In some cases, the regression analysis is a ridge regression or least absolute shrinkage and selection operator (LASSO) regression. In some cases, beta values obtained from the regression analyses performed on the genetic datasets are used to generate the contribution factor in step (b). In some cases, the statistical analysis is a genome-wide association study (GWAS). In some cases, p-values and/or regression weights obtained from the statistical analyses performed on the genetic datasets are used to generate the contribution factors in step (b). In some cases, the obesity phenotype of interest is selected from the group consisting of hungry brain (abnormal satiation), hungry gut (abnormal satiety), slow burn (slow metabolism) or hedonic/emotional eating). In some cases, the obesity phenotype of interest is a hungry brain (abnormal satiation) or hungry gut (abnormal satiety). In some cases, the specific trait in step (b) is total kcal consumed to satiation (CTS) at an ad libitum meal. In some cases, following step (c), the subject is CTS positive if the subject has a ML-GRS for CTS above the sex-specific seventy-fifth percentile or the 4′ quartile, while the subject is CTS negative if the subject has a ML-GRS for CTS below the sex-specific seventy-fifth percentile. In some cases, the plurality of genes in step (a) are two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, TNFRSF11A or DYRK1B. In some cases, the obesity phenotype of interest is a hungry gut (abnormal satiety). In some cases, the specific trait is increased or accelerated baseline gastric emptying for the subject as compared to a control subject, wherein the control subject is not obese. In some cases, the set of genes in step (a) consists of genes known to play role in conferring the hungry gut phenotype. In some cases, the obesity phenotype of interest is a slow burn. In some cases, the specific trait is decreased or low resting energy expenditure (REE) for the subject as compared to a control, wherein the control subject is not obese. In some cases, the set of genes in step (a) consists of genes known to play role in conferring the slow burn phenotype. In some cases, the obesity phenotype of interest is an emotional eating phenotype. In some cases, the specific trait is a finding or result from a behavioral questionnaire indicative of anxiety or emotional eating for the subject. In some cases, the questionnaire is the Hospital Anxiety and Depression Scale (HADS) questionnaire. In some cases, the plurality of genes in step (a) consists of genes known to play role in conferring the emotional eating phenotype.

In another aspect, provided herein is a computer-implemented method for predicting or diagnosing a subject suffering from or suspected of suffering from obesity as possessing abnormal satiation, the method comprising: (a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein the set of genes comprises GLP1R, UCP2, FTO, TCF7L2, MBOAT4 (GOAT), ADRA2A, GNB3. GPBAR1. LEP, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNN13K, LINGO1, FANCL, CADM2, PTBP2, NUDT3, OLFM4, HOXB5, TNFRSF11A and DYRK1B; (b) generating by the computer system, a contribution factor for each SNP located in or near a gene from the set of genes from the genetic dataset for a subject from the population of subjects, wherein the contribution factor is a numerical value, vector, matrix, or function of various biological factors that represents the contribution or effect that each SNP has on total kcal consumed to satiation (CTS) at an ad libitum meal; (c) calculating by the computer system, a gene risk score (GRS) for each gene in the set of genes by integrating the contribution factors generated in step (b) for each SNP for each gene from the set of genes and SNP genotype information located in, around or near each gene in the set of genes, wherein the GRS for each gene represents the cumulative effect of the variation in SNPs located in, around, or near the gene in that particular subject has on the function or regulation of the gene so as to increase or decrease the risk of being above a sex-specific threshold total kcal consumed to satiation (CTS) at an ad libitum meal; (d) iteratively performing by the computer system, steps (a)-(c) for each additional subject from the population of subjects to generate a set of GRSs for each gene in the set of genes, wherein each gene has a GRS for each sex; and(e) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts the CTS by combining gene risk scores and other biological, psychological, or environmental measurements of the subject; (f) training by the computer system, the ML-GRS from step (e) using a classifier model to predict subjects from a training set of samples from a training population of obese subjects as being positive for being above a sex-specific threshold total kcal consumed to satiation (CTS) at an ad libitum meal, thereby generating a trained ML-GRS; and (g) determining by the computer system, if a test subject is positive for the specific trait known to be associated with the obesity phenotype of interest by: (i) calculating by the computer system the ML-GRS for the test subject using steps (a)-(e) on a sample obtained from the test subject; and (ii) comparing by the computer system, the ML-GRS for the test subject to the trained ML-GRS, wherein the test subject is positive for CTS if the ML-GRS for the subject is above the sex-specific 75^{th} percentile for CTS or negative for CTS if the ML-GRS for the subject is below the sex-specific 75^{th} percentile for CTS. In some cases, the sample is selected from the group consisting of a blood sample, a saliva sample, a urine sample, a breath sample, and a stool sample.

In another aspect, provided herein is a method for treating obesity in a subject in need thereof, the method comprising: (a) determining if the subject possesses abnormal satiation using the method of embodiment 46 on a genetic dataset obtained from a sample obtained the subject to determine a ML-GRS for abnormal satiation; and (b) administering a pharmacotherapy to the subject that does not include a GLP-1 agonist if the ML-GRS determined for the subject indicates that the subject is positive for CTS or administering a pharmacotherapy that may include a GLP-1 agonist to the subject if the ML-GRS determined for the subject indicates that the subject is negative for CTS.

In another aspect, provided herein is a method for treating obesity in a subject in need thereof, the method comprising: (a) determining the presence, absence or level of a plurality of gastrointestinal (GI) peptides, a plurality of metabolites, and/or a plurality of genetic variants in a sample obtained from a subject suffering from obesity as well as satiety, satiation, resting energy expenditure and results on a behavioral questionnaire for the subject, thereby generating an obesity analyte signature for the sample; (b) populating a predictive machine learning model with the obesity analyte signature for the subject; and (c) utilizing the predictive machine learning model to predict an obesity phenotype of the subject suffering from obesity, wherein the obesity phenotype is selected from the group consisting of hungry brain (abnormal satiation), hungry gut (abnormal satiety), slow burn (slow metabolism) or hedonic/emotional eating); and (d) administering a pharmacotherapy to the subject that does not include a GLP-1 agonist if the predictive machine learning model to predicts that the subject possesses an abnormal satiation phenotype or administering a pharmacotherapy that may include a GLP-1 agonist to the subject if the predictive machine learning model to predicts that the subject does not possess an abnormal satiation phenotype.

In some cases, the pharmacotherapy that does not include a GLP-1 agonist is phentermine/topiramate. In some cases, the pharmacotherapy that does include a GLP-1 agonist is selected from the group consisting of exenatide, liraglutide, albiglutide, dulaglutide, lixisenatide, semaglutide and tirzepatide.

In yet another aspect, provided herein is a method of treating obesity in a subject in need thereof, the method comprising: (a) detecting an obesity analyte signature in a sample obtained from a subject; wherein the obesity analyte signature is indicative of an obesity phenotype of the subject; and (b) administering a pharmacotherapy to the subject that does not include a GLP-1 agonist if the obesity analyte signature detected in the sample obtained from the subject indicates that the subject possesses an abnormal satiation phenotype or administering a pharmacotherapy that may include a GLP-1 agonist to the subject if the obesity analyte signature detected in the sample obtained from the subject indicates that the subject does not possess an abnormal satiation phenotype. In some cases, the GLP-1 agonist is selected from the group consisting of exenatide, liraglutide, albiglutide, dulaglutide, lixisenatide, semaglutide and tirzepatide.

In another aspect, provided herein is a method of identifying a subject suffering from obesity as a non-responder to treatment with a GLP-1 agonist, the method comprising detecting an obesity phenotype of the subject; and identifying the subject as a non-responder to GLP-1 agonist treatment if the subject is determined to possess an abnormal satiation obesity phenotype.

In some cases, the sample is selected from the group consisting of a blood sample, a saliva sample, a urine sample, a breath sample, and a stool sample. In some cases, the obesity analyte signature comprises the presence of serotonin, glutamine, isocaproic acid, allo-isoleucine, hydroxyproline, beta-aminoisobutyric-acid, alanine, hexanoic acid, tyrosine, and PYY, an absence of 1-methylhistine, gamma-amino-n-butyric-acid, phenylalanine, ghrelin, and a HADS questionnaire result that does not indicate an anxiety subscale (HADS-A; e.g., includes a HADS-A questionnaire result). In some cases, the subject consumes above the seventy-fifth percentile amount of calories at an ad libitum meal. In some cases, the obesity analyte signature comprises the presence of 1-methylhistine, allo-isoleucine, hydroxyproline, beta-aminoisobutyric-acid, alanine, and phenylalanine, an absence of serotonin, glutamine, gamma-amino-n-butyric-acid, isocaproic acid, hexanoic acid, tyrosine, ghrelin, PYY, and does not include a HADS questionnaire result that indicates an anxiety subscale. In some cases, the subject has increased or accelerated baseline gastric emptying as compared to a control subject, wherein the control subject is not obese. In some cases, the obesity analyte signature comprises the presence of 1-methylhistine, serotonin, glutamine, gamma-amino-n-butyric-acid, isocaproic acid, allo-isoleucine, alanine, tyrosine, ghrelin, PYY, an absence of hydroxyproline, beta-aminoisobutyric-acid, hexanoic acid, and phenylalanine, and does includes a HADS questionnaire result that indicates an anxiety subscale. In some cases, the subject has decreased or low resting energy expenditure (REE) for the subject as compared to a control, wherein the control subject is not obese. In some cases, the obesity analyte signature comprises the presence of serotonin, an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and does includes a HADS questionnaire result that indicates an anxiety subscale.

### BRIEF DESCRIPTION OF THE FIGURES

FIG. 1 depicts the study protocol for in-person physiologic testing. After an 8-hour fast, all participants in the study provided a plasma blood sample, had physiologic in-person testing to measure food intake, which included a standardized 320 kcal breakfast, gastric emptying for solids for 4 hours, and an ad libitum meal.

FIGS. 2A-2H depicts the inter-individual variability postprandial satiety and gender differences. FIG. 2A illustrates gastric emptying curve differences between males and females. Half time for gastric emptying for females was 1 hour and 51 min (right vertical dotted line) and for males was 1 hour and 25 minutes (left vertical dotted line). FIG. 2B illustrates visual analogue score (VAS) for hunger in males and females before and after breakfast. FIG. 2C illustrates VAS-fullness in males and females before and after breakfast. FIG. 2D illustrates VAS-satisfaction in males and females before and after breakfast. FIG. 2E illustrates calories consumed by macronutrient composition between females and males. FIG. 2F illustrates total calories to fullness in ad libitum meal in all participants. FIG. 2G illustrates frequency histograms of the density of calories to fullness in males. FIG. 2H illustrates frequency histograms of the density of calories to fullness in females.

FIG. 3 illustrates determinants postprandial satiety variability. Trait variations for each input variable are derived from separate (non-hierarchical) regression models. Values represent the adjusted proportion of variance explained (R2), and error bars show the 95% CI. R2 values were derived using multivariable linear regression.

FIGS. 4A-4H shows the characterization of abnormal satiation. FIG. 4A shows half gastric emptying time of participants with normal and abnormal satiation. FIGS. 4B-4D shows plasma hormone levels (FIG. 4B-Ghrelin; FIG. 4C-PYY; and FIG. 4D-GLP-1) at baseline and 15, 45 and 90 minutes after breakfast (320 kcal). FIGS. 4E-4H shows appetite sensations including satisfaction (FIG. 4E), hunger (FIG. 4F), fullness (FIG. 4G), and desire to eat (FIG. 4H) measured by visual analogue scales before and after breakfast. Data is presented as mean and standard error of the mean.

FIGS. 5A-5B show ROC curves of the predictive model for abnormal satiation (i.e., the machine learning (ML)-driven full phenotype test (FPT)) in the training cohort (FIG. 5A) and in a validation cohort (FIG. 5B).

FIGS. 6A-6B shows total body weight loss % by prediction group with liraglutide (FIG. 6A) and placebo (FIG. 6B). True is responder to GLP-1.

FIG. 7 depicts a diagram of the polygenic risk score (PRS) assessment of the present invention that integrates expert guidance, machine learning, and non-genetic/multi-omic data in order to determine the obesity phenotype of a subject suffering from obesity.

FIGS. 8A-8B show ROC curves of the ML-driven polygenic risk score (PRS) model for appetite in the training cohort (FIG. 8A) and in a validation cohort (FIG. 8B). FIGS. 8C-8D show ROC curves of the PRS model for GLP-1 responder in the training cohort (FIG. 8C) and validation cohort (FIG. 8D).

FIGS. 9A-9B show total body weight loss % in groups predicted as responder vs. non-responder using the PRS model for appetite for patients treated with liraglutide (3 mg; FIG. 9A) or placebo (FIG. 9B). True is responder to GLP-1.

FIGS. 10A-10D shows an experimental design that includes data collection by deep phenotype testing in 717 participants (FIG. 10A), feature extraction for satiation characterization (FIG. 10B), model training and validation for gene risk score development (FIG. 10C) and model extrapolation for prediction of response to anti-obesity medicine (AOM) interventions (FIG. 10D). FIG. 10A illustrates the rigorous testing day starting at 7 am, including resting energy expenditure assessment, blood sampling, radio-labeled breakfast, gastric emptying scans, body composition by DEXA imaging, ad libitum meal test, and behavioral questionnaires. FIG. 10B shows male and (n=179) and female (n=538) satiation distribution by calories to satiation, highlighting factors like genetics, medical records, metabolites, and hormones that contribute to individual satiation responses. FIG. 10C demonstrates the creation and validation of a polygenic risk score (PRS) and machine-learning optimized gene risk score (GRS) for satiation. FIG. 10D depicts the model's effectiveness in predicting response to obesity interventions using outcomes in a 52-week randomized, placebo-controlled trial of phentermine-topiramate ER and a 16-week randomized, placebo-controlled trial of liraglutide by categorizing patients according to CTS by ad libitum meal or by genotype groups.

FIGS. 11A-11F shows a comprehensive analysis of satiation variability and influencing factors that includes inter-Individual variability in satiation (FIG. 11A), variability of satiation explained by input variables (FIG. 11B), sex-related effects on satiation parameters (FIG. 11C), anthropometric influence on satiation (FIG. 11D), body composition influence on satiation (FIG. 11E) and questionnaire-based satiation patterns (FIG. 11F). FIG. 11A illustrates the distribution of satiation responses among 717 participants, highlighting the wide range of caloric intake to satiation. FIG. 11B depicts the proportional variance explained (R2) for satiation variability attributed to individual input variables, obtained from separate non-hierarchical regression models. Error bars represent the 95% confidence interval. R2 values were calculated through multivariable linear regression. FIG. 11C is a violin plot that showcases the distinct impact of sex on satiation parameters, revealing higher caloric intake required to reach fullness in males (p<0.001). FIG. 11D shows a univariate linear regression analyses display the weak correlation between calories to satiation and height in 717 participants. FIG. 11E shows univariate linear regression analyses display the correlation between calories to satiation and total fat percent in 395 participants that completed a DEXA scan. FIG. 11F demonstrates the limited contribution of behavioral questionnaires to explain the variability of objective measurements of satiation as demonstrated by the weak correlation between calories to satiation and scores in the components of the three-factor eating questionnaire: uncontrolled eating, cognitive restrain, and emotional eating completed in 215 participants.

FIG. 12A shows comparative analysis of high and low satiation groups by sex and illustrates the comprehensive distinction between high and low calories to satiation, further categorized as high calories to satiation (n=179) with >977 kcal for females (n=134) and >1374 kcal for males (n=45), and low calories to satiation (n=161) with <650 kcal for females (n=134) and <927 kcal for males (n=44). FIG. 12B shows gastric emptying time by Gender in High and Low Calories to Satiation with significant differences in solid gastric emptying half-time in females (p<0.00l) but not in males (p=0.15). FIG. 12C shows fullness by visual analog scale (VAS) after consuming a 320-kcal breakfast—High Calories to Satiation (n=70) vs. Low Calories to Satiation (n=79). FIG. 12D shows hunger levels after a 320-kcal Breakfast—High Calories to Satiation (n=70) vs. Low Calories to Satiation (n=79). FIG. 12E shows profile of Gastrointestinal Hormones Fasting and Postprandial in High and Low Calories to Satiation: Profile of Peptide YY (PYY) Levels in High (n=83) and Low Satiation (n=59) showing not significant difference between groups at any time point. FIG. 12F shows profile of GLP-1 Levels in High (n=123) and Low Calories to Satiation (n=101) showing only significant differences between groups at 90 minutes. FIG. 12G shows profile of Ghrelin Levels in High (n=85) and Low Calories to Satiation (n=58) showing not significant difference between groups at any time point. FIG. 12H shows the distribution of eating behavior scores in the three-factor eating questionnaire (n=215) (cognitive restraint, uncontrolled eating, emotional eating) between High and Low Calories to Satiation.

FIG. 13A shows a Genome-Wide Association Study (GWAS) for Calories to Satiation. This Manhattan plot displays the results of a GWAS investigating genetic variants associated with calories to satiation in the ad libitum meal. The absence of significant peaks across the genome highlights that no genetic variants reached the threshold for genome-wide significance (p<5×10\hat{ }−8).

FIG. 13B shows a volcano plot of association with calories to satiation traits. Each point represents the association of a variability of a SNP with CTS among male (blue) and female (red) subjects. The x-axis shows the magnitude and direction of the association and the y-axis the significance of the association.

FIG. 13C shows a beeswarm plot of Shapley Additive Explanations (SHAP) values for the CTS_{GRS} model representing the contribution of specific features to model output. Each point for a given feature represents a sample with its horizontal position representing the magnitude and direction that feature effects the probability of high calories to satiation. The color scale indicates the relative value of that feature for that sample as ranked among all other samples. Features are ranked by the mean absolute SHAP value, i.e., magnitude of effect on predictions.

FIGS. 14A-14G illustrates the development and validation of a machine learning assisted gene risk score for high calories to satiation in people with obesity. FIG. 14A shows risk allele distribution for satiation. The number of risk alleles associated with higher calories to satiation was normally distributed in our population. Each circle represents the mean number of calories to satiation during the ad libitum meal test for each risk allele score. The solid line represents the linear regression for the risk allele score and calories to satiation (R2=0.55; β coefficient, 6.78; 95% CI, 5.46 to 8.11). FIG. 14B shows receiver operating characteristics curve for the Machine Learning Assisted Gene Risk Score in the training (n=483), validation (n=50), and independent validation cohort (n=110). FIG. 14C shows caloric intake in males and females according to their Machine Learning Assisted Gene Risk Score (CTS_{GRS}) groups. Participants predicted as low CTS_{GRS} had a lower caloric intake than participants predicted as high CTS_{GRS}. FIGS. 14D-G shows that there were no differences in BMI, or other parameters associated with postprandial satiety when dividing participants according to their CTS_{GRS} group.

FIG. 15 shows a flowchart diagram for the training, validation, and independent testing cohort for development of the machine-learning assisted gene risk score for calories to satiation.

FIG. 16 shows a flowchart diagram for a 12-month, placebo-controlled, randomized clinical trial with phentermine-topiramate extended-release.

FIG. 17 shows a flowchart diagram for a 16-week, placebo-controlled, randomized clinical trial with liraglutide.

FIGS. 18A-18F shows weight loss response by baseline ad libitum buffet meal energy intake and machine-learning assisted gene risk score prediction in response to placebo or phentermine-topiramate ER and placebo vs. liraglutide. FIG. 18A shows weight loss in all patients that participated in the 52-week randomized clinical trial with placebo and phentermine-topiramate ER and underwent satiation testing at baseline. FIG. 18B shows weight loss by sex-stratified satiation by ad libitum buffet meal in patients assigned to placebo and phentermine-topiramate ER, where patients assigned to phentermine and high satiation by ad libitum buffet meal had the greatest total body weight lost percentage. FIG. 18C shows weight loss by sex-stratified satiation by CTS_{GRS} classified as low or high in patients assigned to placebo and phentermine-topiramate ER, where patients assigned to phentermine-topiramate ER and high CTS_{GRS} had the greatest total body weight lost percentage. FIG. 18D shows weight loss in all patients that participated in the 16-week randomized clinical trial of liraglutide vs placebo and underwent satiation testing at baseline. FIG. 18E shows weight loss by sex-stratified satiation by ad libitum buffet meal in patients assigned to liraglutide vs placebo, where patients assigned to liraglutide and low satiation by ad libitum buffet meal had the greatest total body weight lost percentage. FIG. 18F shows weight loss by sex-stratified satiation by CTS_{GRS} in patients assigned to liraglutide vs placebo, where patients assigned to liraglutide and low satiation by CTS_{GRS} had the greatest total body weight lost percentage.

FIGS. 19A-C shows performance of the CTS_{GRS} model in predicting weight loss outcomes of a 12-month, placebo-controlled, randomized clinical trial with phentermine-topiramate extended-release. FIG. 19A shows the CTS_{GRS} model had an AUC of 0.71 for predicting responders achieving at least 15% of total body weight loss at 12 months in participants that were assigned to phentermine-topiramate extended-release. FIG. 19B shows violin plots showing individual weight loss outcomes for participants that completed the 12-month trial stratified by treatment allocation and CTS_{GRS} score. FIG. 19C shows the proportion of responders achieving a total body weight loss superior to 5, 10, and 15% by treatment allocation and CTS_{GRS} group at 12-months.

FIGS. 20A-C shows performance of the CTS_{GRS} model in predicting weight loss outcomes of a 16-week, placebo-controlled, randomized clinical trial with liraglutide. FIG. 20A shows the CTS_{GRS} model had an AUC of 0.75 for predicting responders achieving at least 4% of total body weight loss at 16 weeks in participants assigned to phentermine-topiramate extended-release. FIG. 20B shows violin plots showing individual weight loss outcomes for participants that completed the 16-week trial stratified by treatment allocation and CTS_{GRS} score. FIG. 20C shows the proportion of responders achieving a total body weight loss superior to 4, 5, and 6% by treatment allocation and CTS_{GRS} group at 16-weeks.

FIG. 21A shows total body weight loss percentage (TBWL %) at 3, 6, 9 and 12 months, and FIG. 21B shows individual TBWL % outcomes at 12 Months. Abbreviations: TBWL %: Total body weight loss percentage; HG+: Hungry Gut Positive; HG−: Hungry Gut Negative

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e., can refer to a plural referents. As such, the terms “a” or “an”. “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

The details of one or more embodiments of the invention are set forth in the accompanying drawings and the description below. Other features, objects, and advantages of the invention will be apparent from the description and drawings, and from the claims.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The terms “ad libitum diet” as used herein refer to a diet where the amount of daily calories intake of a subject is not restricted to a particular value. A subject following an ad libitum diet is free to eat till fullness.

The terms “Glucagon-like peptide-1 receptor agonist” or “GLP-1 receptor agonist” as used herein can be used interchangeably with the terms “GLP-1 agonist” or “GLP-1 analog”. Said terms can also be referred to as incretin mimetics. All of the aforementioned terms can refer to agents that act as agonists of the GLP-1 receptor and can work by activating the GLP-1 receptor.

The term “postprandial satiety” as used herein can be interchangeably with “satiety” and refers to the sensation of fullness after a meal termination that perdures through time until hunger returns. Postprandial satiety may overlap with hunger or desire to eat.

As used herein, the term “genotype” can refer to the complete set of genetic material in an organism. Genotype can also be used to refer to the alleles or variants an individual carries in a particular gene or genetic location.

As used herein, the term “allele(s)” can mean any of one or more alternative forms of a gene, all of which alleles relate to at least one trait or characteristic. In a diploid cell, the two alleles of a given gene occupy corresponding loci on a pair of homologous chromosomes. Since the present disclosure, in embodiments, relates to quantitative trait loci (QTLs), i.e., genomic regions that may comprise one or more genes or regulatory sequences, it is in some instances more accurate to refer to “haplotype” (i.e., an allele of a chromosomal segment) instead of “allele”, however, in those instances, the term “allele” should be understood to comprise the term “haplotype”. An “effect allele” as used herein can refer to the less common allele(s) at a particular location in the genome. As used herein, “dosage” can refer to the number of effect alleles in a genotype.

As used herein, the term “nucleotide change” can refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “phenotype” can refer to the observable characteristics of an individual cell, cell culture, organism, or group of organisms, which can result from the interaction between that individual's genetic makeup (i.e., genotype) and the environment.

As used herein, the term “gene” can refer to any segment of DNA associated with a biological function. Thus, genes can include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “SNP” refers to “single nucleotide polymorphisms”, which can refer to substitution of a single nucleotide at a specific position in the genome. The specific location in the genome can possess multiple single-nucleotide alleles. The use of this term in this document should not be construed to exclude the use of any other type of polymorphism in addition to or in place of a SNP such as, for example, sequence insertions, deletions, inversions, and other sequence replacements. Single-nucleotide polymorphisms may fall within coding sequences of genes, non-coding regions of genes, or in the intergenic regions (regions between genes). SNPs within a coding sequence may not necessarily change the amino acid sequence of the protein that is produced, due to degeneracy of the genetic code. SNPs in the coding region can be of two types: synonymous SNPs and nonsynonymous SNPs. Synonymous SNPs do not affect the protein sequence, while nonsynonymous SNPs change the amino acid sequence of protein.

As used herein, the term “homologous” or “homologue” or “orthologue” is known in the art and can refer to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologues” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

The terms “substantially reduced” and “substantially less” are used interchangeably herein and, when referring to an expression level or amount or an activity level of a protein or enzyme, can refer to a lowering of said amount or activity by a percentage or range of percentages as compared to or versus a control or reference level or activity of said protein or enzyme. The terms “substantially reduced” and “substantially less” can refer to a lowering of an amount or level of a protein or enzyme or an activity of an enzyme by at least, at most, exactly or about 1%, 2/a, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% as compared to or versus a control or reference (e.g., a control or reference level or activity of said protein or enzyme). The terms “substantially reduced” and “substantially less” can refer to a lowering of an amount or level of a protein or enzyme or activity of an enzyme (e.g., enzymatic activity) by 1%10-5%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%, 90%, 90%-95% or 95%-100%, inclusive of the endpoints, as compared to or versus a control or reference (e.g., a control or reference level or activity of said protein or enzyme). The terms “substantially reduced” and “substantially less” can also mean that the amount of a protein or enzyme or the activity of an enzyme can be at least, at most, exactly or about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amount of control or reference version of said protein or enzyme or the activity of said enzyme. The terms “substantially reduced” and “substantially less” can also mean that the amount of a protein or enzyme or the activity of an enzyme is 1%-5%, 10%-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95% or 95%400%, inclusive of the endpoints, of the amount of a control or reference version of said protein or enzyme or the activity of an enzyme. With regards to a level or amount of a protein or enzyme, the control or reference can be a level or amount of said protein or enzyme in a control or reference cell. In one embodiment, the tested protein or enzyme in a control of reference cell does not have a heterologous modification. With regards to activity of an enzyme, the control or reference can be the activity of said protein or enzyme in a control or reference cell. In one embodiment, the tested protein or enzyme in a control of reference cell does not have a heterologous modification.

The level or activity of a protein or enzyme provided herein can be measured within a cell or after extraction and/or isolation from a cell (e.g., in vitro). In some cases, the level or amount of a gene encoding a protein of interest is measured or determined. The level or amount of a gene provided herein can be measured within a cell or after extraction from a cell (e.g., in vitro). In some cases, the activity of an enzyme encoded by a gene provided herein is measured or determined. The activity (e.g., specific activity) of an enzyme encoded by a gene provided herein can be measured within a cell or after extraction from a cell (e.g., in vitro). The assay utilized to measure the level or amount of expression of a gene or protein provided herein can be high-throughput in nature. The assay utilized to measure the activity of an enzyme encoded by a gene provided herein can be high-throughput in nature.

The level or amount of a gene provided herein can be measured using any assay known in the art for measuring a level or amount of a gene at the nucleic acid level. Examples of suitable assays for determining or measuring the levels of nucleic acid (e.g., a gene provided herein) can be selected from microarray analysis, RT-PCR such as quantitative RT-PCR (qRT-PCR), serial analysis of gene expression (SAGE), RNA-seq, Northern Blot, digital molecular barcoding technology, for example, Nanostring Counter Analysis, and TaqMan quantitative PCR assays. Other methods of mRNA detection and quantification can be applied, such as mRNA in situ hybridization. mRNA in situ hybridization can be measured using QuantiGene ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples. In one embodiment, the level, presence or amount of gene or genetic variant (e.g., SNP) provided herein can be determined using an array. The array can be a “Genotyping SNP-Chip”.

The level or amount of a protein encoded by a gene provided herein can be measured using any assay known in the art for measuring a level or amount at the protein level. Examples of suitable assays for determining or measuring the levels of protein (e.g., encoded by a gene provided herein) can be selected from quantitative mass spectrometry or immunoassays including, for example, immunohistochemistry, ELISA, Western blot, immunoprecipitation, Luminex® assay, and the like, where a biomarker detection agent such as an antibody, for example, a labeled antibody, specifically binds a protein encoded by a gene provided herein and permits, for example, relative or absolute ascertaining of the amount of a protein in a sample or a cell. The level or amount of an enzyme encoded by a gene provided herein or of the gene itself that has been heterologously modified as provided herein can be compared to the level or amount of the same enzyme or gene that has not been heterologously modified as described herein and the percentage of the level or amount of the modified enzyme or gene vs. the non-modified enzyme or gene can be determined.

The activity of an enzyme encoded by a gene provided herein can be measured using any assay known in the art for measuring enzyme activity. Examples of suitable assays for determining enzyme activity can be any kinase assay known in the art such as, for example, biochemical kinase assays commercially available from EMD Millipore (e.g., FRET-based HTRF assays), eBioscience (e.g., Instant One cell signaling assays), Life Technologies (LanthaScreen or Omnia kinase assays), Symansis (e.g., Multikinase assay array), Abcam or Promega (e.g., the ADP-Glo Kinase Assay).

As used herein, the term “quantitative trait” can refer to a numerical measurement of a physical, chemical, or other aspect, trait or characteristic of an individual.

As used herein, the term “contribution factor” can refer to the apparent amount that the dosage at a particular SNP contributes to a quantitative trait.

As used herein, the terms “polygenic risk score (PRS)”; “polygenic score (PGS)” or “polygenic index (PGI)” can be used simultaneously and can refer to a numerical value indicating the magnitude of risk of a particular phenotype based upon the combined effect of SNPs from multiple locations in the genome. In some cases, the polygenic score (PGS), polygenic risk score (PRS) or polygenic index (PGI) can be a number that summarizes the estimated effect of many genetic variants on an individual's phenotype, typically calculated as a weighted sum of trait-associated alleles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control. In addition, the materials, methods, and examples are illustrative only and not intended to be limiting.

### Overview

Provided herein are methods and systems for predicting whether or not a subject possesses a specific trait. To accomplish this, the methods and systems provided herein rely on data to up-or-down weight a genetic variant, which is located around, within or near a gene known or suspected to play in role in generating the specific trait (e.g., single nucleotide polymorphism (SNP)), contribution to the specific trait, uses systems biology to construct those weights, and uses an open number of gene risk scores (GRSs) for a set of genes known or suspected to play in role in generating the specific trait that are determined by machine learning (ML) feature selection techniques. As such, the methods and system provided herein for determining whether or not a subject possesses a specific trait do not select discrete genetic variant's (e.g., SNPs). As alluded to, the genetic variants can be SNPs, but they could also be some other type of genetic variant such as, for example, an epigenetic feature. The epigenetic feature could be selected from methylation site(s), histone modification(s) or chromatin accessibility site(s). The specific trait could be a trait associated with an obesity phenotype as described herein. The specific trait associated with an obesity phenotype as provided herein could be selected from the group consisting of total kcal consumed to satiation (CTS) at an ad libitum meal, gastric emptying rate, resting energy expenditure (REE), results from a behavioral questionnaire indicative of anxiety or emotional eating and any combination thereof. The specific trait could also be, for example, weight loss or comorbidity change in response to weight loss, or response to an obesity intervention. The obesity intervention could be an anti-obesity medication (AOM), or a behavioral and/or lifestyle intervention. The AOM could be phentermine-topiramate or a GLP-1 agonist. The data used to up or down weight a genetic variant can be genetic and/or non-genetic data. The genetic data can include, but is not limited to, cell-type-specific epigenetic activity, gene regulatory activity, or chromatin structure and/or genomic positioning. In any method or system provided herein, a GRS is a multidimensional integration function with dimensions of genotype, trait contribution, genomic contribution as input. In any method or system provided herein, the method or system can further comprise iteratively removing genes from the set of genes whose gene risk scores are deemed not informative for the specific trait and subsequently combine the remaining GRSs with other non-genetic and/or multi-omic data for a subject with machine learning models to generate a machine-learning assisted GRS for the specific trait. The ML-GRS can then be used to predict or diagnose a de novo subject as possessing the specific trait for which the ML-GRS was constructed. The non-genetic and/or multi-omic data can be selected from the group consisting of metabolomic data, proteomic data, peptidomic data, epigenetic data, microbiome data, results from one or more questionnaires and any combination thereof. The epigenetic data can be the presence or absence of DNA modifications such as methylation, histone modifications, chromatin accessibility, etc.

In one aspect, provided herein are methods and systems for predicting whether or not a subject possesses a specific trait associated with an obesity phenotype of an individual or subject with obesity from one or more samples obtained from said individual or subject with obesity. In one embodiment, the methods and systems utilize machine learning in order to predict the obesity phenotype of the subject with obesity. In one embodiment, the methods and system utilizes contribution factors and gene risk scores (GRS) to determine the obesity phenotype of the subject with obesity. The gene risk scores can integrate expert guidance, machine learning, and non-genetic/multi-omic data. In one embodiment, the obesity phenotype of an individual with obesity can be ascertained using an adaptive, contribution factor based GRS as provided herein. The adaptive, contribution factor based GRS can be tailored to and/or used for determining or diagnosing a specific obesity phenotype such as, for example, hungry brain, hungry gut, slow burn or hedonic eating as described in US20210072259A1, which is herein incorporated by reference in its entirety. The adaptive, contribution factor based GRS can utilize expert guidance, machine learning and non-genetic/multi-omic data. The non-genetic/multi-omic data can comprise data obtained from determining the metabolome, the proteome, and/or the peptidome of an individual (e.g., an individual with obesity) of a sample obtained from the obese subject. In some cases, the non-genetic/multi-omic data can be identified in a sample obtained from individual with obesity.

In one embodiment, the methods and systems for ascertaining an obesity phenotype of an individual or subject utilizes a regression, machine learning driven model for appetite. The regression machine learning model can be a multi-variate logistic regression, machine learning driven model. In one embodiment, the multi-variate logistic regression, machine learning driven model can be for appetite and can utilize a myriad of genetic and non-genetic multi-omic data. The myriad of genetic and non-genetic multi-omic data used in a multi-variate logistic regression machine learning-driven model for appetite provided herein can be referred to as the full phenotype tests or FPTs.

Also provided herein are methods and systems for predicting or stratifying individuals with obesity into sub-populations of responders and non-responders for one or more interventions. In one embodiment, a machine learning based system or method provided herein for use in determining the obesity phenotype of a subject or individual with obesity can be used to select said subject or individual with obesity for treatment with one or more interventions for treating obesity. In this manner, the machine learning based system or method provided herein for use in determining the obesity phenotype of a subject or individual with obesity can serve as a companion diagnostic to an intervention for treating obesity. The one or more interventions can be selected from the group consisting of a pharmacological intervention, a surgical intervention, a weight loss device, a diet intervention, a lifestyle intervention, a behavior intervention, and a microbiome intervention).

In one embodiment, provided herein is a method of treating obesity in a subject in need thereof, the method comprising: (a) determining whether or not the subject possesses an abnormal satiation or hungry brain phenotype; and (b) administering a pharmacotherapy to the subject that does not include a GLP-1 agonist if the obesity analyte signature detected in the sample obtained from the subject indicates that the subject possesses an abnormal satiation phenotype or administering a pharmacotherapy that may include a GLP-1 agonist to the subject if the obesity analyte signature detected in the sample obtained from the subject indicates that the subject does not possess an abnormal satiation phenotype. In some cases, step (b) comprises administering phentermine-topiramate pharmacotherapy if the obesity analyte signature detected in the sample obtained from the subject indicates that the subject possesses an abnormal satiation phenotype. In some cases, step (a) is performed by detecting an obesity analyte signature in a sample obtained from a subject; wherein the obesity analyte signature is indicative of an obesity phenotype of the subject. In some cases, step (a) is performed using a ML-GRS developed for predicting the abnormal satiation or hungry brain phenotype as provided herein. In some cases, step (a) is performed using a machine learning, full phenotype test for abnormal satiation or hungry brain phenotype as provided herein. The GLP-1 agonist can be any agent known in the art whose mechanism of action comprises activating the GLP-1 receptor. GLP-1 agonists for use in a method and/or system provided herein can include, but are not limited to, exenatide, liraglutide, albiglutide, dulaglutide, lixisenatide, semaglutide, tirzepatide or any combination thereof.

In one embodiment, provided herein is a method of identifying a subject suffering from obesity as a non-responder to treatment with a GLP-1 agonist, the method comprising detecting an obesity phenotype of the subject, and identifying the subject as a non-responder to GLP-1 agonist treatment if the subject is determined to possess an abnormal satiation obesity phenotype. In some cases, a non-responder to GLP-1 agonist treatment may be a responder to phentermine-topiramate pharmacotherapy if the subject is determined to possess an abnormal satiation obesity phenotype. In one embodiment, the detection of the obesity phenotype of the subject can comprise utilizing a machine learning based system or method provided herein for use in determining the obesity phenotype of a subject or individual with obesity. In one embodiment, the detection of the obesity phenotype of the subject is performed using a ML-GRS developed for predicting a specific trait associated with the abnormal satiation or hungry brain phenotype as provided herein. The specific trait can be total kcal consumed to satiation (CTS) at an ad libitum meal. In one embodiment, the detection of the obesity phenotype of the subject is performed using by determining using a machine learning, full phenotype test for abnormal satiation or hungry brain phenotype as provided herein. The GLP-1 agonist can be any agent known in the art whose mechanism of action comprises activating the GLP-1 receptor. GLP-1 agonists for use in a method and/or system provided herein can include, but are not limited to, exenatide, liraglutide, albiglutide, dulaglutide, lixisenatide, semaglutide, tirzepatide or any combination thereof.

A subject or individual can be a mammal. Any type of mammal can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. In some cases, the mammal can be a human. In some cases, a mammal can be a mammal with obesity. For example, humans with obesity can be assessed for intervention (e.g., lifestyle intervention and/or a pharmacological intervention) responsiveness and treated with one or more interventions as described herein. In cases where mammal is a human, the human can be of any race. For example, a human can be Caucasian or Asian.

Any appropriate method can be used to identify a mammal or individual as being overweight (e.g., as having obesity). In some cases, calculating body mass index (BMI), measuring waist and/or hip circumference, health history (e.g., weight history, weight-loss efforts, exercise habits, eating patterns, other medical conditions, medications, stress levels, and/or family health history), physical examination (e.g., measuring your height and examining your abdomen), percentage of body fat and distribution, percentage of visceral and organs fat, metabolic syndrome, and/or obesity related comorbidities can be used to identify mammals (e.g., humans) as having obesity. For example, a BMI of greater than about 30 kg/m² can be used to identify mammals (e.g., Caucasian humans) as having obesity with or without a co-morbidity such as type 2 diabetes. For example, a BMI of grater than about 27 kg/m² with a co-morbidity can be used to identify mammals (e.g., Asian humans) as having obesity.

The individual can also have one or more obesity-related (e.g., weight-related) co-morbidities. Examples of weight-related co-morbidities include, without limitation, hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis, depression, anxiety, and atherosclerosis (coronary artery disease and/or cerebrovascular disease). In some cases, the methods and materials described herein can be used to treat one or more obesity-related co-morbidities.

A sample can be any type of sample that can be obtained from a subject. In some cases, a sample can be a biological sample. In some cases, a sample can contain obesity analytes (e.g., DNA, RNA, proteins, peptides, metabolites, hormones, and/or exogenous compounds (e.g., medications)). Examples of samples that can be assessed as described herein include, without limitation, fluid samples (e.g., blood, serum, plasma, urine, saliva, sweat, or tears), breath samples, cellular samples (e.g., buccal samples), tissue samples (e.g., adipose samples), stool samples, gastrointestinal mucosa samples. In some cases, a sample (e.g., a blood sample) can be collected while the mammal is fasting (e.g., a fasting sample such as a fasting blood sample). In some cases, a sample can be processed (e.g., to extract and/or isolate obesity analytes).

### Gene Risk Score Model

In one embodiment, the obesity phenotype of an individual with obesity can be ascertained using an adaptive, contribution factor based gene risk score (GRS) as provided herein. A GRS as provided herein can utilize a basic regression model or a statistical model as known in the art. In some cases, an adaptive, contribution factor based GRS can be generated for a specific obesity phenotype. The specific obesity phenotype can be any obesity phenotype known in the art, such as, for example hungry brain, hungry gut, slow burn or hedonic/emotional eating as described in US20210072259A1, which is herein incorporated by reference in its entirety. In some cases, provided herein is a suite of adaptive, contribution factor based GRSs that can integrate data obtained from a sample obtained from a subject with obesity to ascertain said subject's obesity phenotype. The suite of adaptive, contribution factor based GRSs can comprise adaptive, contribution factor based GRSs developed for each obesity phenotype (e.g., hungry brain, hungry gut, slow burn or hedonic eating). The suite of adaptive, contribution factor based GRSs can be part of a system provided herein for determining a subject's obesity phenotype. Any obesity phenotype specific, adaptive, contribution factor based GRS can utilize expert guidance, machine learning and non-genetic/multi-omic data. The non-genetic/multi-omic data can comprise measuring or assessing the metabolome, the proteome, and/or the peptidome of an individual (e.g., an individual with obesity). In some cases, the non-genetic/multi-omic data can be identified in a sample obtained from an individual with obesity. The multi-omic data obtained from a sample obtained from an individual with obesity can represent an obesity analyte signature for that individual.

In one aspect, provided herein is a method of predicting or diagnosing an obesity phenotype of subject suffering from or suspected of suffering from obesity that utilizes a machine learning assisted gene risk score (ML-GRS). In some cases, the method of predicting or diagnosing an obesity phenotype of a subject suffering from or suspected of suffering from obesity that utilizes a ML-GRS is a computer implemented method. In some cases, the method of predicting or diagnosing an obesity phenotype of a subject suffering from or suspected of suffering from obesity that utilizes a ML-GRS is an in silico method. The GRS for use in this method can be an adaptive, contribution factor based GRS that can utilize expert guidance for selecting specific genes of interest for calculating gene risk scores, machine learning and non-genetic/multi-omic data as provided herein (e.g., Example 4). A subject can be determined to be suffering from obesity if they are determined to possess a BMI of greater than about 30 kg/m2 without a co-morbidity or a BMI of greater than about 27 kg/m2 with a co-morbidity. Generation of a ML-GRS for use in predicting an obesity phenotype of interest (e.g., hungry brain (abnormal satiation), hungry gut (abnormal satiety), slow burn (slow metabolism) or hedonic/emotional eating) in a subject suffering from obesity can comprise: (a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, and wherein each gene in the set of genes is known or suspected to play a role in obesity; (b) generating by the computer system, a contribution factor for each SNP located in, around or near a gene from the set of genes from the genetic dataset for a subject from the population of subjects; (c) calculating by the computer system, a gene risk score (GRS) for each gene in the set of genes by integrating the contribution factors generated in step (b) for each SNP for each gene from the set of genes and SNP genotype information located in, around, or near each gene in the set of genes, wherein the GRS for each gene represents the cumulative effect of the variation in SNPs located in, around, or near the gene in that particular subject has on the function or regulation of the gene so as to increase or decrease the risk of the subject possessing a specific trait known to be associated with the obesity phenotype of interest; (d) iteratively performing by the computer system, steps (a)-(c) for each additional subject from the population of subjects to generate a set of GRSs for each gene in the set of genes from the population of subjects, wherein each gene has a GRS for each sex; (e) integrating by the computer system, the GRSs from step (d) with non-genetic and/or multi-omic data, which can be any such data provided herein and/or known in the art, obtained from the samples obtained from each subject from the population of subjects using a machine learning model to calculate a machine learning assisted gene risk score (ML-GRS) for the specific trait known to be associated with the genotype of interest; (f) training by the computer system, the ML-GRS from step (e) using a classifier model to predict subjects from a training set of samples from a training population of obese subjects as being positive for the specific trait known to be associated with the obesity phenotype of interest, thereby generating a trained ML-GRS. In some cases, the method further comprises: (g) determining by the computer system, if a test subject is positive for the specific trait known to be associated with the obesity phenotype of interest by: (i) calculating by the computer system the ML-GRS for the test subject using steps (a)-(c) on a sample obtained from the test subject; and (ii) comparing by the computer system, the ML-GRS for the test subject to the trained ML-GRS. The test subject can be determined to be negative for the specific trait known to be associated with the obesity phenotype of interest if the ML-GRS for the test subject is below a sex-specific percentile. In some cases, the sex-specific percentile is at least the 60^{th}, 65^{th}, 70^{th}, 75^{th}, 80^{th}, 85^{th}, 90^{th}, 95^{th} or 99^{th} percentile for the specific trait known to be associated with the obesity phenotype of interest. In some cases, the sex-specific percentile is between the 60^{th}-65^{th}, 65^{th}-70^{th}, 70^{th}-75^{th}, 75^{th}-80^{th}, 80^{th}-85^{th}, 85^{th}-90^{th}, 90^{th}-95^{th} or 95^{th}-99^{th} percentile for the specific trait known to be associated with the obesity phenotype of interest. The test subject can be determined to be negative for the specific trait known to be associated with the obesity phenotype of interest if the ML-GRS for the test subject is below a sex-specific percentile. In some cases, the sex-specific percentile is at least the 10^{th}, 15^{th}, 20^{th}, 25^{th}, 30^{th}, 35^{th}, 40^{th}, 45^{th}, 50^{th}, 55^{th} or 60^{th} percentile for the specific trait known to be associated with the obesity phenotype of interest. In some cases, the sex-specific percentile is between the 10^{th}-15^{th}, 15^{th}-20^{th}, 20^{th}-25^{th}, 25^{th}-30^{th}, 30^{th}-35^{th}, 35^{th}-40^{th}, 40^{th}-45^{th} or 45^{th}-50^{th} percentile for the specific trait known to be associated with the obesity phenotype of interest. In some cases, the test subject can be determined to be positive for the specific trait known to be associated with the obesity phenotype of interest if the ML-GRS for the test subject is above the sex-specific 75^{th} percentile for the specific trait known to be associated with the obesity phenotype of interest or negative for the specific trait known to be associated with the obesity phenotype of interest if the ML-GRS for the subject is below the sex-specific 75^{th} percentile for the specific trait known to be associated with the obesity phenotype of interest. In this way, the final ML-GRS model can provide a binary decision on whether or not the test subject possesses the specific trait or not. In some cases, the SNP genotype information can comprise risk allele dosage, major allele presence, occurrence of a de novo variant, insertion, deletion, or genome rearrangement. In some cases, each gene in the set of genes is known or suspected to play a role in appetite regulation, energy expenditure, lipid metabolism or adipogenesis.

In some cases, the classifier model in step (e) can be selected from the group consisting of a support vector classifier (SVC), gradient boosted machines (GBM), Neural networks (NN), convolutional neural networks (CNN), Logistic regression (e.g., LASSO and Ridge regression), classification and regression trees (CRT), Bayesian inference, and clustering methods.

The contribution factor can be a numerical value, vector, matrix, or function of various biological factors that represents the contribution or effect that each SNP has on a specific trait known to be associated with the obesity phenotype of interest. The various biological factors can be non-genetic and/or multi-omic data. The non-genetic and/or multi-omic data can be selected from the group consisting of metabolomic data, proteomic data, peptidomic data, epigenetic data, microbiome data, results from one or more questionnaires and any combination thereof. The epigenetic data can comprise a presence or absence of DNA modifications selected from the group consisting of methylation modifications, histone modifications and chromatin accessibility. The microbiome data can be from any site on a subject or a subject's environment. For example, but in no way limiting, the microbiome data can be from a stool sample, a skin sample, or a sample from the subject's environment.

In some cases, the contribution factor is a function or algorithm that utilizes output of a regression analysis or a statistical analysis on the genetic dataset as well as one or more additional co-factors, covering regression against one or more traits using one or more types of genetic datasets selected from the group consisting of a genome-wide data microarray chip (GWAS), targeted sequencing (exome or targeted genetic panel), variants detected by qPCR through targeted amplification, whole genome sequencing (WGS), targeted or untargeted sequencing of genomic re-arrangement, deletions, duplications, repeat extensions, and complex genotyping of HLA and CYP genes. In some cases, the results from multiple regressions can be combined into a single contribution factor. In some cases, the one or more additional co-factors relate to biological factors regarding genetics, epigenetics, and gene regulation for each SNP. In some cases, the one or more additional co-factors are selected from the group consisting of proximity of the SNP to the specific gene, known or theoretical mechanistic role of the SNP metadata regarding the SNP in relationship to the gene, and any combination thereof. The known or theoretical mechanistic role of the SNP can be selected from the group consisting of synonymous mutation, nonsynonymous mutation, frameshift mutation and nonsense mutation. The known or theoretical mechanistic role of the SNP can affect the three-dimensional structure of the translated protein binding sites, protein-protein interaction sites and modification sites. The modification sites can be selected from the group consisting of phosphorylation, glycosylation and proteolytic site. In some cases, the mechanistic role comprises whether or not the SNP is within an intron, exon, enhancer region or regulatory region of the gene, a cis-regulatory element, a promoter region, a non-coding exonic region, a coding exonic region, intronic region, splice site, transcription factor binding site, epigenetic modification site, at a remote genomic location involved in a three-dimensional chromatin contact with the gene, or a cell-type-specific topological-associated domain that contains the gene. The cis-regulatory element can be an enhancer or insulator. In some cases, the epigenetic modification site comprises methylation modifications, histone modifications, and/or chromatin accessibility.

In some cases, any method or system provided herein for generating a ML-GRS step (e) comprises a forward feature selection, backward feature selection, or random feature sampling algorithm and a random forest, GBM, or linear predictor that iteratively selects a feature with a desired training accuracy using the random forest predictor in multiple rounds until no further training accuracy improvement is detected.

In some cases, each GRS generated in a method or system provided herein for generating a ML-GRS provides an indication of a role or effect that the SNPs located around or near a specific gene may play on the specific trait known to be associated with the obesity phenotype of interest.

In some cases, any method or system provided herein for generating a ML-GRS further comprises performing a genetic analysis on a sample obtained from the subject suffering from or suspected of suffering from obesity prior to step (a). In some cases, the genetic analysis comprises obtaining sequence reads from the whole or portions of the whole genome of the subject. In some cases, the genetic analysis comprises performing a genotyping method selected from the group consisting of restriction fragment length polymorphism identification (RFLPI), random amplified polymorphic detection (RAPD), amplified fragment length polymorphism detection (AFLPD), polymerase chain reaction (PCR), DNA sequencing. RNA sequencing, allele specific oligonucleotide (ASO) probes, and hybridization to microarrays or beads.

In some cases, any method or system provided herein for generating a ML-GRS further comprises performing by the computer system, a regression analysis and/or statistical analysis on the genetic dataset from step (a) prior to step (b). In some cases, the regression analysis is a ridge regression or least absolute shrinkage and selection operator (LASSO) regression. In some cases, the beta values obtained from the regression analyses performed on the genetic datasets are used to generate the contribution factor in step (b). In some cases, the statistical analysis is a genome-wide association study (GWAS). In some cases, p-values and/or regression weights obtained from the statistical analyses performed on the genetic datasets are used to generate the contribution factors in step (b). In some cases, performing the regression analysis or statistical analysis on the genetic dataset occurs following the genetic analysis on the sample as described herein such that the genetic dataset obtained from the genetic analysis (e.g., genotyping method) is used for the regression or statistical analysis. In some cases, the method for generating the ML-GRS further comprises performing a regression analysis on the genetic dataset obtained from the sample obtained from the subject prior to generating the contribution factor. The regression analysis can be any regression analysis known in the art and appropriate for the variables (i.e., predictor variable(s) and response variable) utilized in the analysis. In one embodiment, the regression analysis is a ridge regression or least absolute shrinkage and selection operator (LASSO) regression. In one embodiment, beta values obtained from the regression analyses (e.g., LASSO regression) performed on the genetic datasets are used to generate the contribution factors as described herein. In some cases, the method for generating the ML-GRS further comprises performing a statistical analysis on the genetic dataset obtained from the sample obtained from the subject prior to generating the contribution factor. The statistical analysis can be any statistical analysis known in the art and appropriate for determining the correlation between SNPs and a particular obesity phenotype (e.g., hungry brain, hungry gut, emotional eating and slow burn). In one embodiment, the statistical analysis is a genome-wide association study (GWAS). In one embodiment, p-values obtained from the statistical analyses (e.g., GWAS) performed on the genetic datasets are used to generate the contribution factors as described herein.

It should be noted that an alternative for using SNPs in a method for generating an ML-GRS for a specific trait or phenotype of a subject (e.g., a specific trait associated with an obesity phenotype of interest) as provided herein can entail using epigenetic data form the subject or population of subjects. Examples of epigenetic data that can be used include, but is not limited to, epigenetic data on histone modification sites. Histone modifications that can used to generate a ML-GRS as provided herein can be selected from the group consisting of acetylation, methylation, phosphorylation, ubiquitylation, GlcNAcylation, citrullination, crotonylation, sumoylation, isomerization and combinations thereof.

The ML-GRS in any method provided herein can predict if any one subject possesses the specific trait known to be associated with the obesity phenotype of interest by combining gene risk scores and additional biological, psychological, or environmental measurements of the any one subject. The additional biological, psychological or environmental measurements can include any non-genetic and/or multi-omic data provided herein.

In some cases, the gene risk scores can be normalized prior to training the SVC. The normalization can be performed using a Yeo power transform and can entail creating synthetic samples for training using, for example, SMOTE sampling. In one embodiment, the GSRs are normalized with a Yeo power transformation and the height of the individual using a support vector classifier (SVC). The SVC was trained using a linear kernel and a C value of 0.001 using SMOTE oversampling.

In some cases, the ML-GRS for the obesity phenotype of interest predicts the obesity phenotype of interest with a sensitivity and/or specificity is at least 65%. In some cases, the ML-GRS for the obesity phenotype of interest predicts the obesity phenotype of interest with a sensitivity and/or specificity is at least 72%. In some cases, the ML-GRS for the obesity phenotype of interest predicts the obesity phenotype of interest with an area under the curve (AUC) of at least 0.60, 0.65, 0.7, 0.75, 0.8, 0.85, 0.9 0.95 or 1. In some cases, the ML-GRS for the obesity phenotype of interest predicts the obesity phenotype of interest with positive predict value of at least 50%. The prediction can be as compared to a control. In one embodiment, the control is a patient or population of patients previously determined to possess the specific obesity phenotype the ML-GRS is developed to predict using any method known in the art such as those taught in US20210072259A1 and/or WO2022246203A1, both which are herein incorporated by reference in their entireties. The obesity of interest can be selected from the group consisting of hungry brain (abnormal satiation), hungry gut (abnormal satiety), slow burn (slow metabolism) and hedonic/emotional eating.

As noted previously herein, the specific trait on which an ML-GRS can be built as described herein can vary. In some cases, the ML-GRS can be built around response to an obesity treatment. The obesity treatment could be a pharmacological or non-pharmacological treatment. The pharmacological treatment can be response to phen-top treatment or response to GLP-1 agonist treatment.

In one embodiment, the obesity phenotype of interest is the hungry brain or abnormal satiation obesity phenotype. Further to this embodiment, the specific trait in step (b) is consuming above the seventy-fifth percentile amount of calories (e.g., kcal) at an ad libitum meal (CTS). The set of genes in step (a) comprises or consists of the genes listed in Table 5. In some cases, the set of genes can comprise or consist of two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, TNFRSF11A or DYRK1B. In some cases, the set of genes can comprise or consist of two or more genes selected from GLP1R, UCP2, FTO, TCF7L2, MBOAT4 (GOAT), ADRA2A, GNB3, LEPR. SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY5R, NR1H4, UCP3, FGFR4, GHRL, MC4R, POMC, AGRP, GIPR or TFAP2B. In some cases, steps (a)-(d) produce a defined set of GRSs across the set of genes. In one embodiment, steps (a)-(d) produce GRSs across the set of genes that optimally maximize the true positive rate while minimizing the false positive rate for predicting the hungry brain phenotype. In one embodiment, steps (a)-(d) produce GRSs across a plurality of genes that are predictive of the hungry brain phenotype with the desired sensitivity, specificity, AUC and/or PPV as provided herein. In some cases, the plurality of genes for which steps (a)-(d) produce a defined set of GRSs consists essentially of, consists of or comprises SIM1, PCSK1, SH2B1, LEPR, UCP2, FTO, TCF7L2, GLP1R, TNFRSF11A, and ADRA2A or any combination thereof. A ML-GRS generated for determining if a test subject possesses abnormal satiation (i.e., high CTS as described herein such as in Example 4) can be referred to as CTS_{GLR}. In one embodiment, the obesity phenotype of interest is the hungry gut or abnormal satiety obesity phenotype. Further to this embodiment, the specific trait in step (b) is an increased or accelerated baseline gastric emptying for the subject as compared to a control (e.g., individual without obesity. The gastric emptying can be measured using any method known in the art such as, for example, scintigraphy and can be represented as GE T1/4 or GE T1/2. The set of genes in step (a) comprises or consists of genes known to play role in conferring the hungry gut phenotype. The set of genes in step (a) comprises or consists of the genes listed in Table 5. In some cases, the set of genes can comprise or consist of two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL. MC4R, MC3R, POMC, AGRP, GIPR, CNR1, FAAH, GCG, CELA2A, PPARG, TFAP2B, APOE, TNFRSF11A or DYRK1B. In some cases, the set of genes can comprise or consist of two or more genes selected from GLP1R, UCP2, FTO, TCF7L2, MBOAT4 (GOAT), ADRA2A, GNB3, LEPR, SH2B1, SIM1, NCOA1 (SRC1), PCSK1, TMEM18, NEGR1, BDNF, GPRC5B, GNPDA2. MTCH2, KCTD15, SEC16B, FAIM2, TNNI3K, LINGO1, FANCL, CADM2, PTBP2, NUDT3, OLFM4, HOXB5, NPY1R, NPY5R, NR1H4, UCP3, FGFR4, GHRL, MC4R, POMC, AGRP, GIPR or TFAP2B. In some cases, steps (a)-(d) produce a defined set of GRSs across a plurality of genes. In one embodiment, steps (a)-(d) produce GRSs across a plurality of genes that optimally maximize the true positive rate while minimizing the false positive rate for predicting the hungry gut phenotype. In one embodiment, steps (a)-(d) produce GRSs across a plurality of genes that are predictive of the hungry gut phenotype with the desired sensitivity, specificity, AUC and/or PPV as provided herein.

In one embodiment, the obesity phenotype of interest is the slow burn obesity phenotype. Further to this embodiment, the specific trait in step (b) is a decreased or low resting energy expenditure (REE) for the subject as compared to a control (e.g., individual without obesity. The REE can be measured using any method known in the art such as, for example, indirect calorimetry and can be represented as kcal/24 hrs. The set of genes in step (a) comprises or consists of genes known to play role in conferring the slow burn phenotype. The set of genes in step (a) comprises or consists of the genes listed in Table 5. In some cases, the set of genes can comprise or consist of two or more genes selected from GLP1R, UCP2, FTO, TCF7L2, MBOAT4 (GOAT), ADRA2A, GNB3, GPBAR1, LEP, LEPR, SH2B, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, TNFRSF11A or DYRK1B. In some cases, the set of genes can comprise or consist of two or more genes selected from GLP1R, UCP2, FTO. TCF7L2, MBOAT4 (GOAT), ADRA2A, GNB3, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY5R, NR1H4, UCP3, FGFR4, GHRL, MC4R, POMC, AGRP, GIPR or TFAP2B. In some cases, steps (a)-(d) produce a defined set of GRSs across a plurality of genes. In one embodiment, steps (a)-(d) produce GRSs across a plurality of genes that optimally maximize the true positive rate while minimizing the false positive rate for predicting the slow burn phenotype. In one embodiment, steps (a)-(d) produce GRSs across a plurality of genes that are predictive of the slow burn phenotype with the desired sensitivity, specificity. AUC and/or PPV as provided herein.

In one embodiment, the obesity phenotype of interest is the emotional eating obesity phenotype. Further to this embodiment, the specific trait in step (b) is a finding or result from a behavioral questionnaire indicative of anxiety or emotional eating for the subject. The questionnaire can be any questionnaire known in the art for assessing anxiety and/or emotional eating, for example, the Hospital Anxiety and Depression Scale (HADS) questionnaire. The set of genes in step (a) comprises or consists of genes known to play role in conferring the emotional eating phenotype. The set of genes in step (a) comprises or consists of the genes listed in Table 5. In some cases, the plurality of genes can comprise or consist of two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG. TFAP2B, APOE, TNFRSF11A or DYRK1B. In some cases, the set of genes can comprise or consist of two or more genes selected from GLP1R, UCP2, FTO, TCF7L2, MBOAT4 (GOAT), ADRA2A, GNB3, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY5R, NR1H4, UCP3, FGFR4, GHRL, MC4R, POMC, AGRP, GIPR or TFAP2B. In some cases, steps (a)-(d) produce a defined set of GRSs across a plurality of genes. In one embodiment, steps (a)-(d) produce GRSs across a plurality of genes that optimally maximize the true positive rate while minimizing the false positive rate for predicting the emotional eating phenotype. In one embodiment, steps (a)-(d) produce GRSs across a plurality of genes that are predictive of the emotional eating phenotype with the desired sensitivity, specificity, AUC and/or PPV as provided herein.

It is to be noted that the phenotype, Hungry Brain, as used herein can be defined based on a threshold applied to separate a range of CTS values into categories above the 75% ile. However, other thresholds and logics can define additional phenotypes. One example is the Slow Burn phenotype, which can be defined as the subjects with a resting energy expenditure <the 25% ile. The threshold and logic can be further altered to express phenotypes, such as <10% ile for an ‘extreme’ phenotype, or between a range, such as 40% ile to 60% ile of CTS being a ‘borderline hungry gut’. A % ile may be optimized to separate distinct phenotypes, which could result in a % ile threshold from 1-99%. The threshold may or may not be sex-specific, but could be further specified, such as having a different CTS threshold for subjects of different genetic ancestries or having the presence or absence of another designation, such as having uncontrolled type II diabetes. The phenotype can also be defined by a categorical designation of a subject in the absence of any ranged values to which a threshold is applied, such as the presence of coronary artery disease or congestive heart failure. Finally, the phenotype can be defined by a combination of one or more ranged values and designations, such as an ‘emotional hunger-slow burn’ using % ile thresholds being applied to both HADS score and resting energy expenditure, or an ‘early onset hungry brain’ wherein a subject has >50% ile CTS and history of obesity prior to age 13. The ML-GRS can be tailored to any of these approaches to specifying a phenotype at every stage of development: gene selection, trait & contribution factor selection, feature selection, and ML model training.

In some cases, the ML-GRS for the obesity phenotype of interest predicts the obesity phenotype of interest with a sensitivity of about, at most, at least 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. In some cases, the ML-GRS for the obesity phenotype of interest predicts the obesity phenotype of interest with a sensitivity of at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between.

In some cases, the ML-GRS for the obesity phenotype of interest predicts the obesity phenotype of interest with a specificity of about, at most, at least 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. In some cases, the ML-GRS for the obesity phenotype of interest predicts the obesity phenotype of interest with a specificity of at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between.

In some cases, the ML-GRS for the obesity phenotype of interest predicts the obesity phenotype of interest with positive predictive value of about, at most, at least 50%, 51%, 52%, 53%, 54%, 55%, 56%, 57%, 58%, 59%, 60%, 61%, 62%, 63%, 64%, 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. In some cases, the ML-GRS for the obesity phenotype of interest predicts the obesity phenotype of interest with a PPV of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between.

A SNP can be considered to be located around or near a specific gene if it is located within at least, at most, about or exactly 1, 10, 50, 100, 150, 200, 250, 300, 350, 400, 450, 500, 600, 700, 800, 900, 1,000, 2,000, 3,000, 4,000, 5,000, 6,000, 7,000, 8,000, 9,000, 10,000, 15,000, 20,000, 30,000, 40,000, 50,000, 60,000, 70,000, 80,000, 90,000, 100,000, 200,000, 300,000, 400,000, 500,000, 600,000, 700,000, 800,000, 900,000 or 1,000,000 kilobases (KBs) of the specific gene. A SNP is considered to be located around or near a specific gene if it is located within about 1 kb to about 100 kb, about 100 kb to about 250 kb, about 250 kb to about 500 kb, about 500 kb to about 1,000 kb, about 1,000 kb to about 2,000 kb, about 2,000 kb to about 5,000 kb, about 5,000 kb to about 10,000 kb, about 10,000 kb to about 20,000 kb, about 20,000 kb to about 50,000 kb, about 50,000 kb to about 100,000 kb, about 100,000 kb to about 500,000 kb or about 500,000 kb to about 1,000,000 kbs of the specific gene.

### Machine Learning, Full Phenotype Test Model

In yet another aspect, provided herein is a method, which can be computer implemented, for determining an obesity phenotype of a subject suffering from obesity, the method comprising: (a) determining the presence, absence or level of a plurality of gastrointestinal (GI) peptides, a plurality of metabolites, and/or a plurality of genetic variants in a sample obtained from a subject suffering from obesity, thereby generating an obesity analyte signature for the sample; (b) determining satiety, satiation, resting energy expenditure and results on a behavioral questionnaire for the subject; (c) populating a predictive machine learning model with the obesity analyte signature of step (a) and results from step (b) for the subject; and (d) utilizing the predictive machine learning model to predict an obesity phenotype of the subject suffering from obesity. In some cases, the predictive machine learning model is a multivariate logistic regression machine learning model. The multivariate logistic regression can be a least absolute shrinkage and selection operator (LASSO) regression. In some cases, the obesity phenotype is selected from the group consisting of abnormal satiation (hungry brain), abnormal satiety (hungry gut); hedonic eating (emotional hunger) and slow metabolism (slow burn). In some cases, utilization of the predictive machine learning model predicts the obesity phenotype of the mammal suffering from obesity with a sensitivity of about, at most, at least 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. In some cases, utilization of the predictive machine learning model predicts the obesity phenotype of the mammal suffering from obesity with a specificity of about, at most, at least 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. In some cases, utilization of the predictive machine learning model predicts the obesity phenotype of the mammal suffering from obesity with a positive predictive value of about, at most, at least 50%, 51%, 52%, 53%, 54%, 55%, 56%, 57%, 58%, 59%, 60%, 61%, 62%, 63%, 64%, 65%, 66%, 67%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98% or 99%. Satiety can be determined by measuring gastric emptying. Gastric emptying can be measured using any method known in the art such as, for example, scintigraphy and can be represented as GE T1/4 or GE T1/2. Satiation can be determined by measuring calories to fullness or the amount of calories (e.g., kcal) consumed at an ad libitum meal. Resting energy expenditure can be measured using any method known in the art such as, for example, indirect calorimetry and can be represented as kcal/24 hrs. The behavioral questionnaire can be any questionnaire known in the art for assessing anxiety and/or emotional eating, for example, the Hospital Anxiety and Depression Scale (HADS) questionnaire.

### Multi-Omic Data

As described previously herein, any of the machine learning based methods provided herein for determining an individual's obesity phenotype can utilize or integrate multi-omic data. The multi-omic data utilized or integrated into a machine learning obesity phenotyping method provided herein can comprise genetic or non-genetic multi-omic data obtained from an individual or a sample obtained from an individual as required by said machine learning obesity phenotyping method provided herein. The multi-omic data can be selected from the group consisting of metabolomic data, genomic data, proteomic data, peptidomic data and any combination thereof. In some cases, the multi-omic data obtained for an individual or a sample obtained from an individual represents an obesity analyte signature for that individual.

An obesity analyte signature can include any appropriate analyte. Examples of analytes that can be included in an obesity analyte signature described herein include, without limitation, DNA, RNA, proteins, peptides, metabolites, hormones, and exogenous compounds (e.g., medications). In some cases, the obesity analyte signature can be obtained by detecting the presence, absence, or level of one or more metabolites, detecting the presence, or absence, or level one or more peptides (e.g., gastrointestinal peptides), and/or detecting the presence or absence of one or more single nucleotide polymorphisms (SNPs). An obesity analyte signature can be evaluated using any appropriate methods. For example, metabolomics, genomics, microbiome, proteomic, peptidomics, and behavioral questionnaires can be used to evaluate and/or identify an obesity analyte signature described herein.

A metabolite can be any metabolite that is associated with obesity. In some cases, a metabolite can be an amino-compound. In some cases, a metabolite can be a neurotransmitter. In some cases, a metabolite can be a fatty acid (e.g., a short chain fatty acid). In some cases, a metabolite can be an amino compound. In some cases, a metabolite can be a bile acid. Examples of metabolites that can be used to determine the obesity analyte signature in a sample (e.g., in a sample obtained from an obese mammal) include, without limitation. 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine. DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

A gastrointestinal peptide can be any gastrointestinal peptide that is associated with obesity. In some cases, a gastrointestinal peptide can be a peptide hormone. In some cases, a gastrointestinal peptide can be released from gastrointestinal cells in response to feeding. Examples of gastrointestinal peptides that can be used to determine the obesity analyte signature in a sample (e.g., in a sample obtained from an obese mammal) include, without limitation, ghrelin, peptide tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, and pancreatic polypeptide.

A SNP can be any SNP that is associated with obesity. A SNP can be in a coding sequence (e.g., in a gene) or a non-coding sequence. For example, in cases where a SNP is in a coding sequence, the coding sequence can be any appropriate coding sequence. Examples of coding sequences that a SNP associated with obesity can be in or near include, without limitation, ADRA2A, ADRA2C, BDNF, CADM2, GNB3, FTO, MC4R, TCF7L2, 5-HTTLPR, HTR2C, UCP2, UCP3, GPBAR1, NR1H4, FAIM2, FANCL, FGFR4, PYY, GLP-1, CCK, leptin, adiponectin, neurotensin, ghrelin, GLP-1 receptor, GOAT, GPRC5B, GNPDA2, DPP4, POMC, NPY, AGRP, SERT, BDNF, SLC6A4, DRD2, LEP, LEPR, UCP1, KLF14, NPC1, LYPLAL1, ADRB2, ADRB3, BBS1, ACSL6, ADARB2, ADCY8, ADH1B, AJAP1, ATP2C2, ATP6VOD2, C21orf7, CAMKMT, CAP2, CASC4, CD48, CDC42SE2, CDYL, CES5AP1, CLMN, CNPY4, COL19A1, COL27A1, COL4A3, CORO1C, CPZ, CTIF, DAAM2, DCHS2, DOCKS, EGFLAM, FAM125B, FAM71E2, FRMD3, GALNTL4. GLT1D1, HHAT, HOXB5, KCTD15, KRT23, LEP, LEPR, LHPP, LINC00578, LINC00620, LINGO1, LIPC, LOC100128714, LOC100287160, LOC100289473, LOC100293612|L1NC00620, LOC100506869, LOC100507053, LOC100507053|ADH1A, LOC100507053|ADH, LOC100507443, LOC100996571|CYYR1, LOC152225, LOC255130, LPAR1, LUZP2, MCM7, MICAL3, MMS19, MTCH2, MYBPC1, NEGR1, NR2F2-AS1, NSMCE2, NTN1, NUDT3, O3FAR1, OAZ2, OLFM4, OSBP2, P4HA2, PADI1, PARD3B, PARK2, PCDH15, PCSK1, PIEZO2. PKIB, PRH1-PRR4, PTBP2, PTPRD, RALGPS1|ANGPTL2, RPS24P10, RTN4RL1, RYR2, SCN2A, SEC16B, SEMA3C, SEMA5A, SFMBT2, SGCG, SH2B, SIM1, SLC22A15, SLC2A2, SLCO1B1, SMOC2, SNCAIP, SNX18, NCOA1 (SRC1), SRRM4, SUSD1, TBC1D16, TCERG1L, TENM3, TJP3, TLL1, TMEM9B, TMEM18, TNNI3K, TPM1, VTI1A, VWF, WWOX, WWTR1, ZFYVE28, ZNF3, ZNF609, and ZSCAN21. Examples of SNPS that can be used to determine the obesity analyte signature in a sample (e.g., in a sample obtained from an obese mammal) include, without limitation, rs657452, rs11583200, rs2820292, rs11126666, rs11688816, rs1528435, rs7599312, rs6804842, rs2365389, rs3849570, rs16851483, rs17001654, rs11727676, rs2033529, rs9400239, rs13191362, rs167827, rs2245368, rs2033732, rs4740619, rs6477694, rs1928295, rs10733682, rs7899106, rs17094222, rs11191560, rs7903146, rs2176598, rs12286929, rs11057405, rs10132280, rs12885454, rs3736485, rs758747, rs2650492, rs9925964, rs1,000940, rs1808579, rs7243357, rs17724992, rs977747, rs1460676, rs17203016, rs13201877, rs1441264, rs7164727, rs2080454, rs9914578, rs2836754, rs492400, rs16907751, rs9374842, rs9641123, rs9540493, rs4787491, rs6465468, rs7239883, rs3101336, rs12566985, rs12401738, rs11165643, rs17024393, rs543874, rs13021737, rs10182181, rs1016287, rs2121279, rs13078960, rs1516725, rs10938397, rs13107325, rs2112347, rs205262, rs2207139, rs17405819, rs10968576, rs4256980, rs11030104, rs3817334, rs7138803, rs12016871, rs12429545, rs11847697, rs7141420, rs16951275, rs12446632, rs3888190, rs1558902, rs12940622, rs6567160, rs29941, rs2075650, rs2287019, rs3810291, rs7715256, rs2176040, rs6091540, rs1800544, Ins-Del-322, rs5443, rs1129649, rs1047776, rs9939609, rs17782313, rs7903146, rs4795541, rs3813929, rs518147, rs1414334, rs659366, −3474, rs2075577, rs15763, rs1626521, rs11554825, rs4764980, rs434434, rs351855, and rs2234888.Examples of SNPS that can be used to determine the obesity analyte signature in a sample (e.g., in a sample obtained from an obese mammal) include, without limitation, can include any or all of the SNPs associated with an obesity analyte signature as described in WO2022246203A1, which is herein incorporated by reference in its entirety. For example, SNPS that can be used to determine the obesity analyte signature in a sample (e.g., in a sample obtained from an obese mammal) include, without limitation, rs1664232, rs11118997, rs9342434, rs2335852, rs11020655, rs1885034, rs7277175, rs6923761, rs7903146, rs17782313, rs3813929, rs1047776 and any combination thereof.

An obesity analyte signature described herein can include any appropriate combination of analytes. For example, when an obesity analyte signature includes 14 analytes, the analytes can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine, ghrelin, and PYY. For example, when an obesity analyte signature includes 9 analytes, the analytes can include HTR2C, GNB3, FTO, isocaproic, beta-aminoisobutyric-acid, butyric, allo-isoleucine, tryptophan, and glutamine.

Any appropriate method can be used to detect the presence, absence, or level of an obesity analyte within a sample. For example, mass spectrometry (e.g., triple-stage quadrupole mass spectrometry coupled with ultra-performance liquid chromatography (UPLC)), radioimmunoassays, enzyme-linked immunosorbent assays, sequencing techniques (e.g., PCR-based sequencing techniques), and/or restriction fragment length polymorphism (RFLP) can be used to determine the presence, absence, or level of one or more analytes in a sample.

In some cases, identifying the obesity phenotype can include obtaining results from one or more questionnaires. A questionnaire can be associated with obesity. In some cases, a questionnaire can be answered the time of the assessment. In some cases, a questionnaire can be answered prior to the time of assessment. For example, when a questionnaire is answered prior to the time of the assessment, the questionnaire results can be obtained by reviewing a patient history (e.g., a medical chart). A questionnaire can be a behavioral questionnaire (e.g., psychological welfare questionnaires, questionnaire for assessing anxiety and/or depression, alcohol use questionnaires, eating behavior questionnaires, body image questionnaires, physical activity level questionnaire, and weight management questionnaires). Examples of questionnaires that can be used to determine the obesity phenotype of a mammal (e.g., an obese mammal) include, without limitation, The Hospital Anxiety and Depression Scale (HADS) questionnaire. The Hospital Anxiety and Depression Inventory questionnaire, The Questionnaire on Eating and Weight Patterns, The Weight Efficacy Life-Style (WEL) Questionnaire, Three-Factor Eating Questionnaire (TFEQ), and The Multidimensional Body-Self Relations Questionnaire. For example, a questionnaire can be a HADS questionnaire.

In some cases, an obesity analyte signature can include the presence of serotonin, glutamine, isocaproic, allo-isoleucine, hydroxyproline, beta-aminoisobutyric-acid, alanine, hexanoic, tyrosine, and PYY. For example, a hungry brain (i.e., abnormal satiation) obesity phenotype can have an obesity analyte signature that includes the presence of serotonin, glutamine, isocaproic, allo-isoleucine, hydroxyproline, beta-aminoisobutyric-acid, alanine, hexanoic, tyrosine, and PYY. For example, a hungry brain (i.e., abnormal satiation) obesity phenotype can have an obesity analyte signature that has an absence of (e.g., lacks the presence of) 1-methylhistine, gamma-amino-n-butyric-acid, phenylalanine, ghrelin, and includes a HADS questionnaire result that does not indicate an anxiety subscale (HADS-A; e.g., includes a HADS-A questionnaire result).

In some cases, an obesity analyte signature can include the presence of 1-methylhistine, allo-isoleucine, hydroxyproline, beta-aminoisobutyric-acid, alanine, and phenylalanine. For example, a hungry gut (i.e., abnormal satiety) obesity phenotype can have an obesity analyte signature that includes the presence of 1-methylhistine, allo-isoleucine, hydroxyproline, beta-aminoisobutyric-acid, alanine, and phenylalanine. For example, a hungry gut (i.e., abnormal satiety) obesity phenotype can have an obesity analyte signature that has an absence of (e.g., lacks the presence of) serotonin, glutamine, gamma-amino-n-butyric-acid, isocaproic, hexanoic, tyrosine, ghrelin, PYY, and does not include a HADS questionnaire result that indicates an anxiety subscale (e.g., does not include a HADS-A questionnaire result).

In some cases, an obesity analyte signature can include the presence of serotonin and can include a HADS-A questionnaire. For example, an emotional or hedonic eating (e.g., behavioral eating) obesity phenotype can have an obesity analyte signature that includes serotonin and includes a HADS-A questionnaire result. For example, an emotional or hedonic eating (e.g., behavioral eating) obesity phenotype can have an obesity analyte signature that has an absence of (e.g., lacks the presence of) 1-methylhistine, glutamine, gamma-amino-n-butyric-acid, isocaproic, allo-isoleucine, hydroxyproline, beta-aminoisobutyric-acid, alanine, hexanoic, tyrosine, phenylalanine, ghrelin, and PYY.

In some cases, an obesity analyte signature can include the presence of 1-methylhistine, glutamine, gamma-amino-n-butyric-acid, isocaproic, allo-isoleucine, beta-aminoisobutyric-acid, alanine, hexanoic, tyrosine, phenylalanine, PYY, and includes a HADS-A questionnaire result. For example, a large fasting gastric volume obesity phenotype can have an obesity analyte signature that includes 1-methylhistine, glutamine, gamma-amino-n-butyric-acid, isocaproic, allo-isoleucine, beta-aminoisobutyric-acid, alanine, hexanoic, tyrosine, phenylalanine, PYY, and includes a HADS-A questionnaire result. For example, a large fasting gastric volume obesity phenotype can have an obesity analyte signature that has an absence of (e.g., lacks the presence of) serotonin, hydroxyproline, and ghrelin.

In some cases, an obesity analyte signature can include the presence of serotonin, beta-aminoisobutyric-acid, alanine, hexanoic, phenylalanine, and includes a HADS-A questionnaire. For example, an obesity phenotype that is mixed can have an obesity analyte signature that includes the presence of serotonin, beta-aminoisobutyric-acid, alanine, hexanoic, phenylalanine, and includes a HADS-A questionnaire result. For example, an obesity phenotype that is mixed can have an obesity analyte signature that has an absence of (e.g., lacks the presence of) 1-methylhistine, glutamine, gamma-amino-n-butyric-acid, isocaproic, allo-isoleucine, and hydroxyproline.

In some cases, an obesity analyte signature can include the presence of 1-methylhistine, serotonin, glutamine, gamma-amino-n-butyric-acid, isocaproic, allo-isoleucine, alanine, tyrosine, ghrelin, PYY, and includes a HADS-A questionnaire result. For example, a low resting energy expenditure (e.g., slow burn) obesity phenotype can have an obesity analyte signature that includes the presence of 1-methylhistine, serotonin, glutamine, gamma-amino-n-butyric-acid, isocaproic, allo-isoleucine, alanine, tyrosine, ghrelin, PYY, and includes a HADS-A questionnaire result. For example, a low resting energy expenditure (e.g., slow burn) obesity phenotype can have an obesity analyte signature that has an absence of (e.g., lacks the presence of) hydroxyproline, beta-aminoisobutyric-acid, hexanoic, and phenylalanine.

In some cases, identifying the obesity phenotype also can include identifying one or more additional variables and/or one or more additional assessments. For example, identifying the obesity phenotype also can include assessing the microbiome of a mammal (e.g., an obese mammal). For example, identifying the obesity phenotype also can include assessing leptin levels. For example, identifying the obesity phenotype also can include assessing the metabolome of a mammal (e.g., an obese mammal). For example, identifying the obesity phenotype also can include assessing the genome of a mammal (e.g., an obese mammal). For example, identifying the obesity phenotype also can include assessing the proteome of a mammal (e.g., an obese mammal). For example, identifying the obesity phenotype also can include assessing the peptidome of a mammal (e.g., an obese mammal).

### Clinical Uses

Once the obesity phenotype of the mammal has been identified using any of the methods provided herein, the obesity phenotype can be used to select a treatment option for the mammal. In some cases, determining the obesity phenotype of a subject using the methods provided herein can be used to identify the subject as being responsive or non-responsive to a particular intervention (e.g., pharmacological intervention. For example, use of the M-GRS and FPT methods provided herein can be used to identify that the subject possesses a hungry brain phenotype that can serve to identify or select the subject as a non-responder to treatment with a GLP-1 agonist or a responder to phentermine-topiramate pharmacotherapy. For example, once a mammal is identified as being responsive to one or more interventions (e.g., pharmacological intervention, surgical intervention, weight loss device, diet intervention, lifestyle intervention, behavior intervention, and/or microbiome intervention) based, at least in part, on an obesity phenotype, which is based, at least in part, on an obesity analyte signature in the sample, the mammal can be administered or instructed to self-administer one or more interventions. The GLP-1 agonist can be any agent known in the art whose mechanism of action comprises activating the GLP-1 receptor. GLP-1 agonists for use in a method and/or system provided herein can include, but are not limited to, exenatide, liraglutide, albiglutide, dulaglutide, lixisenatide, semaglutide, tirzepatide or any combination thereof.

When treating obesity in a subject as described herein, the intervention can be effective to reduce the weight, reduce the waist circumference and/or slow or prevent weight gain of the subject. For example, treatment described herein can be effective to reduce the weight (e.g., the total body weight) of a subject with obesity by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. Treatment described herein can be effective to reduce the weight (e.g., the total body weight) of a with obesity subject by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. Treatment described herein can be effective to reduce the weight (e.g., the total body weight) of a subject with obesity by at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. In some cases, the intervention described herein can be effective to reduce the weight (e.g., the total body weight) of a subject with obesity by at least 3%, at least 5%, at least 8%, at least 10%, at least 12%, at least 15%, at least 18%, at least 20%, at least 22%, at least 25%, at least 28%, at least 30%, at least 33%, at least 36%, at least 39%, or at least 40%). For example, the intervention described herein can be effective to reduce the weight (e.g., the total body weight) of a subject with obesity by from about 3% to about 40% (e.g., from about 3% to about 35%, from about 3% to about 30%, from about 3% to about 25%, from about 3% to about 20%, from about 3% to about 15%, from about 3% to about 10%, from about 3% to about 5%, from about 5% to about 40%, from about 10% to about 40%, from about 15% to about 40%, from about 20% to about 40%, from about 25% to about 40%, from about 35% to about 40%, from about 5% to about 35%, from about 10% to about 30%, from about 15% to about 25%, or from about 18% to about 22%). For example, the intervention described herein can be effective to reduce the weight (e.g., the total body weight) of a subject with obesity by from about 3 kg to about 100 kg (e.g., about 5 kg to about 100 kg, about 8 kg to about 100 kg, about 10 kg to about 100 kg, about 15 kg to about 100 kg, about 20 kg to about 100 kg, about 30 kg to about 100 kg, about 40 kg to about 100 kg, about 50 kg to about 100 kg, about 60 kg to about 100 kg, about 70 kg to about 100 kg, about 80 kg to about 100 kg, about 90 kg to about 100 kg, about 3 kg to about 90 kg, about 3 kg to about 80 kg, about 3 kg to about 70 kg, about 3 kg to about 60 kg, about 3 kg to about 50 kg, about 3 kg to about 40 kg, about 3 kg to about 30 kg, about 3 kg to about 20 kg, about 3 kg to about 10 kg, about 5 kg to about 90 kg, about 10 kg to about 75 kg, about 15 kg to about 50 kg, about 20 kg to about 40 kg, or about 25 kg to about 30 kg). For example, the intervention described herein can be effective to reduce the waist circumference of a subject with obesity by from about 1 inches to about 10 inches (e.g., about 1 inches to about 9 inches, about 1 inches to about 8 inches, about 1 inches to about 7 inches, about 1 inches to about 6 inches, about 1 inches to about 5 inches, about 1 inches to about 4 inches, about 1 inches to about 3 inches, about 1 inches to about 2 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 9 inches to about 10 inches, about 2 inches to about 9 inches, about 3 inches to about 8 inches, about 4 inches to about 7 inches, or about 5 inches to about 7 inches. Administration of any intervention provided herein to a subject determined to possess an obesity phenotype using a method described herein can result in a reduction of the waist circumference of the subject with obesity of at least 1, 2, 3, 4, 5, 6, 7, 8, 9 or 10 inches. Administration of any intervention provided herein to a subject determined to possess an obesity phenotype using a method described herein can result in a reduction of the waist circumference of the subject with obesity of at most 1, 2, 3, 4, 5, 6, 7, 8, 9 or 10 inches. Administration of any intervention provided herein to a subject determined to possess an obesity phenotype using a method described herein can result in a reduction of the waist circumference of the subject with obesity of about 1, 2, 3, 4, 5, 6, 7, 8, 9 or 10 inches.

For example, any treatment described herein can be effective to result in a total body weight lost (TBWL) of the subject with obesity of at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Any treatment described herein can be effective to result in a total body weight lost (TBWL) of the subject with obesity of at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Any treatment described herein can be effective to result in a total body weight lost (TBWL) of the subject with obesity of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Any treatment described herein can be effective to result in a total body weight lost (TBWL) of the subject with obesity of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Any treatment described herein can be effective to result in a total body weight lost (TBWL) of the subject with obesity of from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80%. In one embodiment, administration of any intervention provided herein to a subject determined to possess an obesity phenotype using a method described herein can result in a total body weight lost (TBWL) of the subject with obesity of from 2% to 8%. In one embodiment, administration of any intervention provided herein to a subject determined to possess an obesity phenotype using a method described herein can result in a total body weight lost (TBWL) of the subject with obesity of at least 3%. In one embodiment, administration of any intervention provided herein to a subject determined to possess an obesity phenotype using a method described herein can result in a total body weight lost (TBWL) of the subject with obesity of at least 6%.

Administration of any intervention provided herein to a subject determined to possess an obesity phenotype using a method described herein can result in a total body weight lost (TBWL) of the subject with obesity of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of any intervention provided herein to a subject determined to possess an obesity phenotype using a method described herein can result in a total body weight lost (TBWL) of the subject with obesity of at most 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of any intervention provided herein to a subject determined to possess an obesity phenotype using a method described herein can result in a total body weight lost (TBWL) of the subject with obesity of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of any intervention provided herein to a subject determined to possess an obesity phenotype using a method described herein can result in a total body weight lost (TBWL) of the subject with obesity of from about 3 kg to about 100 kg, about 5 kg to about 100 kg, about 8 kg to about 100 kg, about 10 kg to about 100 kg, about 15 kg to about 100 kg, about 20 kg to about 100 kg, about 30 kg to about 100 kg, about 40 kg to about 100 kg, about 50 kg to about 100 kg, about 60 kg to about 100 kg, about 70 kg to about 100 kg, about 80 kg to about 100 kg, about 90 kg to about 100 kg, about 3 kg to about 90 kg, about 3 kg to about 80 kg, about 3 kg to about 70 kg, about 3 kg to about 60 kg, about 3 kg to about 50 kg, about 3 kg to about 40 kg, about 3 kg to about 30 kg, about 3 kg to about 20 kg, about 3 kg to about 10 kg, about 5 kg to about 90 kg, about 10 kg to about 75 kg, about 15 kg to about 50 kg, about 20 kg to about 40 kg, or about 25 kg to about 30 kg.

Administration of any intervention provided herein to a subject determined to possess an obesity phenotype using a method described herein can result in a decrease in the waist circumference of the subject with obesity of at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of any intervention provided herein to a subject determined to possess an obesity phenotype using a method described herein can result in a decrease in the waist circumference of the subject with obesity of at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of any intervention provided herein to a subject determined to possess an obesity phenotype using a method described herein can result in a decrease in the waist circumference of the subject with obesity of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of any intervention provided herein to a subject determined to possess an obesity phenotype using a method described herein can result in a decrease in the waist circumference of the subject with obesity of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of any intervention provided herein to a subject determined to possess an obesity phenotype using a method described herein can result in a decrease in the waist circumference of the subject with obesity of from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80%.

In some cases, the change or alteration in the body weight (e.g., total body weight lost (TBWL)) of a subject with an obesity phenotype as described herein, the waist circumference of the subject, the characteristics that define the obesity phenotype of the subject (e.g., CTS, gastric emptying, resting energy expenditure (REE), indication of anxiety on HADS, etc.) or any combination thereof that results from administration of any intervention provided herein to the subject results persists for a defined or specified amount of time. The defined or specified amount of time can be at least 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. The defined or specified amount of time can be at most 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. The defined or specified amount of time can be about 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. In some cases, the defined or specified amount of time can be at least 15, 20, 25, 30, 35, 40, 45, 50, 55, 60, 65 or 70 years.

Individualized pharmacological interventions for the treatment of obesity (e.g., based on the obesity phenotypes assessed using a method described herein) can include any one or more (e.g., 1, 2, 3, 4, 5, 6, or more) pharmacotherapies (e.g., individualized pharmacotherapies). A pharmacotherapy can include any appropriate pharmacotherapy. In some cases, a pharmacotherapy can be an obesity pharmacotherapy. In some cases, a pharmacotherapy can be an appetite suppressant. In some cases, a pharmacotherapy can be an anticonvulsant. In some cases, a pharmacotherapy can be a GLP-1 agonist. The GLP-1 agonist can be any agent known in the art whose mechanism of action comprises activating the GLP-1 receptor. GLP-1 agonists for use in a method and/or system provided herein can include, but are not limited to, exenatide, liraglutide, albiglutide, dulaglutide, lixisenatide, semaglutide, tirzepatide or any combination thereof. In some cases, a pharmacotherapy can be an antidepressant. In some cases, a pharmacotherapy can be an opioid antagonist. In some cases, a pharmacotherapy can be a controlled release pharmacotherapy. For example, a controlled release pharmacotherapy can be an extended release (ER) and/or a slow release (SR) pharmacotherapy. In some cases, a pharmacotherapy can be a lipase inhibitor. In some cases, a pharmacotherapy can be a DPP4 inhibitor. In some cases, a pharmacotherapy can be a SGLT2 inhibitor. In some cases, a pharmacotherapy can be a dietary supplement. Examples of pharmacotherapies that can be used in an individualized pharmacological intervention as described herein include, without limitation, orlistat, phentermine, topiramate, lorcaserin, naltrexone, bupropion, liraglutide, exenatide, metformin, pramlintide, Januvia, canagliflozin, dexamphetamines, prebiotics, probiotics, postbiotics, Ginkgo biloba, and combinations thereof. For example, combination pharmacological interventions for the treatment of obesity (e.g., based on the obesity phenotypes as described herein) can include phentermine-topiramate ER, naltrexone-bupropion SR, phentermine-lorcaserin, lorcaserin-liraglutide, and lorcaserin-januvia. In some cases, a pharmacotherapy can be administered as described elsewhere (see, e.g., Sjostrom et al., 1998 Lancet 352:167-72; Hollander et al., 1998 Diabetes Care 21:1288-94; Davidson et al., 1999 JAMA 281:235-42; Gadde et al., 2011 Lancet 377:1341-52; Smith et al., 2010 New Engl. J. Med. 363:245-256; Apovian et al., 2013 Obesity 21:935-43; Pi-Sunyer et al., 2015 New Engl. J. Med. 373:11-22; and Acosta et al., 2015 Clin Gastroenterol Hepatol. 13:2312-9).

When a mammal is identified as having an obesity phenotype using a machine learning method provided herein (e.g., ML-driven, full phenotype method or adaptive ML-GRS method) that is responsive to treatment with one or more pharmacotherapies, the mammal can be administered or instructed to self-administer one or more pharmacotherapies. In some cases, when a mammal is identified as having a hungry gut (e.g., abnormal satiety) phenotype, based, at least in part, on an obesity analyte signature, the mammal can be administered or instructed to self-administer one or more a GLP-1 agonists (e.g., liraglutide or semaglutide) to treat the obesity. In some cases, when a mammal is identified as having a hungry brain (e.g., abnormal satiation) phenotype, based, at least in part, on a hungry brain ML-GRS provided herein, the mammal can be administered or instructed to self-administer phentermine-topiramate pharmacotherapy and/or lorcaserin pharmacotherapy to treat the obesity. In some cases, when a mammal is identified as having a hungry brain (e.g., abnormal satiation) phenotype, based, at least in part, on a hungry brain ML-GRS provided herein, the mammal can be administered or instructed to self-administer a pharmacotherapy that is not a GLP-1 agonist. In other words, a subject who is diagnosed or determined to possess a hungry brain (i.e., abnormal satiation) phenotype can be classified or selected as a non-responder to GLP-1 agonist treatment. In some cases, when a mammal is identified as having a slow burn (e.g., energy expenditure) phenotype, based, at least in part, on a slow burn ML-GRS provided herein, the mammal can be administered or instructed to self-administer phentermine to treat the obesity. In some cases, when a mammal is identified as having a hedonic eating (e.g., behavioral/psychological eating) phenotype, based, at least in part, on a hedonic eating ML-GRS provided herein, the mammal can be administered or instructed to self-administer naltrexone/bupropion to treat the obesity. The GLP-1 agonist can be any agent known in the art whose mechanism of action comprises activating the GLP-1 receptor. GLP-1 agonists for use in a method and/or system provided herein can include, but are not limited to, exenatide, liraglutide, albiglutide, dulaglutide, lixisenatide, semaglutide, tirzepatide or any combination thereof.

In some cases, one or more pharmacotherapies described herein can be administered to an obese mammal as a combination therapy with one or more additional agents/therapies used to treat obesity. For example, a combination therapy used to treat an obese mammal (e.g., a human with obesity) can include administering to the mammal one or more pharmacotherapies described herein and one or more obesity treatments such as weight-loss surgeries (e.g., gastric bypass surgery, laparoscopic adjustable gastric banding (LAGB), biliopancreatic diversion with duodenal switch, and a gastric sleeve), vagal nerve blockade, endoscopic devices (e.g., intragastric balloons or endoliners, magnets), endoscopic sleeve gastroplasty, and/or gastric or duodenal ablations. For example, a combination therapy used to treat an obese mammal (e.g., a human with obesity) can include administering to the mammal one or more pharmacotherapies described herein and one or more obesity therapies such as exercise modifications (e.g., increased physical activity, change in type of physical activity, timing and frequency of physical activity), dietary modifications (e.g., reduced-calorie diet), behavioral modifications, lifestyle intervention, commercial weight loss programs, wellness programs, and/or wellness devices (e.g. dietary tracking devices and/or physical activity tracking devices). In cases where one or more pharmacotherapies described herein are used in combination with one or more additional agents/therapies used to treat obesity, the one or more additional agents/therapies used to treat obesity can be administered/performed at the same time or independently. For example, the one or more pharmacotherapies described herein can be administered first, and the one or more additional agents/therapies used to treat obesity can be administered/performed second, or vice versa.

In some cases, efficacy of any intervention provided herein can be ascertained by a change or alteration in the body weight (e.g., total body weight lost (TBWL)) of the subject as provided herein, waist circumference, the characteristics that define the hungry brain phenotype or any combination thereof. For example, a subject determined to possess a hungry brain phenotype displayed a decrease in the number of calories consumed to reach fullness following administration of any intervention provided herein as compared to the number of calories consumed to reach fullness for the same subject prior to administration of the intervention. The number of calories consumed to reach fullness following administration of any intervention described herein can be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the intervention. The number of calories consumed to reach fullness following administration of any intervention described herein can be at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the intervention. The number of calories consumed to reach fullness following administration of any intervention described herein can be about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the intervention. The number of calories consumed to reach fullness following administration of any intervention described herein can be from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% of the number of calories consumed to reach fullness for the same subject prior to administration of the intervention. The number of kcal consumed to reach fullness following administration of any intervention described herein can be reduced by at least 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the intervention. The number of kcal consumed to reach fullness following administration of any intervention described herein can be reduced by at most 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the intervention. The number of kcal consumed to reach fullness following administration of the intervention described herein can be reduced by about 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the intervention. The fullness both pre- and -post intervention administration, the fullness can be a maximal fullness (MTV) or normal or usual fullness (VTF). The ‘maximal’ fullness (MTV) or ‘usual’ fullness (VTF) can be as measured in a nutrient drink test or to mixed meal (solids) in an ad libitum buffet meal.

In some cases, efficacy of any intervention provided herein can be ascertained by a change or alteration in the body weight (e.g., total body weight lost (TBWL)) of the subject as provided herein, waist circumference, the characteristics that define the hungry gut phenotype or any combination thereof. For example, a subject determined to possess a hungry gut phenotype displayed a decrease in gastric emptying (e.g., gastric half-emptying time (GE t_{1/2})) following administration of any intervention provided herein as compared to the gastric emptying (e.g., gastric half-emptying time (GE t_{1/2})) for the same subject prior to administration of the intervention. Gastric emptying in a subject determined to possess a hungry gut phenotype following administration of any intervention described herein can be decreased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the intervention. Gastric emptying in a subject determined to possess a hungry gut phenotype following administration of any intervention described herein can be decreased by at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the intervention. Gastric emptying in a subject determined to possess a hungry gut phenotype following administration of any intervention described herein can be decreased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the intervention. Gastric emptying in a subject determined to possess a hungry gut phenotype following administration of any intervention described herein can be decreased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the gastric emptying for the same subject prior to administration of the intervention.

In some cases, efficacy of any intervention provided herein can be ascertained by a change or alteration in the body weight (e.g., total body weight lost (TBWL)) of the subject as provided herein, waist circumference, the characteristics that define the emotional hunger phenotype or any combination thereof. For example, a subject determined to possess an emotional hunger phenotype displayed a change in the hospital anxiety and depression score (HADS) questionnaire following administration of any intervention provided herein as compared to the same subject prior to administration of the intervention such that the HADS questionnaire result does not indicate a positive an anxiety component following administration of the intervention. In some cases, the change in the hospital anxiety and depression score (HADS) questionnaire following administration of the intervention can be a decrease in the score on the HADS questionnaire by about, at most or at least 1, 2, 3 or 4 as compared to the same subject prior to administration of the intervention such that the HADS questionnaire result.

In some cases, efficacy of any intervention provided herein can be ascertained by a change or alteration in the body weight (e.g., total body weight lost (TBWL)) of the subject as provided herein, waist circumference, the characteristics that define the slow burn phenotype (e.g., lean muscle mass and/or REE) or any combination thereof. For example, a subject determined to possess a slow burn phenotype displayed an increase in resting energy expenditure ((REE); indirect calorimetry; either % or kcal/day) following administration of any intervention provided herein as compared to the REE (either % or kcal/day) for the same subject prior to administration of the intervention. REE in a subject determined to possess a slow burn phenotype following administration of any intervention described herein can be increased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the intervention. REE in a subject determined to possess a slow burn phenotype following administration of any intervention described herein can be increased by at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the intervention. REE in a subject determined to possess a slow burn phenotype following administration of any intervention described herein can be increased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the intervention. REE in a subject determined to possess a slow burn phenotype following administration of any intervention described herein can be increased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the REE for the same subject prior to administration of the intervention. In some cases, a subject determined to possess a slow burn phenotype displayed an increase in lean muscle mass following administration of any intervention provided herein as compared to the lean muscle mass for the same subject prior to administration of the intervention. Lean muscle mass in a subject determined to possess a slow burn phenotype following administration of any intervention described herein can be increased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the lean muscle mass for the same subject prior to administration of the intervention. Lean muscle mass in a subject determined to possess a slow burn phenotype following administration of any intervention described herein can be increased by at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the lean muscle mass for the same subject prior to administration of the intervention. Lean muscle mass in a subject determined to possess a slow burn phenotype following administration of any intervention described herein can be increased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the lean muscle mass for the same subject prior to administration of the intervention. Lean muscle mass in a subject determined to possess a slow burn phenotype following administration of any intervention described herein can be increased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the lean muscle mass for the same subject prior to administration of the intervention.

### Systems

A computer implemented method described herein ma, be performed on a single computing machine, a virtual machine, a distributed computing system that includes multiples nodes of computing machines, or any other suitable arrangement of computing devices.

In one embodiment, any of the methods provided herein for determining the obesity phenotype of a subject suffering from obesity may be perform by a system comprising (a) one or more processors; (b) one or more memories or computer-readable medium operatively coupled to at least one of the one or more processors and having instructions stored thereon that, when executed by at least one of the one or more processors, cause the system to performing a method provided herein for determining the obesity phenotype (i.e., ML, FPT method or adaptive, contribution factor based ML-GRS method); and c) one or more instruments in communication with at least one of the one or more processors, wherein the instruments, upon receipt of instructions sent by the at least one of the one or more processors, perform the method provided herein for determining the obesity phenotype (i.e., ML, FPT method or adaptive, contribution factor based ML-GRS method). The one or more processors can be one or more computers and coupled together can form a computer system for implementing any method provided herein.

In some embodiments, the computing system operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of the computer system may correspond to any software, hardware, or combined components including but not limited to, a client device, a computing server, and various engines, interfaces, terminals, and machines shown.

By was of example, the computer system mac comprise a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions that specify actions to be taken by that machine. Further, the term “machine” and “computer” may also be taken to include any collection of machines that individually or jointly execute instructions to perform any one or more of the methodologies discussed herein.

Instructions can be any directions, commands, or orders that may be stored in different forms, such as equipment-readable instructions, programming instructions including source code, and other communication signals and orders. Instructions may be used in a general sense and are not limited to machine-readable codes.

One and more methods described herein improve the operation speed of the processors used in a system provided herein and reduces the space required for the memory. For example, the machine learning methods described herein can reduce the complexity of the computation of the processors by applying one or more novel techniques that simplify the steps in training, reaching convergence, and generating results of the processors. The algorithms described herein also reduces the size of the models and datasets to reduce the storage space requirement for memory.

The performance of certain of the operations may be distributed among the more than one processors, not only residing within a single machine, but deployed across a number of machines. In some example embodiments, the one or more processors or processor-implemented engines may be located in a single geographic location (e.g., within a home environment, an office environment, or a server farm). In other example embodiments, the one or more processors or processor-implemented engines may be distributed across a number of geographic locations. Even though in the specification or the claims may refer some processes to be performed by a processor, this should be construed to include a joint operation of multiple distributed processors.

The term “computer-readable medium” should be taken to include a single medium or multiple media (e.g., a centralized or distributed database, or associated caches and servers) able to store instructions. The computer-readable medium ma, include any medium that is capable of storing instructions for execution by the processors and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave and is, thus non-transitory.

### EXAMPLES

The present disclosure is further illustrated by reference to the following Examples. However, it should be noted that these Examples, like the embodiments described above, are illustrative and are not to be construed as restricting the scope of the invention in any way.

### Example 1—Abnormal Satiation as a Predictor for GLP-1 Receptor Agonist Treatment

### Background

Obesity is a disease of energy imbalance, where food intake regulation takes a central role. The food intake cycle can be divided in stages that are hunger, satiation and satiety. Multi-omics biomarkers in obesity can provide insight into its etiology and pathophysiology links with chronic illnesses, which may eventually help approach obesity more efficiently and individually. (Aleksandrova et al. 2020). These omics-based biomarkers also allow for the analysis of multiple factors for prediction at the individual level. These high-throughput profiling technologies have explored the connections between a high body mass index or adiposity and the human gut microbiota (Turnbaugh et al. 2009; Dethlefsen and Relman 2011; Santacruz et al. 2009), plasma metabolome (Cirulli et al. 2019), and host epigenome (Wahl et al. 2017) and genome (Farooqi and O'Rahilly 2007). Genetic and epigenetic modifications that result in transcriptome, proteome, and metabolome changes may have a role in the pathophysiology of energy imbalance seen in obesity.

### Objective

The purpose of this study was to uncover the distinctive markers of abnormal satiation using a multi-omic profile of satiation in patients with obesity.

### Materials and Methods

### Study Population

The study included baseline characteristics from participants recruited between 2010 and 2021. Participants were recruited at Mayo Clinic in Rochester, MN. Ethical approval for the study was obtained from the Mayo Clinic Institutional Review Board (IRB #: 10-007083, 17-003449, and 19-007485) and registered on ClinicalTrials.gov (NCT00433641, NCT003374956, and NCT04073394). Characteristics measured at baseline from all trials were recorded. Eligible participants were patients with obesity (BMI>30 kg/m²); otherwise, healthy with no unstable psychiatric disease, aged 18-75 years. Exclusion criteria included abdominal surgery (other than an appendectomy, laparoscopic cholecystectomy, caesarian section, or tubal ligation); positive history of chronic gastrointestinal diseases or systemic disease that could affect gastrointestinal motility; use of medications that may alter gastrointestinal motility, appetite, or absorption; significant psychiatric dysfunction based upon Hospital Anxiety and Depression Scale (HADS), positive alcoholism screening test (AUDIT-C) and the Questionnaire on Eating and Weight Patterns (binge eating disorders and bulimia). All participants provided written informed consent for study participation and further use of their data, including genetic information.

The validation cohort included participants from a double-blind, placebo-controlled, parallel-group trial testing liraglutide subcutaneous (SQ) 3 mg or placebo for a total treatment period of 16 weeks. Protocol and results have been published elsewhere (Maselli et al. 2022, in press).

### In-Person Physiologic Testing Visit

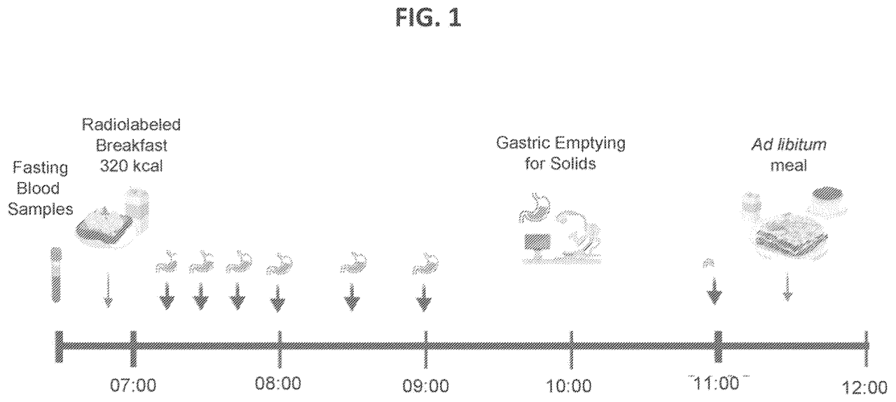

All participants were screened to complete inclusion and exclusion criteria and were e-mailed a pre-visit study pack with questionnaires (i.e., HADS, AUDIT-C, and the Questionnaire on Eating and Weight Patterns). Questionnaires were completed at home and returned to the study staff at the phenotype visit. Patients were asked to report with prior 8-hour fasting and abstain from caffeine the day prior. Participants arrived between 6:00- and 8:00 am at the Clinical Research Trials Unit (CRTU). Heart rate and blood pressure were measured using an automated blood-pressure monitor while participants fasted. Participant weight, height, and hip and waist circumference were measured using standard clinical techniques. Following the anthropometric measurements, participants had a baseline fasting blood draw, consumed a standardized radiolabel breakfast for gastric emptying, and 4 hours later had an ad libitum meal for lunch (FIG. 1). Table 1 shows the baseline discovery cohort characteristics measured in the in-person physiologic testing.

TABLE 1
Baseline discovery cohort characteristics measured in the in-person physiologic testing day.

| | Discovery Cohort | Females | Males | |
|---|---|---|---|---|
| | (n = 717) | (n = 538) | (n = 179) | p-value |
| Age, years | 41.11 ± 11.37 | 41.36 ± 11.01 | 40.34 ± 12.39 | 0.32 |
| Weight, kg | 105.56 ± 22.85 | 102.42 ± 20.74 | 115.01 ± 26.13 | <.0001 |
| Height, cm | 168.91 ± 8.80 | 165.61 ± 6.55 | 178.85 ± 7.04 | <.0001 |
| Harris Benedict, | 1877.61 ± 348.18 | 1747.39 ± 210.61 | 2269.01 ± 385.38 | <.0001 |
| kcal/day | | | | |
| BMI, kg/m² | 36.95 ± 7.13 | 37.30 ± 7.01 | 35.91 ± 7.40 | 0.02 |
| Systolic pulse, | 130.28 ± 14.78 | 128.36 ± 14.55 | 136.06 ± 13.99 | <.0001 |
| mmHg | | | | |
| Diastolic pulse, | 79.13 ± 10.84 | 78.45 ± 10.86 | 81.18 ± 10.53 | 0.003 |
| mmHg | | | | |
| Pulse, bpm | 73.18 ± 11.73 | 74.05 ± 11.32 | 70.56 ± 12.55 | 0.001 |
| Waist circum- | 111.17 ± 15.17 | 110.00 ± 14.57 | 114.66 ± 16.39 | 0.001 |
| ference, cm | | | | |
| Hip circum- | 122.91 ± 13.26 | 124.34 ± 13.27 | 118.63 ± 12.29 | <.0001 |
| ference, cm | | | | |
| Waist/hip, ratio | 0.90 ± 0.07 | 0.88 ± 0.07 | 0.96 ± 0.06 | <.0001 |
| GE 120 min, ratio | 0.53 ± 0.16 | 0.50 ± 0.15 | 0.63 ± 0.16 | <.0001 |
| GE T ½, min | 126.99 ± 32.69 | 132.71 ± 31.48 | 109.88 ± 30.27 | <.0001 |
| Calories to | 917.36 ± 315.40 | 835.24 ± 259.11 | 1164.19 ± 340.52 | <.0001 |
| fullness, kcal | | | | |
| Vas-hunger, mm | 59.93 ± 23.70 | 58.46 ± 24.35 | 65.04 ± 20.63 | 0.0091 |
| VAS-Fullnes, mm | 22.75 ± 19.52 | 23.36 ± 19.84 | 20.66 ± 18.33 | 0.21 |
| VAS-satis- | 26.12 ± 18.45 | 27.14 ± 18.96 | 22.56 ± 16.16 | 0.02 |
| faction, mm | | | | |
| Total Lean | 54744.05 ± 10684.23 | 50542.68 ± 6494.61 | 68935.35 ± 9824.35 | <.0001 |
| Mass, g | | | | |
| Lean Mass, % | 53.09 ± 6.20 | 51.13 ± 4.7377792797 | 59.70 ± 6.03 | <.0001 |
| Fat Mass, % | 48.0 ± 6.0 | 50.16 ± 0.0470912085 | 41.46 ± 6.05 | <.0001 |

Data are presented as mean ± standard deviation
Abbreviations:
BMI, body mass index;
bpm, beats per minute;
GE, gastric emptying

### Outcome Variables and Sample Collection, Handling, and Analysis

### Gastric Emptying for Solids

A standard breakfast of 320 kcal and 30% fat consisting of two 99mTc-radiolabeled eggs, toast, and 80 mL of skim milk was given to participants. Images were obtained immediately after radiolabeled meal ingestion and ever, 30 minutes for the first 2 hours, then every hour for the next 2 hours (total 4 hours after the radiolabeled meal) (Camilleri et al. 2012). Gastric emptying (GE) is summarized by the half-emptying time (GE T_{1/2}) in minutes.

### Appetite Sensations

The visual analog scales (VAS) was measured using a validated, standard, 100 mm appetite VAS for hunger, fullness, desire to eat, and satisfaction score (Flint et al. 2,000; Calderon et al. 2020). Appetite VAS was assessed 15 minutes before breakfast and, then every 30 minutes for the first 2 hours.

### Satiation Test: Ad Libitum Meal

The ad libitum meal included: vegetable lasagna [Stouffers®, Nestle USA, Inc, Solon, OH; nutritional analysis of each 326 g box: 420 kcal, 17 g protein (16% of energy), 38 g carbohydrate (37% of energy), and 22 g fat (47% of energy)]; vanilla pudding [Hunts®, Kraft Foods North America, Tarrytown, NY; nutritional analysis of each 99 g carton: 130 kcal, lg protein (3% of energy), 21 g carbohydrate (65% of energy), and 4.5 g fat (32% of energy)]; and skim milk [nutritional analysis of each 236 mL carton: 90 kcal, 8 g protein (36% of energy), 13 g carbohydrate (64% of energy), and 0 g fat]. The total amount (g and kcal) of food consumed and the kcal of each macronutrient at the ad libitum meal were analyzed by a registered dietitian using validated software (ProNutra 3.0; Viocare Technologies Inc, Princeton, NJ).

Participants were served the prepared meal. Once the subject had partially consumed the meal, they were inquired if additional lasagna, pudding, or milk as desired. Subjects were asked to use any food products until they achieved maximum fullness. As the subject completed each meal component, the empty plate, empty pudding container, and/or liquid volume of milk were weighed back. Once the subject reached maximum fullness, the total weight of food products consumed was summarized. The primary measurement was total kcal consumed to fullness (CTF).

TABLE 2
Baseline characteristics of participants
with normal and abnormal satiation.

| | Abnormal | Normal | |
|---|---|---|---|
| | Satiation | Satiation | p- |
| | (n = 179) | (n = 161) | value |
| Age, years | 39.06 ± 10.63 | 43.18 ± 11.9 | 0.001 |
| Height, m | 1.71 ± 0.09 | 1.68 ± 0.09 | 0.01 |
| Weight, kg | 109.57 ± 22,98 | 104.52 ± 22.67 | 0.04 |
| Harris Benedict, kcal/day | 1938.64 ± 355.28 | 1855.21 ± 359.76 | 0.03 |
| BMI, kg/m2 | 37.64 ± 7.37 | 36.87 ± 6.66 | 0.30 |
| Systolic pulse, mmHg | 129.55 ± 14.76 | 132.48 ± 15.85 | 0.08 |
| Diastolic pulse, mmHg | 76.86 ± 10.83 | 81.3 ± 10.81 | <0.001 |
| Pulse, bpm | 72.24 ± 11.32 | 75.34 ± 12.28 | 0.01 |
| Waist circumference, cm | 112.97 ± 15.72 | 112.74 ± 15.13 | 0.89 |
| Hip circumference, cm | 124.59 ± 13.32 | 122.96 ± 13.11 | 0.26 |
| Waist/hip, ratio | 0.91 ± 0.08 | 0.92 ± 0.08 | 0.24 |
| Gastric emptying | 0.56 ± 0.16 | 0.5 ± 0.16 | <0.001 |
| 120 min, ratio | | | |
| GE T 1/2, min | 111.67 ± 29.79 | 124.57 ± 32.7 | <0.001 |
| Calories to fullness, | 1293.93 ± 260.04 | 618.11 ± 116.8 | <0.001 |
| kcal | | | |

Data are presented as mean ± standard deviation
Abbreviations: BMI, body mass index; bpm, beats per minute; GE, gastric emptying

### Sample Collection and DNA Extraction

Baseline blood samples following an overnight fast were collected from each subject into 6 ml EDTA blood collection tubes. Serum was kept at room temperature and immediately prepared for subsequent analysis of routine laboratory analyses or aliquoted and stored at −80° C. until further analyses. Plasma was obtained from EDTA tubes, immediately placed on ice, and centrifuged within 10 min (1500 g, 4° C., 10 min). Plasma samples were aliquoted and stored at −80° C. until further analyses.

### Metabolomics

The Mayo Clinic Metabolomics Core conducted a quantitative analysis of amino acids and amino acid metabolites in plasma samples using a liquid chromatography-tandem mass spectrometry (LC-MS/MS) method with Waters MassTrack Amino Acid Analysis Solution (Waters, Milford, MA). (Lanza et al. 2010) Plasma samples and amino acid reference standards were prepared using Waters' MassTrak Amino Acid (AA) Analysis Solution kit, with minor modifications for detection on a mass spectrometer. A 10-point standard concentration curve was made from the calibration standard solution to calculate amino acid concentrations in plasma samples. A total of 46 amino acids and metabolites were able to be measured. Table 3 lists metabolites measured in this study.

TABLE 3
List of metabolites measured in this study.

Alanine
Allo-isoleucine
Alpha-amino-n-butyric-acid
Alpha-aminoadipic-acid
Arginine
Asparagine
Asparticacid
Beta.alanine
Beta-amino-iso-butyric acid
Citrulline
Cystine
Ethanolamine
Glutamicacid
Glutamine
Glycine
Histidine
Hydroxyproline
Isolcucine
Leucine
Lysine
Methionine
Methylhistidine-1
Methylhistidine-3
Ornithine
Phenylalanine
Phosphoethanolamine
Proline
Sarcosme
Serine
Taurine
Threonine
Tryptophan
Tyrosine
Valine

### Genotyping

According to the manufacturer's instructions. DNA was extracted from whole blood as previously described (Acosta. Camilleri, Shin, Vazquez-Roque, et al. 2015) by TaqMan® SNP Genotyping Assays (Applied Biosystems, Foster City, CA) using 10-20 ng DNA. Following PCR amplification, end reactions were read on the ABI 7500 Fast Real-Time PCR system using Sequence Detection Software version 1.3.1 (Applied Biosystems). SNPs previously reported to be associated with variation of BMI, postprandial satiation, and with pharmacological response to sibutramine or involved in the control of gastrointestinal functions in a preliminary study were extracted from the full set of genome-wide genotypes using PLINK and were tested for association with gastric emptying using linear regression methods. Selected SNPs are described in Table 4.

TABLE 4
Minor allele frequencies of selected genes and reference for selection in this study.

| | | Minor | | | | | | | |
|---|---|---|---|---|---|---|---|---|---|
| SNP | GENE | Allele | AA | Aa | aa | f(p) | f(q) | MAF | Reference |
| rs7903146 | TCF7L2 | T | 127 | 94 | 19 | 0.73 | 0.28 | 0.28 | (Wrzosek et al. |
| | | | | | | | | | 2019) |
| rs17782313 | MC4R | C | 130 | 87 | 16 | 0.74 | 0.26 | 0.26 | (Yu et al. 2020) |
| rs5443 | GNB3 | T | 103 | 104 | 26 | 0.67 | 0.33 | 0.33 | (Mărginean et al. |
| | | | | | | | | | 2019) |
| rs1129649 | GNB3 | C | 95 | 109 | 29 | 0.64 | 0.36 | 0.35 | (Papathanasopoulos |
| | | | | | | | | | et al. 2010) |
| rs1047776 | GNB3 | A | 174 | 53 | 6 | 0.86 | 0.14 | 0.16 | (Papathanasopoulos |
| | | | | | | | | | et al. 2010) |
| rs659366 | UCP2 | T | 83 | 118 | 32 | 0.61 | 0.39 | 0.37 | (Galmés, Palou, |
| | | | | | | | | | and Serra 2020) |
| rs2075577 | UCP3 | A | 44 | 123 | 66 | 0.45 | 0.55 | 0.53 | (Acosta, Camilleri, |
| | | | | | | | | | Shin, Vazquez- |
| | | | | | | | | | Roque, et al. 2015; |
| | | | | | | | | | van Abeelen et al. |
| | | | | | | | | | 2008) |
| rs15763 | UCP3 | A | 125 | 94 | 14 | 0.74 | 0.26 | 0.25 | (Salopuro et al. |
| | | | | | | | | | 2009) |
| rs1626521 | UCP3 | A | 54 | 67 | 33 | 0.57 | 0.43 | 0.46 | (Acosta, Camilleri, |
| | | | | | | | | | Shin, Vazquez- |
| | | | | | | | | | Roque, et al. 2015) |
| rs3813929 | HTR2C | T | 170 | 54 | 9 | 0.85 | 0.15 | 0.20 | (Miranda et al. |
| | | | | | | | | | 2015) |
| rs11554825 | GPBAR1 | T | 49 | 101 | 83 | 0.43 | 0.57 | 0.60 | (Wrzosek et al. |
| | | | | | | | | | 2019) |

### Predictive Model Creation and Validation

The abnormal satiation phenotype was then determined for patients by creating a machine-learning (ML) model for appetite using the full phenotype tests (FPTs). To generate the ML predictive model, two independent cohorts of patients with obesity were included who each completed the following validated tests at baseline: a) satiation, studied by ad libitum buffet meal (kcal consumed to maximal fullness), and b) gastric emptying of solids studied by scintigraphy. A fasting blood sample was collected for satiety (PYY, GLP1) hormones (ELISA), metabolomics (Mass Spectometry), and DNA (SNP Array). In the first cohort of a total of 799 patients (mean BMI of 38.5±4.38 kg/m²), a multivariate logistic regression ML-driven model for appetite was developed by using the FPTs with all baseline data collected. The linear predictor was calculated with the model's parameter estimates to fit y=a+bx. The ML. FPT model was then validated in an independent cohort of 141 patients (BMI: 39.09±7.40 kg/m²).

### Statistical Analysis

Categorical variables are presented as percentages, and all continuous data are summarized as mean and standard deviation (SD). Descriptive statistics were used to show demographic, anthropometric, and physiologic parameters. Statistical testing of distributions (Abnormal postprandial satiety vs. normal postprandial satiety) were assessed by a two sampled unpaired t-test if data followed a Gaussian distribution or Mann Whitney test if non-Gaussian distribution. All tests were two-tailed, and a p-value <0.05 was considered statistically significant. Multivariable regressions were applied to investigate the association between satiation as continues variable and our exposures (demographics, anthropometrics, vital signs, questionnaires, metabolites, and hormones), and R² values were reported. Statistical analysis was performed using JMP®, Version 14.3.0 (SAS Institute Inc., Cary, NC, 1989-2019) and visualized with GraphPad Prism (Version 8).

### Results

### Cohort Measurements and Heterogeneity of Satiation

To evaluate food intake, and particularly satiation, adults were recruited with obesity (body mass index ≥30 mg/kg²) aged between 21 to 70 years to complete a full day of in-person physiologic testing (FIG. 1). 717 subjects were studied during a full day of in-person physiologic testing. Participants were mostly middle-aged females (75%) and White Americans, with a mean BMI of 36.2 kg/m² (Table 1). The findings were validated with baseline physiologic characteristics of 163 adults with obesity recruited for a weight loss trial, methods and results of the trial have been previously published (Maselli et al. 2022, in press).

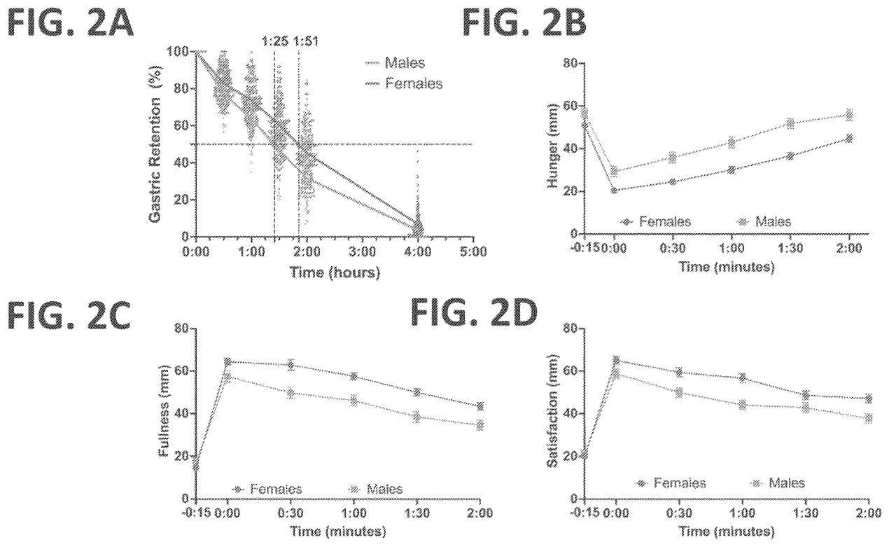

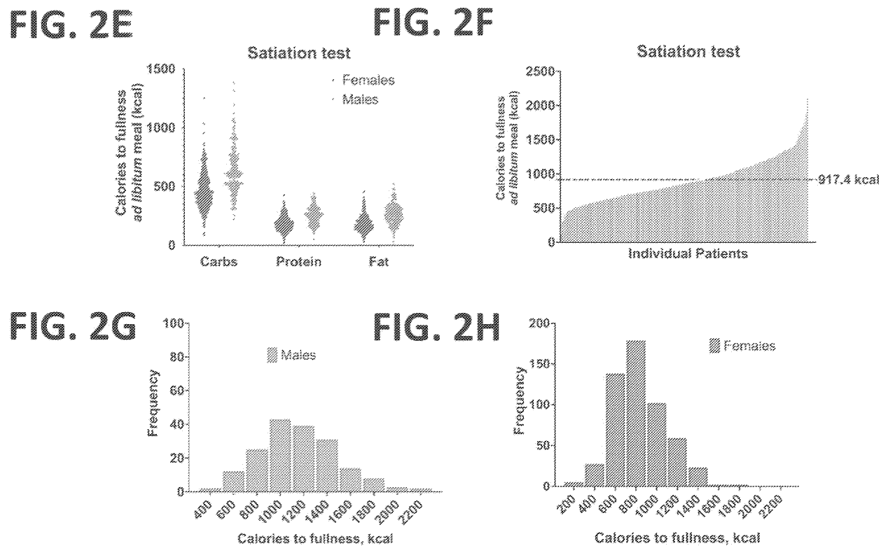

All participants reported after an 8-hour fasting and had a standardized breakfast of 320 kcal with 30% to measure gastric emptying for 4 hours and complete appetite ratings in this period. The mean GE T % was 127.0±32.7 min, females have a slower gastric emptying when compared to males (132.7±31.5 min vs. 109.6±30.3 min; p<0.001) (FIG. 2A). With the same standardized meal, females reported lower levels of hunger and higher levels of fullness and satisfaction than males (FIGS. 2B-D). Following their last scan, participants had their satiation test which consisted of an ad libitum meal. Participants were served the prepared meal and were asked to eat until reaching fullness. The mean calories to fullness were 917.4±315.5 kcal, and females consume fewer calories to reach fullness (835.2±259.1 kcal vs. 1164.2 f 340.5 kcal; p<0.00) (FIG. 2E-H). These highlights the huge heterogeneity in food intake in patients with obesity and the gender differences which required to assess these components separately between females and males.

Predicting Individual Satiation within Patients with Obesity.

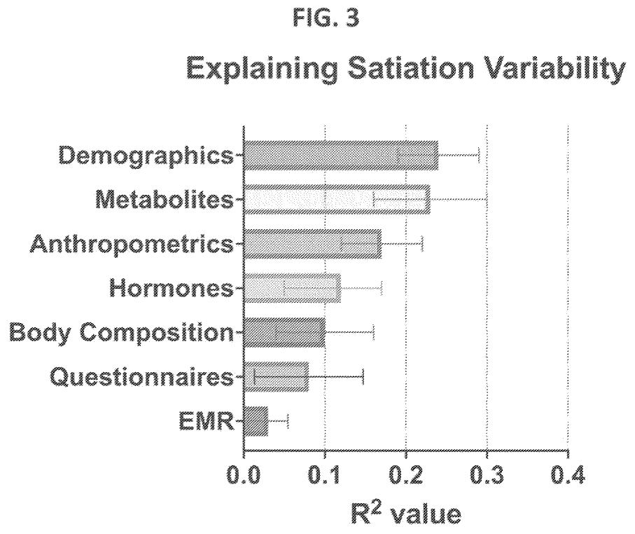

Participants were asked to complete behavioral questionnaires and were assessed by a physician to confirm their medical history. A targeted metabolite panel (Table 3) was measured along with enteroendocrine hormones (i.e., glucagon like peptide 1 [GLP-1], peptide YY [PYY], and ghrelin). All data at different -omics level was used to explain the variability of satiation. The data showed that among the input variables baseline characteristics (age, gender, and race) can explain the most significant proportion of postprandial satiety variance (r²=0.24) (FIG. 3).

### Characterization of Abnormal Satiation

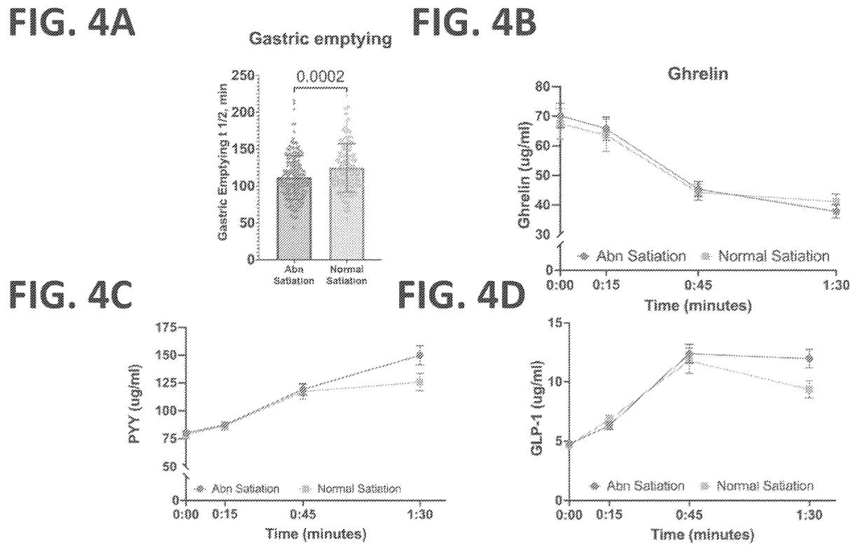

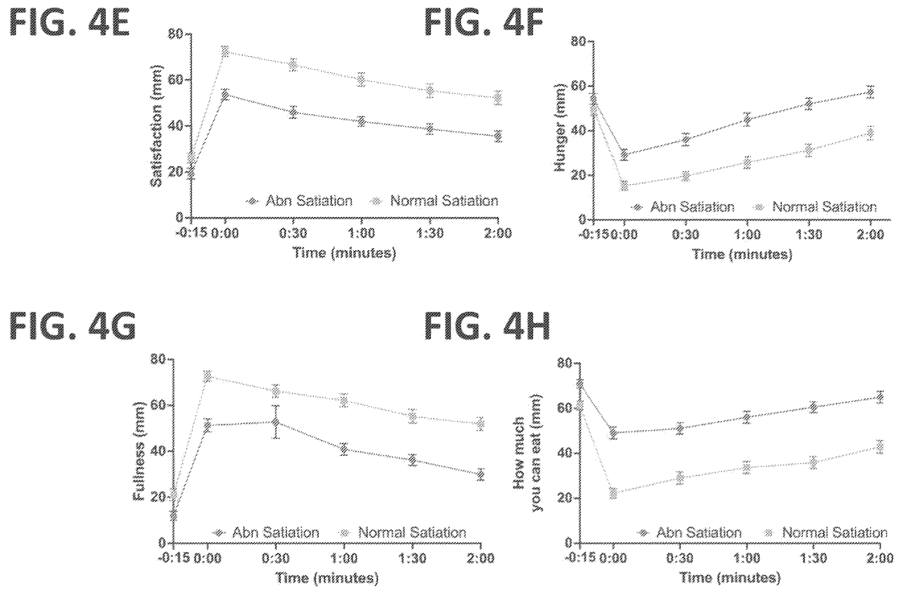

Next, the cohort was divided into quartiles by gender and participants with normal and abnormal satiation were compared. In the normal satiation group, females consumed less than 650 kcal to reach fullness and males less than 927 kcal. While in the abnormal satiation group, females consumed more than 977 kcal to reach fullness and males more than 1374 kcal (Table 2). First, gastric emptying between groups was evaluated, participants with abnormal satiation have a faster gastric emptying with 111.7±29.8 min half emptying time compared with 124.5±32.7 min in the normal satiation group (FIG. 4A). The hormonal curves between groups was then evaluated. There was no difference in ghrelin, but participants with abnormal satiation were observed have higher levels of GLP-1 (11.98±8.5 ug/ml vs. 9.36±6.9 ug/ml) and PYY (11.98±8.5 ug/ml vs. 9.36±6.9 ug/ml) after 90 minutes of a standardized meal of 320 kcal (30% fat) (FIGS. 4B-D). Finally, appetite sensations between groups were compared, and participants with abnormal satiation had more hunger and less satisfaction at all timepoints after their breakfast, lower fullness at baseline and at 60, 90, and 120 minutes (FIGS. 4E-G). And in general, participants with abnormal satiation wanted to eat more than their control group at all timepoints. There are large interpersonal differences in satiation, but this classification allowed characterization of this group of participants who present a different subjective response measured with appetite scales and objective response measured with gastric emptying and postprandial hormonal levels to the same meal.

### Prediction and Validation of Normal and Abnormal Satiation

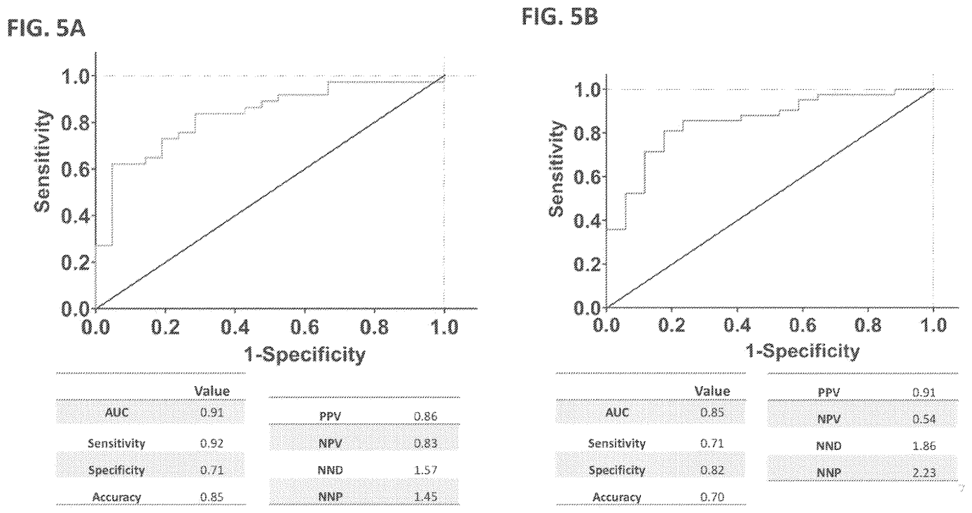

Next, clinical and -omics factors were integrated into an algorithm that differentiate normal vs. abnormal satiation (i.e., the machine-learning (ML) model for appetite using the full phenotype tests (FPTs). To this end, the algorithm was developed on the main cohort of 799 participants, and performance was evaluated in the validation cohort of 141 subjects that completed the same assessments. Integrating demographics, anthropometrics, and fasting blood parameters, the ML-driven, full phenotype biomarker had an AUC of >0.80, while the validation cohort had an AUC of 0.84 (FIG. 5).

### Validation of Normal and Abnormal Satiation Discrimination for Prediction of Response to Liraglutide

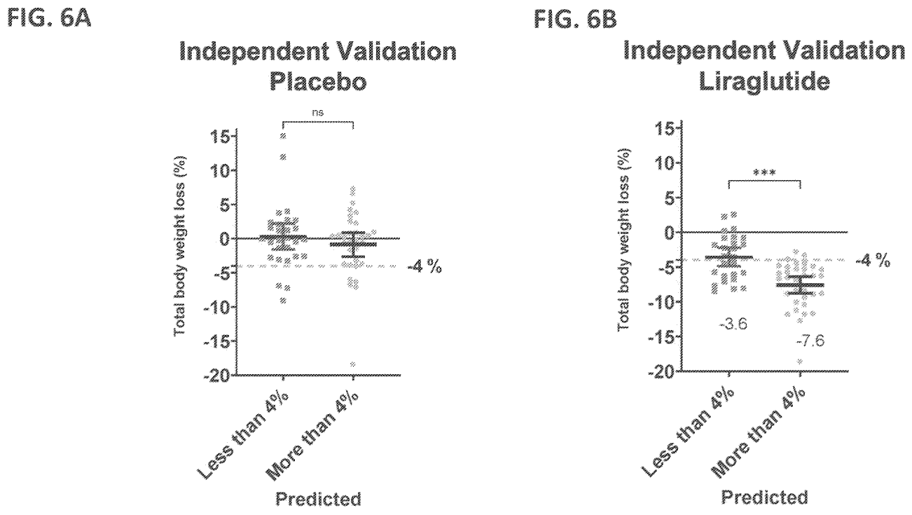

To assess the clinical relevance of the predictor model, we tested the predictions with short term response to liraglutide were tested using the ML-driven, full phenotype biomarker. Previously, baseline characteristics have not been able to discriminate responders from non-responders. Energy intake has been used as a clinical measure to assess the effect of liraglutide on weight loss(Van Can et al. 2014; Horowitz et al. 2012; Flint, Kapitza, and Zdravkovic 2013). And weight loss was found to be associated with a decrease in kcal consumed during ad libitum meal in two studies(Van Can et al. 2014; Flint, Kapitza, and Zdravkovic 2013). Responders were classified based on a clinically significant response of greater than 5% total body weight lost and the ML-driven, full phenotype biomarker model was used to discriminate responders and non-responders. In particular, the performance of the FPT (n=60) was tested in predicting response to treatment with liraglutide (LIRA) 3 mg SQ/day from a randomized, placebo-controlled trial of 16 weeks. A response was defined as >5% total body weight loss [TBWL %]. Analysis was done using student's T-test to compare among the predicted groups. In participants with liraglutide, the ML-driven, full phenotype biomarker model had an AUC of 0.70 to identify responders (FIG. 6A). To confirm its utility to detect response to liraglutide rather than just weight loss, the model was tested inpatients with placebo and the model had an AUC of 0.63 (CI 0.53 to 0.73) (FIG. 6B). Finally, the actual total body weight loss percentage was evaluated in participants predicted as responders (i.e., patients with a non-hungry brain or non-abnormal satiation phenotype) and non-responders (i.e., patients with a hungry brain or abnormal satiation phenotype) and the TBWL % in groups predicted as responder vs. non-responder using FPT was −7.6±3.4 vs. −3.6±3.3; p<0.001 (FIG. 6C). The same testing was performed in the placebo group, and here, the difference was non-significant between groups (FIG. 6D).

### DISCUSSION

Individual aspects of satiation were explored at different omic levels and a prediction model for abnormal satiation was developed. Basic variables, such as demographics or anthropometrics, cannot explain observed variability and are insufficient in predicting abnormal postprandial satiety, highlighting the importance of deep phenotyping incorporating many variables in characterizing physiologic components of food intake. The ML, FPT prediction model developed in this Example had an AUC>0.80 and was validated in an independent cohort of patients with obesity. And this allowed discrimination of responders to GLP-1 analogues in a short-term placebo-controlled trial.

Individual variability in predisposition to weight gain and resistance to weight reduction may partly be explained by inter-individual variability in all stages of food intake, hunger, satiation, and satiety. (Gibbons et al. 2019) Abnormal satiation is thought to be a risk factor for overconsumption (Drapeau et al. 2007) and is associated with obesity. (Acosta. Camilleri, Shin, Vazquez-Roque, et al. 2015).

### Example 2—Development of Obesity Phenotype Polygenic Risk Score/Machine Learning Predictor

### Background

Human genetics drive our propensity to have certain characteristics. These characteristics can often be complex and result from differences in several parts of our genome. Now that technologies are available to identify genetic differences between individuals (variants), the challenge becomes identifying which variants are relevant to a particular characteristic, such as susceptibility to a disease. Being able to identify relevant variants and how they contribute to a characteristic enables prediction of, among other things, disease risk. One method known in that art that can do this identification and can predict the likelihood of a characteristic are Polygenic Risk Scores (PRSs).

A PRS typically starts with a basic regression or statistical model to identify the relevance of a SNP. Using a regression model, the relevance is the effect size of a SNP (i.e., beta value of the regression). Using a statistical model, the relevance is whether the p-value of association between the SNP and the particular trait is less that a certain threshold. Whichever the approach, the information about the variants in a particular sample are combined. For regression models, it may be the sum of the beta values for SNPs with an effect allele, while for statistical models, the count of the number of SNPs below the p-value threshold with an effect allele. Additional transformations can be involved, such as various forms of normalization, as well as numerous quality control steps, such as accounting for linkage disequilibrium. However, these approaches have several shortcomings.

One basic shortcoming is that identifying which variants are relevant can be ambiguous due to the structure of genetic inheritance. Further, the low statistical power of the basic genetic analyses underlying PRSs can create PRSs that are irrelevant to the characteristic. Because the number of samples required to have sufficient statistical power to overcome these sources of ambiguity is infeasible, a different approach was warranted.

### Objective

The purpose of this study was to develop a new type of PRS that integrates expert guidance, machine learning, and non-genetic/multi-omic data that addresses the shortcomings of standard genetic analysis and can serve as a simpler, DNA only, surrogate to the ML, FTP obesity phenotype predictor developed in Example 1. The approach taken in this Example side stepped resolving ambiguity by allowing it to be present in initial steps of generating a PRS and in later steps used expert and data-derived guidance to minimize ambiguity. Further, while the approach used herein uses some standard PRS components, such as quality control steps to control from sample relatedness and regression between genetics of a set of samples and a particular trait, the approach used herein avoided the irrelevance of naively scoring a particular characteristic directly and instead sought to first estimate the contribution of an individual's genetics to a set of traits associated with the particular characteristic.

### Materials and Methods

Conceptually, the adaptive contribution-factor-based PRS of this Example was developed as follows:

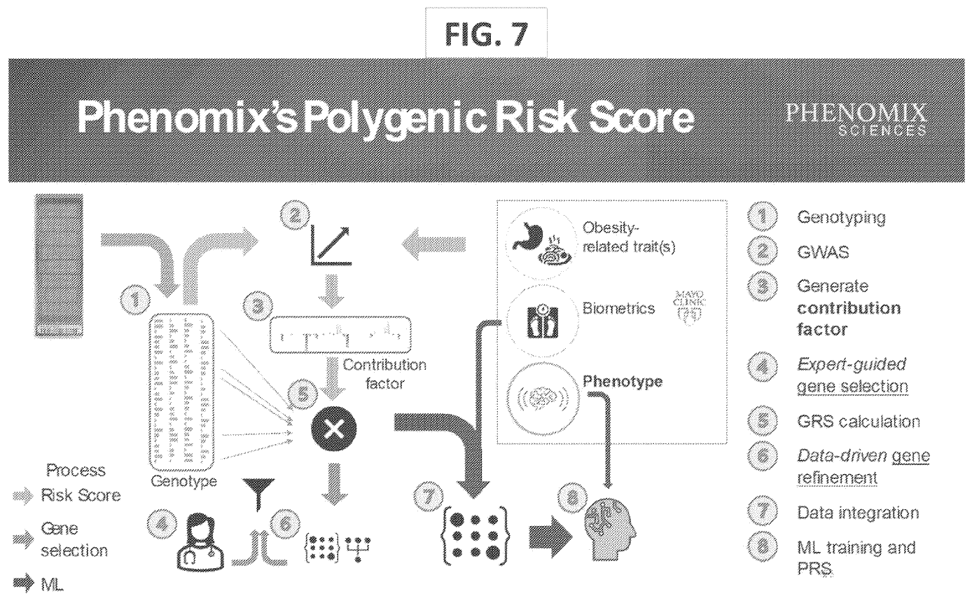

Rather than using regression beta values across the genome naively, the beta in the regression model was transformed using an algorithm that encoded biological factors about genetics, epigenetics, gene regulation, and similar, for each variant. The output numerical value representing the contribution of a variant to a trait after this algorithm runs was termed the Contribution Factor. This was step (3) in the diagram shown in FIG. 7.

Rather than using SNPs independently, the effect variants at SNPs may have on a particular gene were summarized, creating an intermediary Gene Risk Score (GRS). To generate a GRS, the dosage of variants around a gene were integrated with the contribution factor of each SNP. The resulting value was a risk score that represented the likelihood that the variants around that gene change the function or regulation of that gene, which in turn plays a role in the characteristic.

Because the gene risk scores reflected a particular trait, and not the characteristic to be predicted, extra steps were taken to identify gene risk scores that best explained the target characteristic. Expert guidance and machine learning techniques were combined to iteratively remove irrelevant genes (rather than variants individually). This iterative process ended when the gene list was whittled down to the shortest list of genes whose gene risk scores for a set of samples best explained the characteristic in those samples. This was the Adaptive steps (4), (5), and (6) in the diagram shown in FIG. 7.

Finally, the PRS combined these intermediary gene risk scores for all selected traits as well as non-genetic and/or multi-omic data (step (7) in FIG. 7) to predict the target characteristic. This prediction was the adaptive contribution-factor-based polygenic risk score (step (8) in FIG. 7).

In this Example, an adaptive contribution-factor-based PRS was developed to predict the Hungry Brain (i.e., abnormal satiation) obesity phenotype. Hungry Brain is a type of obesity driven by a defect in satiation: the brain does not properly signal that the stomach is full, leading to overconsumption of calories. Hungry Brain is defined as consuming above the 75% ile amount of calories at an ad libitum meal. SNP variant information from quality-controlled samples from 497 of the 799-patient cohort (mean BMI of 38.5±4.38 kg/m²) referenced in Example, was obtained using a SNP chip. Quantitative traits were also collected for these 497 patients. A regression between the variants in these samples and a trait called ‘buffet meal’ was performed. This ‘buffet meal’ trait was the number of calories consumed at an ad libitum meal. Because this trait was substantially different in males vs females, separate regressions for male samples and female samples were performed rather than including them as a cofactor. In particular, a LASSO regression through the PLINK 2.0 tool using default settings was used to perform the regression analyses.

Subsequently, contribution factors and gene risk scores were created, and the adaptive step of the process outlined in FIG. 7 was performed. The contribution factor was derived from the beta value but was the output of a function that also takes other parameters into account. The other parameters included the proximity of the variant to the gene, apparent mechanistic role of the variant (e.g., as within an intron vs exon vs enhancer region), or metadata about the SNP in relationship to the gene, such as the likelihood that it alters expression of the gene. The contribution factor and dosage information of SNPs within each gene and withing 500 KB of that gene were then integrated to create a gene risk score for each gene. Importantly, the GRS was not calculated for all known genes, but only for an expert-guided set of 22 genes. In fact, for each of these genes, two GRSs were created, one using contribution factors based on the male regression model and the other based on the female regression model. Using forward feature selection and random forest predictors, a plurality of GRSs across a plurality of the genes were identified that are predictive of the Hungry Brain phenotype in the training cohort, with the remaining GRSs not providing any further benefit.

Finally, a PRS using the selected GRSs was created and validated in an independent cohort of 141 patients (BMI: 39.09±7.40 kg/m²). The final PRS was built using the 13 selected GRSs normalized with a Yeo power transformation and the height of the individual using a support vector classifier (SVC). The SVC was trained using a linear kernel and a C value of 0.001 using SMOTE oversampling of the 497 samples. The final PRS was then the trained classifier's predicted probability of Hungry Brain (e.g., abnormal satiation).

Subsequently, the performance of the PRS (n=40) model in predicting response to treatment with liraglutide (LIRA) 3 mg SQ/day from a randomized, placebo-controlled trial of 16 weeks was tested. Like in Example 1, a response was defined as >5% total body weight loss [TBWL %]. Analysis was done using student's T-test to compare among the predicted groups.

### Results and Conclusions

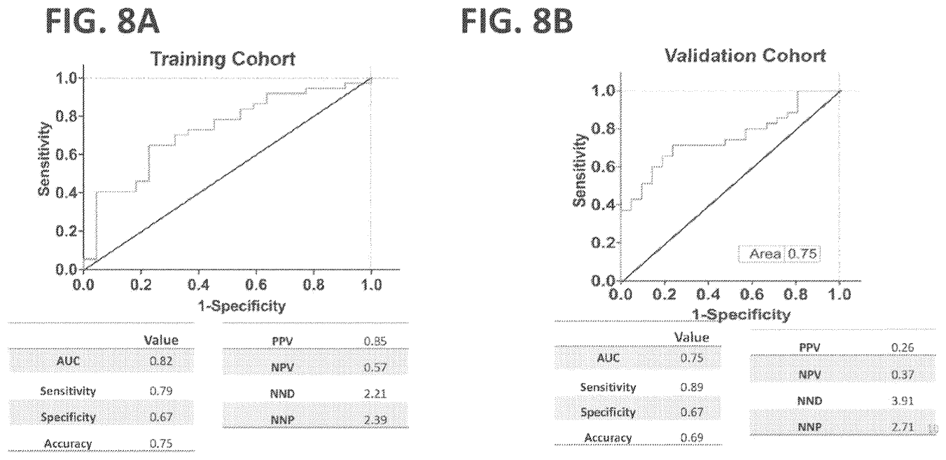

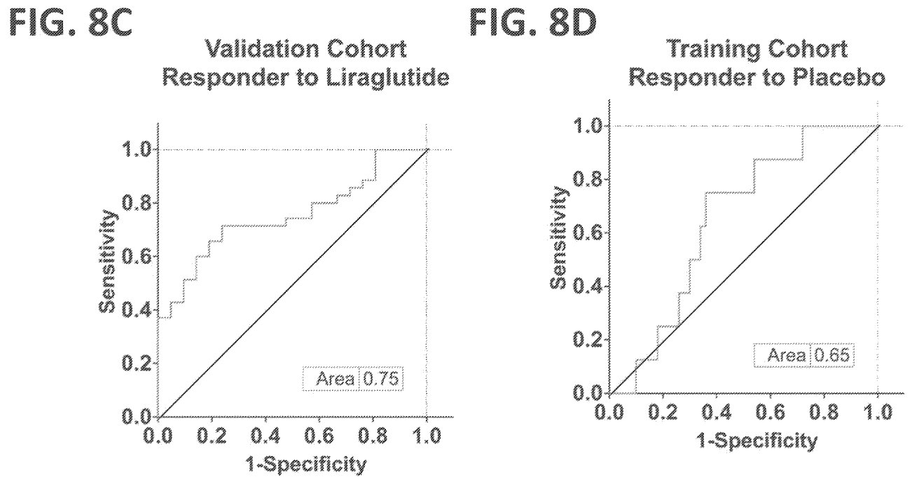

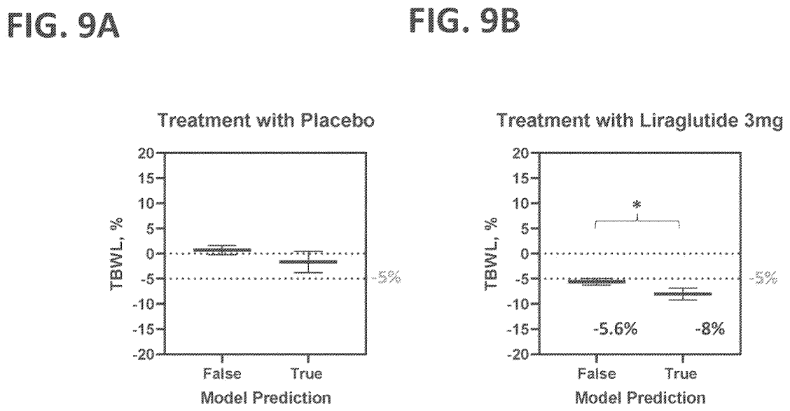

In the training cohort the PRS model had an AUC>0.80. In the independent validation cohort, the PRS had an AUC=0.70 with a sensitivity of 0.71, specificity of 0.65, and PPV of 0.53 was measured (see FIGS. 8A-8B). Note that because the phenotype was defined as being above the 75% ile of calories consumed, a priori a PPV of 0.25 for random guessing. When used to predict response to liraglutide, the PRS an AUC=0.69. In patients treated with liraglutide, the TBWL % in groups predicted as responder vs. non-responder using the PRS was −8.0±4.4 vs. −5.6±3.3, p=0.056 (FIGS. 9A-9B). There was no difference among the predicted groups in patients who received placebo.

The ML-driven, full phenotype biomarker and its PRS surrogate for appetite can predict response to treatment with a GLP-1 analogue. The findings support a novel approach for precision medicine for obesity. The PRS model might be a simpler test to deploy in the clinical practice.

### REFERENCES

Acosta, Andres, Michael Camilleri, Andrea Shin, Maria I. Vazquez-Roque, Johanna Iturrino, Duane Burton, Jessica O'Neill, Deborah Eckert, and Alan R. Zinsmeister. 2015. ‘Quantitative gastrointestinal and psychological traits associated with obesity and response to weight-loss therapy’, Gastroenterology, 148: 537-46.e4.
Acosta, Andres, Michael Camilleri, Andrea Shin, Maria I Vazquez-Roque, Johanna Iturrino, Ian R Lanza, K Sreekumaran Nair, Duane Burton, Jessica O'Neill, and Deborah Eckert. 2015. ‘Association of UCP-3 rs1626521 with obesity and stomach functions in humans’, Obesity, 23: 898-906.
Aleksandrova, K., C. Egea Rodrigues, A. Floegel, and W. Ahrens. 2020. ‘Omics Biomarkers in Obesity: Novel Etiological Insights and Targets for Precision Prevention’, Curr Obes Rep, 9: 219-30.
Aronne. Louis J, Kevin D Hall, John M. Jakicic, Rudolph L Leibel, Michael R Lowe, Michael Rosenbaum, and Samuel Klein. 2021. ‘Describing the weight-reduced state: Physiology, behavior, and interventions’, Obesity. 29: S9-S24.
Blundell, J E, and J C G Halford. 1994. ‘Regulation of nutrient supply: the brain and appetite control’, Proceedings of the Nutrition Society. 53: 407-18.
Calderon, G., A. McRae, J. Rievaj, J. Davis, I. Zandvakili, S. Linker-Nord, D. Burton, G. Roberts, F. Reimann, B. Gedulin, A. Vella, N. F. LaRusso, M. Camilleri, F. M. Gribble, and A. Acosta. 2020. ‘Ileo-colonic delivery of conjugated bile acids improves glucose homeostasis via colonic GLP-1-producing enteroendocrine cells in human obesity and diabetes’, EBioMedicine, 55: 102759.
Camilleri, M., J. Iturrino, A. Bharucha, D. Burton, A. Shin, I. D. Jeong, and A. Zinsmeister. 2012. ‘Performance characteristics of scintigraphic measurement of gastric emptying of solids in healthy participants’, Neurogastroenterology and motility: the official journal of the European Gastrointestinal Motility Society, 24: 1076.
Camilleri, Michael. 2015. ‘Peripheral mechanisms in appetite regulation’, Gastroenterology, 148: 1219-33.
Cannon, Walter B, and Anton L Washburn. 1993. ‘An Explanation of Hunger 1’, Obesity research, 1: 494-500.
Christofferson, Berit Ostergaard, Guillermo Sanchez-Delgado, Linu Mary John, Donna H Ryan, Kirsten Raun, and Eric Ravussin. 2022. ‘Beyond appetite regulation: Targeting energy expenditure, fat oxidation, and lean mass preservation for sustainable weight loss’, Obesity, 30: 841-57.
Choi S W, Mak T S, O'Reilly P F. Tutorial: a guide to performing polygenic risk score analyses. Nat Protoc. 2020 September; 15(9):2759-2772. doi: 10.1038/s41596-020-0353-1. Epub 2020 Jul. 24. PMID: 32709988; PMCID: PMC7612115.
Cifuentes, Lizeth, and Andres Acosta. 2022. ‘Homeostatic regulation of food intake’. Clinics and Research in Hepatology and Gastroenterology, 46: 101794.
Cirulli, E. T., L. Guo, C. Leon Swisher, N. Shah, L. Huang, L. A. Napier, E. F. Kirkness, T. D. Spector, C. T. Caskey, B. Thorens, J. C. Venter, and A. Telenti. 2019. ‘Profound Perturbation of the Metabolome in Obesity Is Associated with Health Risk’, Cell Metab, 29: 488-500.e2.
Collaborators, G. B. D. Obesity, A. Afshin, M. H. Forouzanfar, M. B. Reitsma, P. Sur, K. Estep, A. Lee, L. Marczak, A. H. Mokdad, M. Moradi-Lakeh, M. Naghavi, J. S. Salama, T. Vos, K. H. Abate, C. Abbafati, M. B. Ahmed, Z. Al-Aly, A. Alkerwi, R. Al-Raddadi, A. T. Amare, A. Amberbir, A. K. Amegah, E. Amini, S. M. Amrock, R. M. Anjana, J. Amlov, H. Asayesh, A. Banerjee, A. Barac, E. Baye, D. A. Bennett, A. S. Beyene, S. Biadgilign, S. Biryukov, E. Bjertness, D. J. Boneya, I. Campos-Nonato, J. J. Carrero, P. Cecilio, K. Cercy, L. G. Ciobanu, L. Cornaby, S. A. Damtew, L. Dandona, R. Dandona, S. D. Dharmaratne, B. B. Duncan, B. Eshrati, A. Esteghamati, V. L. Feigin, J. C. Femandes, T. Furst, T. T. Gebrehiwot, A. Gold, P. N. Gona, A. Goto, T. D. Habtewold, K. T. Hadush, N. Hafezi-Nejad, S. I. Hay, M. Horino, F. Islami, R. Kamal, A. Kasaeian, S. V. Katikireddi, A. P. Kengne, C. N. Kesavachandran, Y. S. Khader, Y. H. Khang, J. Khubchandani, D. Kim, Y. J. Kim, Y. Kinfu, S. Kosen, T. Ku, B. K. Defo, G. A. Kumar. H. J. Larson, M. Leinsalu, X. Liang, S. S. Lim, P. Liu, A. D. Lopez, R. Lozano, A. Majeed, R. Malekzadeh, D. C. Malta, M. Mazidi, C. McAlinden, S. T. McGarvey, D. T. Mengistu, G. A. Mensah, G. B. M. Mensink, H. B. Mezgebe, E. M. Mirrakhimov, U. O. Mueller, J. J. Noubiap, C. M. Obermeyer, F. A. Ogbo, M. O. Owolabi, G. C. Patton, F. Pourmalek, M. Qorbani, A. Rafay, R. K. Rai, C. L. Ranabhat, N. Reinig, S. Safiri, J. A. Salomon, J. R. Sanabria, I. S. Santos, B. Sartorius, M. Sawhney, J. Schmidhuber, A. E. Schutte, M. I. Schmidt, S. G. Sepanlou, M. Shamsizadeh, S. Sheikhbahaei, M. J. Shin, R. Shiri, I. Shiue, H. S. Roba, D. A. S. Silva, J. I. Silverberg, J. A. Singh, S. Stranges, S. Swaminathan, R. Tabares-Scisdedos, F. Tadese, B. A. Tedla, B. S. Tegegne, A. S. Terkawi, J. S. Thakur, M. Tonelli, R. Topor-Madry, S. Tyrovolas. K. N. Ukwaja, O. A. Uthman, M. Vaezghasemi, T. Vasankari, V. V. Vlassov, S. E. Vollset, E. Weiderpass, A. Werdecker, J. Wesana, R. Westerman, Y. Yano, N. Yonemoto, G. Yonga, Z. Zaidi, Z. M. Zenebe, B. Zipkin, and C. J. L. Murray. 2017. ‘Health Effects of Overweight and Obesity in 195 Countries over 25 Years’, N Engl J Med, 377: 13-27.
Dethlefsen, Les, and David A Relman. 2011. ‘Incomplete recovery and individualized responses of the human distal gut microbiota to repeated antibiotic perturbation’, Proceedings of the National Academy of Sciences, 108: 4554-61.
Drapeau, Vicky, Neil King, Marion Hetherington, Eric Doucet, John Blundell, and Angelo Tremblay. 2007. ‘Appetite sensations and satiety quotient: predictors of energy intake and weight loss’, Appetite, 48: 159-66.
Farooqi, I. S., and S. O'Rahilly. 2007. ‘Genetic factors in human obesity’, Obes Rev, 8 Suppl 1: 37-40.
Flint, A, C Kapitza, and M Zdravkovic. 2013. ‘The once-daily human GLP-1 analogue liraglutide impacts appetite and energy intake in patients with type 2 diabetes after short-term treatment’, Diabetes, Obesity and Metabolism. 15: 958-62.
Flint, A., A. Raben, J. E. Blundell, and A. Astrup. 2,000. ‘Reproducibility, power and validity of visual analogue scales in assessment of appetite sensations in single test meal studies’, Int J Obes Relat Metab Disord. 24: 38-48.
Galmés, S., A. Palou, and F. Serra. 2020. ‘Increased Risk of High Body Fat and Altered Lipid Metabolism Associated to Suboptimal Consumption of Vitamin A is Modulated by Genetic Variants rs5888 (SCARB1), rs1800629 (UCP1) and rs659366 (UCP2)’, Nutrients, 12.
Gibbons, Catherine, Mark Hopkins, Kristine Beaulieu, Pauline Oustric, and John E Blundell. 2019. ‘Issues in measuring and interpreting human appetite (satiety/satiation) and its contribution to obesity’, Current Obesity Reports, 8: 77-87.
Halawi, H., M. Camilleri, A. Acosta. M. Vazquez-Roque, I. Oduyebo, D. Burton, I. Busciglio, and A. R. Zinsmeister. 2017. ‘Relationship of gastric emptying or accommodation with satiation, satiety, and postprandial symptoms in health’, Am J Physiol Gastrointest Liver Physiol, 313: G442-g47.
Hill, James O. Holly R Wyatt, and John C Peters. 2012. ‘Energy balance and obesity’. Circulation, 126: 126-32.
Horowitz, Michael, Anne Flint, Karen L Jones, Charlotte Hindsberger, Mads F Rasmussen, Christoph Kapitza, Selena Doran, Thomas Jax, Milan Zdravkovic, and Ian M Chapman. 2012. ‘Effect of the once-daily human GLP-1 analogue liraglutide on appetite, energy intake, energy expenditure and gastric emptying in type 2 diabetes’, Diabetes Research and Clinical Practice, 97: 258-66.
Lanza, Ian R, Shucha Zhang, Lawrence E Ward, Helen Karakelides, Daniel Raftery, and K Sreekumaran Nair. 2010. ‘Quantitative metabolomics by 1H-NMR and LC-MS/MS confirms altered metabolic pathways in diabetes’, PLoS One, 5: e10538.
Mǎrginean, C., C. O. Mǎrgincan, C. Bǎnescu, L. E. Melit, F. Tripon, and M. Iancu. 2019. ‘The relationship among GNB3 rs5443, PNPLA3 rs738409, GCKR rs780094 gene polymorphisms, type of maternal gestational weight gain and neonatal outcomes (STROBE-compliant article)’, Medicine (Baltimore), 98: e16414.
Maselli, Daniel, Jessica Atieh, Matthew M. Clark, Deborah Eckert, Ann Taylor, Paula Carlson, Duane D. Burton, Irene Busciglio, W. Scott Harmsen, Adrian Vella, Andres Acosta, and Michael Camilleri. 2022, in press. ‘Effects of liraglutide on gastrointestinal functions and weight in obesity: a randomized clinical and pharmacogenomic trial’. Obesity.
Miranda, R. C., S. B. Vetter, J. P. Genro, P. D. Campagnolo, V. S. Mattevi, M. R. Vitolo, and S. Almeida. 2015. ‘SLC6A14 and 5-HTR2C polymorphisms are associated with food intake and nutritional status in children’, Clin Biochem, 48: 1277-82.
Papathanasopoulos, A., M. Camilleri, P. J. Carlson, A. Vella, S. J. Nord, D. D. Burton, S. T. Odunsi, and A. R. Zinsmeister. 2010. ‘A preliminary candidate genotype-intermediate phenotype study of satiation and gastric motor function in obesity’, Obesity (Silver Spring), 18: 1201-11.
Salopuro, T., L. Pulkkinen, J. Lindström, M. Kolehmainen, A. M. Tolppanen, J. G. Eriksson, T. T. Valle, S. Aunola, P. Ilanne-Parikka, S. Keininen-Kiukaanniemi, J. Tuomilehto, M. Laakso, and M. Uusitupa. 2009. ‘Variation in the UCP2 and UCP3 genes associates with abdominal obesity and serum lipids: the Finnish Diabetes Prevention Study’, BMC Med Genet, 10: 94.
Santacruz, Arlette. Ascensión Marcos, Julia Wärnberg, Amelia Marti, Miguel Martin-Matillas, Cristina Campoy, Luis A Moreno, Oscar Veiga, Carlos Redondo-Figuero, and Jesús M Garagorri. 2009. ‘Interplay between weight loss and gut microbiota composition in overweight adolescents’, Obesity, 17: 1906-15.
Turnbaugh, Peter J. Micah Hamady, Tanya Yatsunenko, Brandi L Cantarel, Alexis Duncan, Ruth E Ley, Mitchell L Sogin, William J Jones, Bruce A Roe, and Jason P Affourtit. 2009. ‘A core gut microbiome in obese and lean twins’, Nature, 457: 480-84.
van Abeelen, A. F., M. de Krom, J. Hendriks, D. E. Grobbee, R. A. Adan, and Y. T. van der Schouw. 2008. ‘Variations in the uncoupling protein-3 gene are associated with specific obesity phenotypes’, Eur J Endocrinol, 158: 669-76.
Van Can, J, B Sloth, C B Jensen, A Flint, E E Blaak, and WHM Saris. 2014. ‘Effects of the once-daily GLP-1 analog liraglutide on gastric emptying, glycemic parameters, appetite and energy metabolism in obese, non-diabetic adults’, International Journal of Obesity, 38: 784-93.
Wahl, Simone, Alexander Drong, Benjamin Lehne, Marie Loh, William R Scott, Sonja Kunze, Pei-Chien Tsai, Janina S Ried, Weihua Zhang, and Youwen Yang. 2017. ‘Epigenome-wide association study of body mass index, and the adverse outcomes of adiposity’, Nature, 541: 81-86.
Wrzosek, M., A. Sawicka, M. Wrzosek, P. Piatkiewicz, M. Talalaj, and G. Nowicka. 2019. ‘Age at onset of obesity, transcription factor 7-like 2 (TCF7L2) rs7903146 polymorphism, adiponectin levels and the risk of type 2 diabetes in obese patients’, Arch Med Sci, 15: 321-29.
Yu, K., L. Li, L. Zhang, L Guo, and C. Wang. 2020. ‘Association between MC4R rs17782313 genotype and obesity: A meta-analysis’, Gene, 733: 144372.

### Example 3—A Polygenic Risk Score to Identify Abnormal Satiation Predicts Response to Phentermine-Topiramate

### Background

Anti-obesity medications have highly variable outcomes. The inventors proposed an obesity classification based on phenotypes to address this heterogeneity in weight loss response. In a 2-week randomized clinical trial (RCT), the inventors showed that one of the phenotypes, abnormal satiation, measured with calories to fullness (CTF) during an ad libitum buffet meal (>75th percentile) had superior weight loss outcomes with phentermine-topiramate ER (phen-top). However, current methods used to identify phenotypes are time-consuming, invasive, expensive, and limited to a few academic centers. Here, a biomarker for abnormal satiation has been developed and validated and its efficacy was assessed to predict weight loss outcomes in a RCT of phen-top vs placebo.

### Methods

The biomarker for abnormal satiation was developed in an independent cohort of 497 patients using a polygenic risk score (PRS) derived from an expert-guided set of SNPs in 22 genes, and demographic characteristics. This model was validated in a new independent cohort of 141 patients. A post-hoc analysis of a 12-month, double-blinded, single-center RCT was conducted that randomized 80 patients with obesity to either placebo (n=38) or phen-top (n=42). These patients completed phenotype testing at baseline. Wilcoxon tests were used to compare the total body weight loss (TBWL) among patients with or without abnormal satiation, defined by either CTF in an ad libitum buffet meal or with the biomarker, in patients assigned to phen-top or placebo.

### Results

In the independent validation cohort, the biomarker for abnormal satiation had a ROC=0.72. In the trial, at 12-months, the phen-top group had a TBWL of 16.4 kg (10.5), as compared to 6 kg (11.8) (p=0.005) in placebo. At 12 months, within patients assigned to phen-top, those with abnormal satiation defined by CTF (40%) had a higher TBWL than the normal satiation group [23.1 kg (11.9) vs. 12.8 kg (8.0), p=0.02]. The biomarker performance for predicting a TBWL>15% at 12 months in patients assigned to phen-top had an ROC=0.88, and those with a positive test (20%) had a higher TBWL than those with a negative test 130 kg (3.9) vs. 12.8 kg (1.9), p=0.0061. There was no difference in the placebo group with either approach.

Conclusion: Abnormal satiation phenotype, measured by CTF in ad libitum buffet meal or by a polygenic risk score, predicts response to phentermine-topiramate ER.

### Example 4—A Machine-Learning-Assisted Gene Risk Score to Identify Abnormal Satiation and it Use in Predicting Response to Anti-Obesity Interventions

### Objective

This example aims to explore the variability of human satiation, a key component of energy intake, and its potential implications for precision obesity management. Through this, satiation was revealed to be highly variable among individuals, and while baseline characteristics, anthropometrics, body composition and hormones, contribute to this variability, they alone cannot account for the totality of the variance in satiation. To address this gap, the role of a polygenic risk score was explored, which demonstrated a robust association with satiation. Furthermore, a machine-learning gene risk score was introduced to predict satiation and to assess this score in the prediction of responses to anti-obesity medications.

### Methods

### Study Population

Baseline characteristics were collected from participants with obesity recruited between 2010 and 2021 at Mayo Clinic, Rochester, MN. Eligible participants were adults aged 18 to 75 years, with obesity (BMI>30 kg/m2), with or without type 2 diabetes, and otherwise healthy. Exclusion criteria included a history of abdominal surgery (except for appendectomy, laparoscopic cholecystectomy, cesarean section, or tubal ligation), chronic gastrointestinal diseases, systemic diseases affecting gastrointestinal motility, and the use of medications that may alter gastrointestinal motility, appetite, or absorption. Ethical approval for the use of data and samples from prior studies for the characterization and development of a biomarker was obtained from the Mayo Clinic Institutional Review Board (IRB #: 17-000838). All participants provided written informed consent for study participation, including the use of their genetic information in future studies.

### Study Cohorts:

This study included two distinct cohorts of participants. The initial study cohort comprised a total of 717 participants who completed a full day of in-person deep-phenotyping. This initial cohort was further divided into sub-cohort 1, utilized for training the machine-learning models, and a sub-cohort 2 used for validation of the models. The validation cohort consisted of 62 participants that also were part of the 52-week, double-blind, placebo-controlled, randomized clinical trial comparing phentermine-topiramate extended-release versus placebo (NCT04408586).

The second cohort consisted of 110 participants who underwent deep-phenotyping and were enrolled in a 16-week, double-blind, placebo-controlled, randomized clinical trial comparing liraglutide versus placebo⁶ (NCT03523273). This cohort served as the independent testing cohort. Upon excluding related samples and samples from outlier genetic backgrounds, the validation cohort for the genetic models was refined to a total of 50 samples.

### Study Protocol for Deep Phenotyping and Sample Collection, and Handling,

### In-Person Physiologic Testing Visit

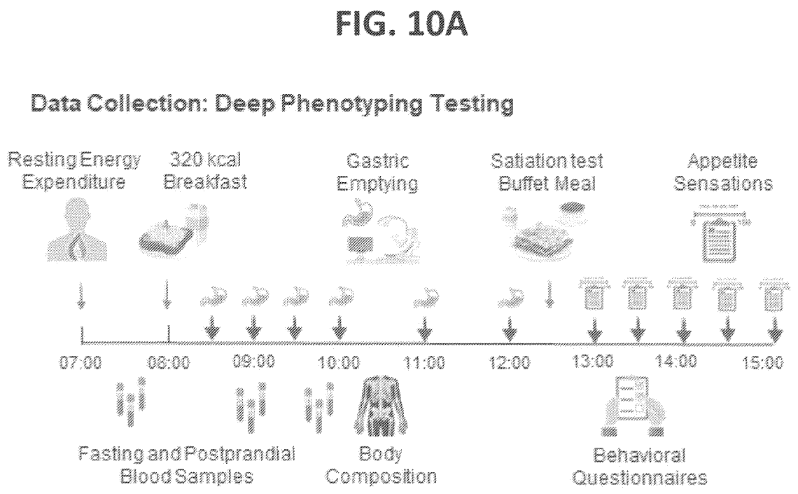

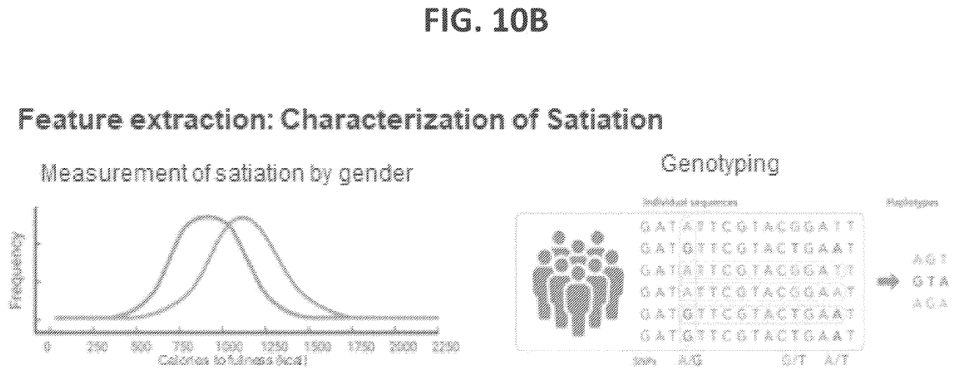

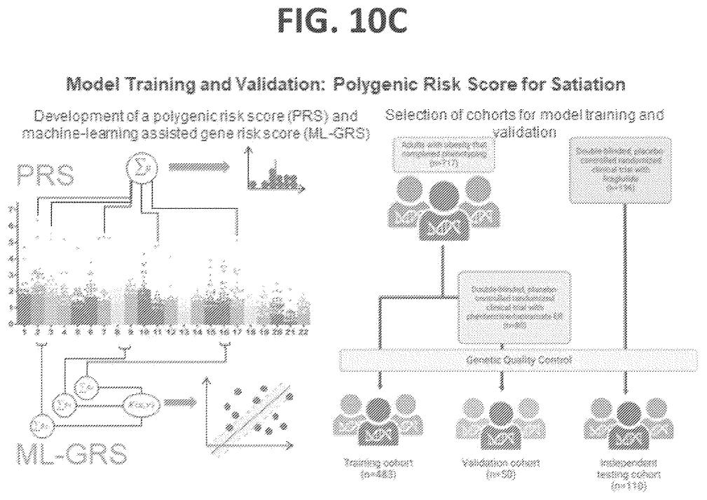

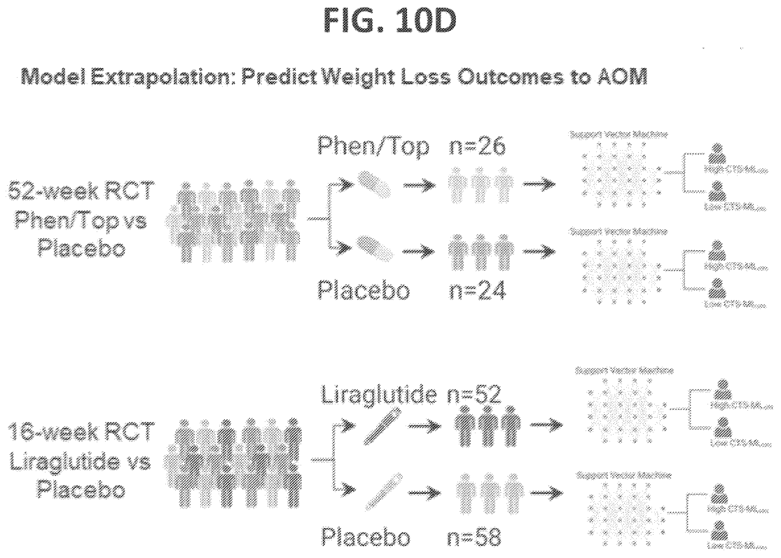

Participants underwent screening to meet inclusion and exclusion criteria and were sent a pre-visit study pack containing questionnaires (Hospital Anxiety and Depression Scale [HADS] questionnaire, Questionnaire on Eating and Weight Patterns, and the Three-Factor Eating Questionnaire [TFEQ-R21]) to complete at home. Upon arriving at the Clinical Research Trials Unit (CRTU) at Mayo Clinic, Rochester, MN, between 6:00 am and 8:00 am, participants were measured for heart rate and blood pressure while fasting. Standard clinical techniques were used to measure weight, height, hip, and waist circumference. Following the anthropometric measurements, participants underwent a baseline fasting blood draw, consumed a standardized radiolabeled breakfast to measure gastric emptying, and had an ad libitum meal for lunch (FIG. 10A).

Outcome Variables and Sample Collection. Handling, and Analysis

### Gastric Emptying for Solids

Participants received a standard breakfast comprising 320 kcal and 30% fat, including two 99mTc-radiolabeled eggs, toast, and 80 mL of skim milk. Images were taken immediately after ingestion and at regular intervals for a total of 4 hours. Gastric emptying (GE) was summarized by the half-emptying time (GE T1/2) in minutes.³⁸ (Camilleri et al. 2012).

### Fasting and Postprandial Gastric Volumes

Participants gastric volumes were measured by single photon emission computed tomography imaging (SPECT) of the stomach after intravenous injection of 99mTc-pertechnetate during fasting and after 300 mL Ensure drink³⁹.

### Satiation Test: Ad Libitum Meal

The ad libitum meal included: vegetable lasagna [Stouffers®, Nestle USA, Inc, Solon, OH; nutritional analysis of each 326 g box: 420 kcal, 17 g protein (16% of energy), 38 g carbohydrate (37% of energy), and 22 g fat (47% of energy)]; vanilla pudding [Hunts*. Kraft Foods North America, Tarrytown, NY; nutritional analysis of each 99 g carton: 130 kcal, lg protein (3% of energy), 21 g carbohydrate (65% of energy), and 4.5 g fat (32% of energy)]; and skim milk [nutritional analysis of each 236 mL carton: 90 kcal, 8 g protein (36% of energy), 13 g carbohydrate (64% of energy), and 0 g fat]. The total amount (g and kcal) consumed and the kcal of each macronutrient at the ad libitum meal were analyzed by a registered dietitian using validated software (ProNutra 3.0; Viocare Technologies Inc, Princeton, NJ). Participants were served the prepared meal. Subjects were asked to consume any food products until they achieved maximum satiation. A visual analog scale was used to assess maximal satiation (rate 0 to 5). As the subject completed each meal component, the empty plate, empty pudding container, and/or liquid volume of milk were weighed back. Once the subject reached maximum satiation, the total weight of food products consumed was summarized. The primary measurement was total kcal consumed to satiation (CTS).

### Eating Behaviors

The Three-Factor Eating Questionnaire (TFEQ-R21) is a validated questionnaire designed to assess eating and weight control behaviors. Participants completed the TFEQ-R21 on their baseline assessment visit. The TFEQ-R21 is a 21-item instrument that measures three domains of eating behavior: cognitive restraint (CR), uncontrolled eating (UE), and emotional eating (EE). The first twenty items are rated on a 4-point Likert scale, and item 21 is answered through an 8-point Likert scale. Before calculating domain scores, items 1-16 were reverse coded, and item 21 was recorded as follows: 1-2 scores as 1; 3-4 as 2; 5-6 as 3; 7-8 as 4. Domain scores were then calculated as a mean of all items within each domain; hence, domain scores ranged from 1 to 4 (CR [six items], UE [nine items], and EE [six items]), with higher scores being indicative of greater CR, UE, and EE.

### Sample Collection

Informed consent was obtained from all participants. Baseline blood samples following an overnight fast were collected from each subject into 6 ml EDTA blood collection tubes. Serum was kept at room temperature and immediately prepared for subsequent analysis of routine laboratory analyses or aliquoted and stored at −80° C. until further analyses.

### DNA Extraction

The samples were thawed at room temperature and a lysis buffer was added. The samples were then incubated at 56° C. for 10 minutes. Proteinase K was added, and the samples were incubated at 56° C. for an additional 30 minutes. Phenol/chloroform was added, and the samples were vortexed to mix. The samples were then centrifuged at 10,000 rpm for 10 minutes. The aqueous layer was transferred to a new tube and ethanol was added. The samples were vortexed to mix and then centrifuged at 10,000 rpm for 10 minutes. The supernatant was removed, and the pellet was air-dried. The pellet was then resuspended in the TE buffer. The DNA was purified using a commercial DNA purification kit. The kit included a lysis buffer, proteinase K, phenol/chloroform, and ethanol. The DNA was purified according to the manufacturer's instructions. The DNA was quantified using a NanoDrop spectrophotometer. The DNA concentration was determined by measuring the absorbance of the DNA at 260 nm.

### Metabolomics

Blood samples were collected from participants and stored at −80° C. until ready for analysis. The samples were thawed at room temperature and vortexed to mix. The samples were analyzed using liquid chromatography-mass spectrometry (LC-MS/MS). The samples were first separated on an LC column using a gradient elution method. The eluted compounds were then detected by mass spectrometry and the peak areas were quantified. The metabolites were identified by their mass-to-charge (m/z) ratios and retention times.

### Hormones

In this example, the levels of appetite-regulating hormones were measured, Peptide YY (PYY), glucagon-like peptide-1 (GLP-1), and ghrelin, in human blood samples using enzyme-linked immunosorbent assay (ELISA). ELISA is a highly sensitive and specific technique that allows for the quantification of target hormones in plasma. Monoclonal antibodies specific to PYY. GLP-1, and ghrelin were immobilized onto microtiter plates, and the blood samples were added to allow hormone binding. Subsequently, a second enzyme-linked antibody was added, initiating a color-producing reaction proportional to the hormone concentration.

### Genome-Wide Association Study (GWAS)

Genotyping was carried out using the Illumina Infinium BeadChip technology. Quality control measures were implemented to ensure data accuracy and reliability, and samples with low call rates or ambiguous results were excluded from further analysis. Prior to analysis, further metrics were implemented to ensure only accurate genotype calls were used. SNPs with a call rate <0.95 and samples with call rate <0.95 were removed. The relatedness between each sample was assessed using the kinship coefficient. One subject from the following related pairs was retained: twins, parent/offspring, and full siblings. The sample with the higher call rate was retained. Genomic ancestry was assessed using STRUCTURE. Non-White samples were removed (e.g., >20% non-White). Calls for SNPs with heterozygosity <1×10\hat{ }−6, MAF of 1%, and an information score of <0.X. SNP flips between samples were handled automatically by the analysis software.

P-values for each SNP were derived using a set of 497 samples. These samples have both genotyping data for 2.7 M SNPs typed using Illumina's Omni Exome array and CTS and exclude related samples and samples from outlier genetic backgrounds. Using PLINK 2¹¹, linear regression was performed using age as a covariate. Regression was performed separately for males and females as they have different CTS endpoints for high calories to satiation.

### Candidate Gene Selection & Polygenic Risk Score

A set of candidate genes to build a polygenic risk score were selected from a literature review based on their biological relevance to obesity and previous evidence from genetic studies (Table 5). Genes involved in appetite regulation, energy expenditure lipid metabolism, and adipogenesis were prioritized for inclusion in the study. This resulted in a set of 40 genes and 19,161 unique annotated variants, of which 2,637 were SNPs contained in the Omni Exome array. The weighted risk alleles were correlated with CTS for each SNP that had genetic variability across the 497 samples and selected the top 10% most correlated SNPS (n=227) as those most likely involved in appetite regulation. Subsequently, the PRS for each individual was calculated by summing the weighted risk alleles of the chosen SNPs.

TABLE 5
List of selected candidate genes for the development
of the polygenic risk score for satiation.

| | | | | # of |
|---|---|---|---|---|
| | | | Gene location | GRS |
| Number | Gene | Gene name | (GRCh38/hg38) | SNPs |
| 1 | ADRA2A | adrenoceptor alpha 2A | chr10: 111,077,029- | 1,268 |
| | | | 111,080,907 | |
| 2 | AGRP | agouti related neuropeptide | chr16: 67,482,571- | 875 |
| | | | 67,483,547 | |
| 3 | BDNF | Brain-derived neurotrophic | chr11: 27,654,893- | 1,035 |
| | | factor | 27,720,779 | |
| 4 | CADM2 | cell adhesion molecule 2 | chr3: 84,959,546-86,066,835 | 1,249 |
| 5 | FAIM2 | Fas apoptotic inhibitory | chr12: 49,866,896- | 1,067 |
| | | molecule 2 | 49,903,900 | |
| 6 | FANCL | FA complementation group L | chr2: 58,159,243-58,241,345 | 1,100 |
| 7 | FGFR4 | fibroblast growth factor | chr5: 177,086,915- | 1,592 |
| | | receptor 4 | 177,098,144 | |
| 8 | FTO | FTO alpha-ketoglutarate- | chr16: 53,704,156- | 1,873 |
| | | dependent dioxygenase | 54,121,941 | |
| 9 | GHRL | ghrelin and obestatin | chr3: 10,285,750-10,290,914 | 1,810 |
| | | prepropeptide | | |
| 10 | GIPR | gastric inhibitory polypeptide | chr19: 45,668,221- | 1,599 |
| | | receptor | 45,683,722 | |
| 11 | GLP1R | glucagon-like peptide 1 | chr6: 39,048,781-39,091,303 | 1,596 |
| | | receptor | | |
| 12 | GNB-3 | G protein subunit beta 3 | chr12: 6,840,925-6,847,393 | 1,689 |
| 13 | GNPDA2 | glucosamine-6-phosphate | chr4: 44,701,795-44,726,556 | 949 |
| | | deaminase 2 | | |
| 14 | GPRC5B | G protein-coupled receptor | chr16: 19,856,691- | 1,317 |
| | | class C group 5 member B | 19,884,848 | |
| 15 | HOXB5 | homeobox B5 | chr17: 48,591,257- | 1,000 |
| | | | 48,593,779 | |
| 16 | KCTD15 | potassium channel | chr19: 33,796,870- | 1,527 |
| | | tetramerization domain | 33,815,761 | |
| | | containing 15 | | |
| 17 | LEPR | leptin receptor | chr1: 65,420,668-65,641,559 | 1,395 |
| 18 | LINGO1 | leucine rich repeat and Ig | chr15: 77,613,670- | 865 |
| | | domain containing 1 | 77,696,148 | |
| 19 | MBOAT4 | membrane-bound O- | chr8: 30,131,671-30,144,665 | 1,305 |
| | | acyltransferase domain | | |
| | | containing 4 | | |
| 20 | MC4R | melanocortin 4 receptor | chr18: 60,371,062- | 1,228 |
| | | | 60,372,775 | |
| 21 | MTCH2 | mitochondrial carrier 2 | chr11: 47,617,317- | 755 |
| | | | 47,642,559 | |
| 22 | NCOA1 | nuclear receptor coactivator 1 | chr2: 24,492,058-24,770,702 | 1,234 |
| 23 | NEGR1 | neuronal growth regulator 1 | chr1: 71,395,943-72,282,539 | 1,398 |
| 24 | NPY1R | neuropeptide Y receptor Y1 | chr4: 163,323,962- | 1,350 |
| | | | 163,332,596 | |
| 25 | NPY2R | neuropeptide Y receptor Y2 | chr4: 155,208,636- | 1,363 |
| | | | 155,217,076 | |
| 26 | NPY5R | neuropeptide Y receptor Y5 | chr4: 163,350,219- | 1,347 |
| | | | 163,351,636 | |
| 27 | NR1H4 | nuclear receptor subfamily 1 | chr12: 100,473,708- | 1,137 |
| | | group H member 4 | 100,564,413 | |
| 28 | NUDT3 | nudix hydrolase 3 | chr6: 34,279,679-34,392,669 | 1,645 |
| 29 | OLFM4 | olfactomedin 4 | chr13: 53,028,813- | 1,185 |
| | | | 53,052,057 | |
| 30 | PCSK1 | proprotein convertase | chr5: 96,390,333-96,433,248 | 1,168 |
| | | subtilisin/kexin type 1 | | |
| 31 | POMC | proopiomelanocortin | chr2: 25,160,915-25,168,903 | 882 |
| 32 | PTBP2 | polypyrimidine tract binding | chr1: 96,721,783-96,823,738 | 1,153 |
| | | protein 2 | | |
| 33 | SH2B1 | S2HB adaptor protein 1 | chr16: 28,863,843- | 675 |
| | | | 28,874,205 | |
| 34 | SIM1 | SIM bHLH transcription | chr6: 100,385,009- | 1,213 |
| | | factor 1 | 100,464,921 | |
| 35 | TCF7L2 | transcription factor 7 like 2 | chr10: 112,950,664- | 1,129 |
| | | | 113,166,160 | |
| 36 | TFAP2B | transcription factor AP-2 beta | chr6: 50,818,871-50,847,619 | 924 |
| 37 | TMEM18 | Transmembrane protein 18 | chr2: 663,877-677,406 | 1,249 |
| 38 | TNFRSF11A | TNF receptor superfamily | chr18: 62,325,310- | 1,571 |
| | | member 11a | 62,391,288 | |
| 39 | TNNI3K | TNNI3 interacting kinase | chr1: 74,235,387-74,544,428 | 899 |
| 40 | UCP-3 | uncoupling protein 3 | chr11: 74,000,277- | 1,068 |
| | | | 74,009,085 | |
| 41 | UCP2 | uncoupling protein 2 | chr11: 73,974,672- | 1,094 |
| | | | 73,982,843 | |

### Gene Risk Score Development

In preparation for training a machine-learning gene risk score (ML-GRS) model, a method for the scoring of genes was created. This score numerically represents the overall contribution of variants in and around a candidate gene to CTS. To calculate this score, contributing factors at variants near the 40 candidate genes were investigated. These factors (deemed “contribution factors”) included the resulting beta value from linear regression analysis (using sex-specific endpoints) and other parameters such as the variant's proximity to the gene, and its apparent mechanistic role in gene. Integration of these factors (contribution factors) and SNP dosage information was performed across an extended region of one megabase pairs flanking the candidate genes, incorporating several hundred variants per gene (Table 6). This process resulted in two numerical representations of each gene contribution to CTS, each of which was termed a Gene Risk Score (GRS).

TABLE 6
Demographic characteristics and parameters of energy balance regulation
by low and high calories consumed to satiation (CTS).

| | High CTS | Low CTS | |
|---|---|---|---|
| | n = 179 | n = 161 | p-value |
| Age, years | 39.1 ± 10.6 | 43.2 ± 11.9 | <0.001 |
| Females, n (%) | 134 (75%) | 121 (75%) | 0.95 |
| Weight, kg | 109.6 ± 23.0 | 104.5 ± 22.7 | 0.04 |
| Height, cm | 170.6 ± 9.0 | 168.1 ± 8.9 | 0.01 |
| Harris-Benedict, kcal/day | 1938.6 ± 355.3 | 1855.2 ± 359.8 | 0.03 |
| BMI, kg/m2 | 37.6 ± 7.4 | 36.87 ± 6.7 | 0.31 |
| Systolic blood pressure, mmHg | 129.6 ± 14.8 | 132.5 ± 15.9 | 0.08 |
| Diastolic blood pressure, mmHg | 76.9 ± 10.8 | 81.3 ± 10.8 | 0.95 |
| Pulse, bpm | 72.2 ± 11.3 | 75.3 ± 12.3 | 0.02 |
| Waist circumference, cm | 113.0 ± 15.7 | 112.7 ± 15.1 | 0.89 |
| Hip circumference, cm | 124.6 ± 13.3 | 123.0 ± 13.1 | 0.26 |
| Waist/hip, ratio | 0.90 ± 0.08 | 0.92 ± 0.08 | 0.25 |
| Gastric emptying 120 min, ratio | 0.44 ± 0.16 | 0.50 ± 0.16 | <0.001 |
| Gastric emptying T 1/2, min | 121.9 ± 28.9 | 130.8 ± 32.4 | 0.008 |
| Calories to satiation, kcal | 1293.9 ± 260.0 | 618.1 ± 116.8 | <0.001 |
| VAS-hunger before satiation test, | 66.5 ± 22.2 | 53.7 ± 24.9 | <0.001 |
| mm | | | |
| VAS-fullness before satiation test, | 15.1 ± 15.4 | 30.2 ± 21.5 | <0.001 |
| mm | | | |
| VAS-hunger 2 hours CTS, mm | 23.0 ± 20.7 | 23.4 ± 21.9 | 0.91 |
| VAS-Fullness 2 hours CTS, mm | 68.3 ± 23.2 | 67.7 ± 24.6 | 0.85 |
| Lean Mass, % | 53.1 ± 7.3 | 53.0 ± 5.9 | 0.90 |
| Fat Mass, % | 0.48 ± 0.07 | 0.48 ± 0.06 | 0.87 |
| Diabetes, n (%) | 15 (8.57) | 11 (6.47) | 0.25 |
| Hx of prior use of AOM, n (%) | 43 (25) | 40 (23.5) | 0.26 |

Abbreviations used: AOM, anti-obesity medications; BMI, body mass index; CTS, calories to satiation; Hx, history; VAS, visual analog scale. Categorical variables were compared with Fisher's exact tests and continuous variables with independent t tests. Continuous variables are presented as mean ± standard deviation.

Further preparations for training the ML-GRS were done by adding other data sources and retaining only the most informative candidate GRSs and other data sources (together ‘features’). The other data sources considered were demographic and anthropometric sources (e.g., age, sex, weight). Forward feature selection was used identify the most informative features. In brief, starting with a single feature, the feature with the best training accuracy using a random forest model was selected. Then the remaining feature with the best training accuracy when combined with the previously selected feature(s) were selected. This was continued until no accuracy improvement was observed. This method selected height, weight, age, sex, and 16 genetic risk scores associated with 10 genes: SIM1*, PCSK1, SH2B1. LEPR, UCP2*, FTO*, TCF7L2*, GLP1R, TNFRSF11A, and ADRA2A. Genes marked with an asterisk had only the GRS derived from the female endpoint selected. Note that because of the algorithm's greedy approach, features were selected when they were both predictive and contributed novel information. Thus, some expected genes, such as MC4R, were not selected because functionally related genes already contained similar predictive information, not because such genes were not predictive of CTS.

Scores for selected features were generated for all samples with both genetic information and CTS: 50 samples in the Phentermine/Topiramate trial; 110 in the Liraglutide trial; and 483 that participated in neither trial.

### Machine-Learning Model Training to Predict High Calories to Satiation

The ML-GRS was trained in 483 samples using a Support Vector Classifier (SVC) with the 16 selected GRSs, height, weight, age, and sex to predict high calories to satiation positive individuals (HS+). HS+ was defined as those individuals whose CTS was above the sex-specific 75th % ile (Male=1372.4275, Female=977.3325) and the others as HS−. A binary indicator of sex was then included along with the genetic risk scores. Before training, the scores were normalized using a Yeo power transform41 and conducted SMOTE42 sampling to create synthetic samples for training. An SVC model 10-fold cross-validation on these oversampled data was trained, with each fold's test data normalized based on the training data from that fold. A mean AUC=0.82 was measured across the 10 folds. The final SVC model used all the training samples. All SVC models were trained using the Python skleam.svm package with a linear kernel, C=3, gamma=0.01, and a balanced class weight. The final model used oversampling and normalization for all 483 training samples and yielded both an HS+/− prediction and a probability of HS+(p(HS+)).

### Machine-Learning Model Validation

To evaluate the model, the performance of the final SVC satiation model's prediction accuracy was tested on two placebo-controlled trials. These samples include 50 from a 52-week phentermine/topiramate trial (Test) which also underwent calories to satiation testing and had genotyping data. These samples' data were normalized the same as the training and the HB+ class was set using the same CTS thresholds. The outcomes of the samples of the treatment arm of the study was also evaluated for those predicted to HS+ and HS− using a threshold of p(HS+)>0.5 to define HS+. The Total Body Weight Loss of individuals was measured at 12-, 26-, 36-, and 52 weeks in each group under the hypothesis that predicted HS+ individuals are better responders (>15% TBWL at 52 weeks). Comparison of predicted and actual HS+/− shown in shown in Table 7A.

### Machine-Learning Model Independent Testing and Validation

The performance of the final SVC model's predictions on the Liraglutide trial samples was then measured following the same procedures as the validation. These samples include 110 from a 16-week liraglutide trial which also have genotyping data and calories to satiation in order to evaluate the outcomes of the samples of the treatment arm of the study for those predicted to HS+ and HS− using a threshold of p(HS+)<0.5 to define HS+. The Total Body Weight Loss of individuals at 5- and 16-weeks in each group were measured under the hypothesis that, in this case, predicted HB− individuals are instead better responders (>4% TBWL at 16-weeks). Comparison of predicted and actual HS+/− shown in shown in Table 13B.

TABLE 7A-B
Performance of the CTS_{GRS} predictions for characterization
of responders, defined by a total body weight loss percentage
of at least A) 15% in participants assigned to phentermine-
topiramate extended release at 52 weeks, and B) 4% in participants
assigned to liraglutide at 16 weeks.
A: Phentermine-Topiramate

| | | Response | | |
|---|---|---|---|---|
| | | (TBWL > 15%) | | |
| | | Yes | No | Total |
| Predicted | High | 4 | 5 | 9 |
| CTS_{GRS} | Low | 1 | 9 | 10 |

| | | | |
|---|---|---|---|
| Total | 5 | 14 | 19 |

B: Liraglutide

| | | Response | | |
|---|---|---|---|---|
| | | (TBWL > 4%) | | |
| | | Yes | No | Total |
| Predicted | High | 9 | 11 | 20 |
| CTS_{GRS} | Low | 23 | 4 | 27 |

| | | | |
|---|---|---|---|
| Total | 32 | 15 | 47 |

### Statistical Analysis

Continuous data were summarized as mean and standard deviation, and categorical variables as percentages. Statistical testing included two-sample unpaired t-tests for Gaussian distribution and Mann-Whitney tests for non-Gaussian distribution. To explore the variability between satiation, as measured by the kilocalories consumed at an ad libitum meal to satiation, and a comprehensive set of exposure variables including demographics (such as sex, age, and race), body composition (comprising fat mass, lean mass, and fat-free mass), anthropometric measures (including weight, height, waist circumference, and hip circumference), hormones (including PPY, GLP-1, and ghrelin levels), electronic medical record-derived data (about medication usage and comorbidities), and responses to behavioral questionnaires, a multivariable regression approach was employed. The primary outcome of the analysis was the coefficient of determination (R2) to quantify the proportion of variability in satiation explained by these diverse factors. To assess the precision of the R2 estimates, the standard error was calculated using the formula SER2=((4R2 (1−R2)2 (n−k−1)2)/((n2−1) (3+n)))1/2, where n represents the sample size and k represents the number of degrees of freedom in the model. Additionally, 95% confidence intervals for R2 were reported, which were computed as R2+/−1.96 times the standard error.

After stratifying participants by treatment allocation, independent t-tests were conducted at each timepoint to evaluate differences in total body weight loss across different groups (high CTS vs. low CTS and high CTS_{GRS} vs. low CTS_{GRS}). No method for data imputation for missing weight loss outcomes at different time points was used. The final ML-GRS model to predict CTS (CTS_{GRS}), using all training samples, provided a continuous value ranging from 0 to 1, indicating the probability of having high CTS. Participants were dichotomously classified as having high if the probability was equal to or exceeded 0.5. The AUC was estimated by using the Wilson-Brown method for both CTS and CTS_{GRS} values in predicting the probability of achieving a weight loss equal to or greater than 4% and 15% of TBWL for liraglutide and phentermine/topiramate, respectively. To evaluate the real-world implications of the model's predictions, t-tests were conducted to compare variables such as caloric intake, gastric emptying, and physiologic testing results among participants grouped by their predicted satiation groups.

JMP Pro (Version 16.2.0, SAS Institute Inc, Cary. NC) and GraphPad Prism (Version 10.0. GraphPad Software, Boston. MA) was employed for statistical analysis and visualization. Genome-wide association analysis was conducted using PLINK2. All models were developed and trained using Python.

### Results

### Cohort Measurements and Heterogeneity of Satiation

In this comprehensive study, the factors influencing the heterogeneity of satiation. The baseline characteristics of adult participants with obesity (Body Mass Index [BMI]>30 kg/m²) recruited between 2010 and 2021, all of whom underwent a comprehensive in-person deep-phenotyping with physiological and behavioral assessments to study components of energy balance regulation were examine (FIG. 10A). The deep-phenotype testing day started after an overnight fast and included the measurement of resting energy expenditure by indirect calorimetry, body composition by dual x-ray absorptiometry, the collection of blood samples, administration of a standardized radio-labeled 320-kcal breakfast to assess gastric emptying by scintigraphy and appetite sensations scales, measurement of satiation and behavioral questionnaires (FIG. 10A). In the satiation test, participants consumed an ad libitum meal consisting of vegetable lasagna, vanilla pudding, and skim milk. Nutritional analyses of the meal components were conducted, and participants were instructed to eat until they reached satiation, i.e., maximal fullness, assessed using a visual analog scale (VAS). The total kcal consumed to satiation (CTS) was the primary measurement. During the study day, blood samples were collected to extract DNA and plasma to measure fasting and postprandial gastrointestinal hormones (FIG. 10A).

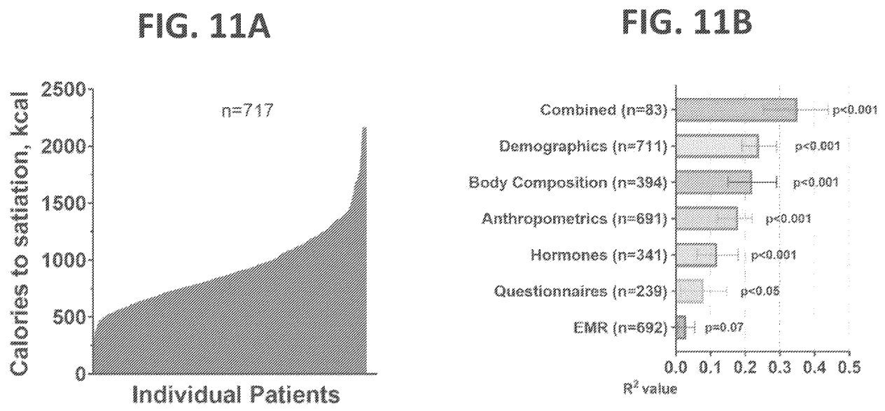

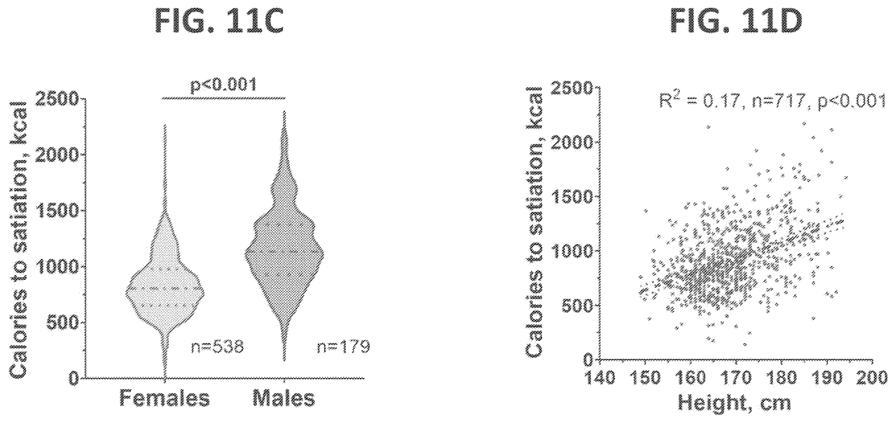

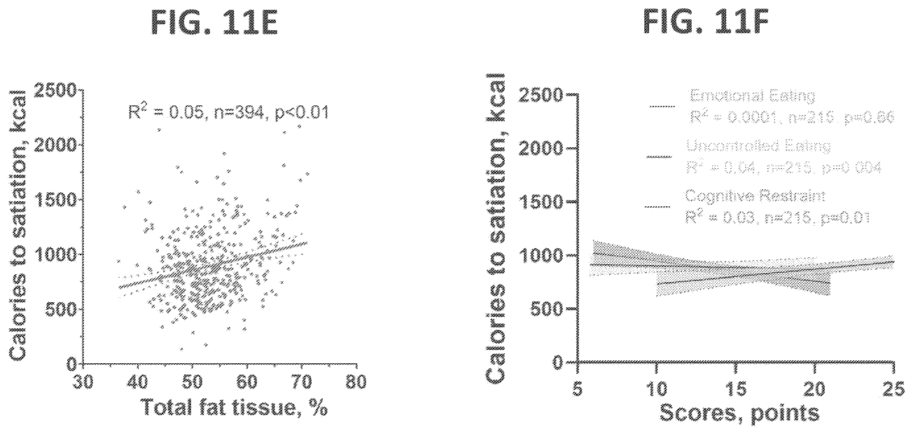

The initial study cohort comprised a total of 717 participants that completed the full day of in-person deep-phenotyping. Participants were mostly middle-aged females (75%) and White (91.9%) with a mean age of 41.1 and standard deviation (±) 11.4 years and a mean BMI of 37.0±7.1 kg/m² (Table 8). Females had a higher BMI when compared to males (37.3±7.0 kg/m² vs 35.9±7.4 kg/m², p=0.03). We observed satiation variability in our cohort, with a broad distribution among patients, ranging from 140 kcal to 2166 kcal to satiation (FIG. 11A).

TABLE 8
Demographic characteristics and parameters of energy balance regulation by sex,

| | All participants | Females | Males | |
|---|---|---|---|---|
| | 717 | 538 | 179 | p-value |
| Age, years | 41.1 ± 11.4 | 41.4 ± 11.0 | 40.3 ± 12.4 | 0.32 |
| Weight, kg | 105.6 ± 22.9 | 102.4 ± 20.8 | 115.0 ± 26.1 | <0001 |
| Height, cm | 168.9 ± 8.8 | 165.6 ± 6.6 | 178.7 ± 7.1 | <.0001 |
| Harris-Benedict, kcal/day | 1877.6 ± 348.2 | 1747.4 ± 210.6 | 2269.0 ± 385.4 | <.0001 |
| BMI, kg/m2 | 37.0 ± 7.1 | 37.3 ± 7.0 | 35.9 ± 7.4 | 0.03 |
| Systolic blood pressure, | 130.3 ± 14.8 | 128.4 ± 14.6 | 136.1 ± 14.0 | <0001 |
| mmHg | | | | |
| Diastolic blood pressure, | 79.1 ± 10.8 | 78.5 ± 10.9 | 81.2 ± 10.5 | 0.003 |
| mmHg | | | | |
| Pulse, bpm | 73.2 ± 11.7 | 74.1 ± 11.32 | 70.6 ± 12.6 | 0.001 |
| Waist circumference, cm | 111.2 ± 15.2 | 110.0 ± 14.6 | 114.7 ± 16.4 | 0.001 |
| Hip circumference, cm | 122.9 ± 13.3 | 124.3 ± 13.3 | 118.6 ± 12.3 | <.0001 |
| Waist/hip, ratio | 0.90 ± 0.08 | 0.88 ± 0.07 | 0.96 ± 0.07 | <.0001 |
| Gastric emptying 120 | 0.46 ± 0.17 | 0.50 ± 0.15 | 0.36 ± 0.16 | <0001 |
| min, ratio | | | | |
| Gastric emptying T 1/2, | 127.0 ± 32.7 | 132.7 ± 31.5 | 109.9 ± 30.3 | <0001 |
| min | | | | |
| Calories to satiation, kcal | 917.4 ± 315.4 | 835.2 ± 259.1 | 1164.2 ± 340.5 | <.0001 |
| VAS-hunger 2 hours | 24.4 ± 22.0 | 23.8 ± 22.1 | 26.3 ± 21.5 | 0.32 |
| CTS, mm | | | | |
| VAS-Fullness 2 hours | 67.5 ± 23.4 | 68.2 ± 23.2 | 64.8 ± 23.8 | 0.21 |
| CTS, mm | | | | |
| VAS-satisfaction 2 hours | 66.5 ± 24.6 | 65.9 ± 25.1 | 68.3 ± 22.8 | 0.37 |
| CTS, mm | | | | |
| Total Lean Mass, kg | 54.7± 10.7 | 50.5 ± 6.5 | 68.9 ± 9.8 | <.0001 |
| Lean Mass, % | 53.1 ± 6.2 | 51.1 ± 4.7 | 59.7 ± 6.0 | <0001 |
| Fat Mass, % | 0.5 ± 0.1 | 0.5 ± 0.1 | 0.41 ± 0.1 | <0001 |
| Hx of prior use of AOM, | 149 (20.8) | 117 (21.7) | 32 (17.9) | 0.25 |
| n (%) | | | | |
| Diabetes, n (%) | 43 (6.0) | 26 (4.8) | 17 (9.5) | 0.03 |

Abbreviations used: AOM, anti-obesity medications; BMI, body mass index; CTS, calories to satiation; Hx, history; VAS, visual analog scale. Categorical variables were compared with Fisher's exact tests and continuous variables with independent t tests. Continuous variables are presented as mean ± standard deviation.

To explore the factors contributing to the variability in satiation measurements, separate multivariable linear regression models incorporating diverse input variables were conducted. These variables encompassed demographic characteristics (i.e., age, sex, and race), anthropometrics (i.e., weight, height, waist circumference, and hip circumference) body composition (i.e., fat mass, lean mass, and fat free mass), hormones (i.e., peptide YY [PYY], ghrelin, and glucagon-like peptide 1 [GLP-1]), questionnaires (i.e., Three-Factor Eating Questionnaire [TFEQ], Hospital Anxiety and Depression Scale (HADS), Alcohol Use Disorders Identification Test, and Weight Efficacy Lifestyle Questionnaire) and parameters extracted from electronic medical records (i.e., past medical history of: diabetes, hypothyroidism, gastroesophageal reflux, metabolic dysfunction-associated steatosis liver disease, smoking, and history of use of anti-obesity medications). In addition, we conducted a model including demographic, anthropometric, body composition, and hormone features was conducted (Table 9A-C).

TABLE 9A-C
(A) Individual features included in the multivariate models. B) Parameter
estimates of multivariable linear regression models to estimate calories
to satiation during an ad libitum buffet meal. C) Model characteristics.
A. Individual characteristic regression inputs

| | | | | | | Electronic |
|---|---|---|---|---|---|---|
| | | | | | Body | Medical |
| Combined | Demographic | Anthropometric | Questionnaires | Hormones | Composition | Record |
| (n = 88) | (n = 711) | (n = 711) | (n = 216) | (n = 314) | (n = 394) | (n = 692) |
| Age (years) | Age (years) | Weight (kg) | Three-Factor | Plasma | Total lean | History of |
| | | | Eating | Ghrelin | mass (gm) | diabetes |
| | | | Questionnaire, | at | | (Yes, No) |
| | | | emotional | fasting, | | |
| | | | eating | 15, 45, | | |
| | | | component | and 90 | | |
| | | | (points) | minutes | | |
| | | | | (ug/dL) | | |
| Sex (M/F) | Sex (M/F) | Height (m) | Three-Factor | Plasma | Total fat- | History of |
| | | | Eating | PYY at | free mass | hypothyroidism |
| | | | Questionnaire, | fasting, | (gm) | (Yes, No) |
| | | | cognitive | 15, 45, | | |
| | | | restraint | and 90 | | |
| | | | component | minutes | | |
| | | | (points) | (ug/dL) | | |
| Race (White, | Race | Hip | Three-Factor | Plasma | Total Tissue | History of |
| black, asian, | (White, | circumference | Eating | GLP-1 at | Fat (%) | gastro- |
| other) | black, asian, | (cm) | Questionnaire, | fasting, | | esophageal |
| | other) | | uncontrolled | 15, 45, | | reflux |
| | | | eating | and 90 | | (Yes, No) |
| | | | component | minutes | | |
| | | | (points) | (ug/dL) | | |
| Weight (kg) | | Waist | Hospital | | | History of |
| | | circumference | Anxiety and | | | metabolic |
| | | (cm) | Depression | | | dysfunction- |
| | | | Scale, | | | associated |
| | | | Depression | | | steatosis liver |
| | | | component | | | disease (Yes, |
| | | | (Points) | | | No) |
| Height (m) | | | Hospital | | | History of use |
| | | | Anxiety and | | | of anti-obesity |
| | | | Depression | | | medications |
| | | | Scale, Anxiety | | | (Yes, No) |
| | | | component | | | |
| | | | (Points) | | | |
| Hip | | | Weight | | | History of |
| circumference | | | Efficacy | | | smoking |
| (cm) | | | Lifestyle | | | (Yes, No) |
| | | | Questionnaire, | | | |
| | | | Physical | | | |
| | | | Discomfort | | | |
| | | | (points) | | | |
| Waist | | | Weight | | | |
| circumference | | | Efficacy | | | |
| (cm) | | | Lifestyle | | | |
| | | | Questionnaire, | | | |
| | | | Social | | | |
| | | | Pressure | | | |
| | | | (points) | | | |
| Plasma PYY | | | Weight | | | |
| at fasting, | | | Efficacy | | | |
| 15, 45, and | | | Lifestyle | | | |
| 90 minutes | | | Questionnaire, | | | |
| (ug/dL) | | | Positive | | | |
| | | | Activities | | | |
| | | | (points) | | | |
| Plasma GLP- | | | Weight | | | |
| 1 at fasting, | | | Efficacy | | | |
| 15, 45, and | | | Lifestyle | | | |
| 90 minutes | | | Questionnaire, | | | |
| (ug/dL) | | | Negative | | | |
| | | | Emotions | | | |
| | | | (Points) | | | |
| Total Tissue | | | Weight | | | |
| Fat (%) | | | Efficacy | | | |
| | | | Lifestyle | | | |
| | | | Questionnaire, | | | |
| | | | Availability | | | |
| | | | (Points) | | | |
| | | | Alcohol Use | | | |
| | | | Disorders | | | |
| | | | Identification | | | |
| | | | Test (Points) | | | |

B. Parameter estimates for each model

| | | | |
|---|---|---|---|
| Parameter | Estimate | Std Error | Prob > \|t\| |

Combined

| | | | |
|---|---|---|---|
| Age at study, years | −1.22 | 3.46 | 0.72 |
| Sex, Female | −13.74 | 73.39 | 0.85 |
| Race, Non-White | −37.45 | 100.35 | 0.71 |
| Height, m | 601.17 | 639.05 | 0.35 |
| Weight, kg | 5.96 | 4.01 | 0.14 |
| Waist circumference, cm | 0.01 | 4.71 | 1.00 |
| Hip circumference, cm | −2.25 | 7.53 | 0.77 |
| Plasma GLP-1 AUC, ug/dL | 0.07 | 0.10 | 0.48 |
| Plasma PYY AUC, ug/dL | 0.00 | 0.01 | 0.69 |
| Total Tissue Fat, % | −1432.83 | 1130.97 | 0.21 |

Demographics

| | | | |
|---|---|---|---|
| Age at study, years | −3.49 | 0.91 | 0.0001* |
| Sex, Female | −170.26 | 12 | <.0001* |
| Race, Asian | −170.52 | 63.63 | 0.008* |
| Race, Black | −65.86 | 55.87 | 0.24 |
| Race, Other | 92.12 | 48.95 | 0.06 |

Anthropometrics

| | | | |
|---|---|---|---|
| Height, m | 997.99 | 165.41 | <.0001* |
| Weight, kg | 4.48 | 1.2 | 0.0002* |
| Waist circumference, cm | −1.08 | 1.39 | 0.47 |
| Hip circumference, cm | −5.69 | 1.6 | 0.0004* |

Hormones

| | | | |
|---|---|---|---|
| Plasma Ghrelin fasting, ug/dL | −0.29 | 0.54 | 0.59 |
| Plasma Ghrelin 15 mint, ug/dL | 0.33 | 0.52 | 0.53 |
| Plasma Ghrelin 45 min, ug/dL | −0.50 | 0.94 | 0.6 |
| Plasma Ghrelin 90 min, ug/dL | −1.04 | 1.02 | 0.31 |
| Plasma GLP-1 fasting, ug/dL | 10.85 | 9.96 | 0.28 |
| Plasma GLP-1 15 min, ug/dL | −3.62 | 7.25 | 0.62 |
| Plasma GLP-1 45 min, ug/dL | 3.009 | 3.4 | 0.38 |
| Plasma GLP-1 90 min, ug/dL | −3.11 | 3.52 | 0.38 |
| Plasma PYY fasting, ug/dL | 1.57 | 1.51 | 0.3 |
| Plasma PYY 15 min, ug/dL | −0.39 | 1.55 | 0.8 |
| Plasma PYY 45 min, ug/dL | −3.59 | 0.94 | 0.0002* |
| Plasma PYY 90 min, ug/dL | 2.54 | 0.61 | <.0001* |

Body Composition

| | | | |
|---|---|---|---|
| Total lean mass, gm | −0.02 | 0.06 | 0.67 |
| Total fat-free mass, gm | 0.04 | 0.05 | 0.51 |
| Total Tissue Fat, % | −442.28 | 250.9 | 0.09 |

Questionnaires

| | | | |
|---|---|---|---|
| Three-Factor Eating Questionnaire - | −13.80 | 9.6 | 0.15 |
| cognitive restraint component, points | | | |
| Three-Factor Eating Questionnaire - | 14.16 | 6.77 | 0.04* |
| uncontrolled eating component, points | | | |
| Weight Efficacy Lifestyle | −11.66 | 5.74 | 0.04* |
| Questionnaire - Negative Emotions, | | | |
| Points | | | |
| Weight Efficacy Lifestyle | −0.84 | 6.25 | 0.89 |
| Questionnaire - Availability, Points | | | |
| Weight Efficacy Lifestyle | −4.84 | 4.93 | 0.33 |
| Questionnaire - Social Pressure, | | | |
| points | | | |
| Weight Efficacy Lifestyle | 8.42 | 5.95 | 0.16 |
| Questionnaire - Physical Discomfort, | | | |
| points | | | |
| Weight Efficacy Lifestyle | 8.43 | 5.79 | 0.15 |
| Questionnaire - Positive Activities, | | | |
| points | | | |
| Alcohol Use Disorders Identification | 8.41 | 17.79 | 0.64 |
| Test, Points | | | |
| Hospital Anxiety and Depression | −14.81 | 9.96 | 0.14 |
| Scale -Anxiety component, Points | | | |
| Hospital Anxiety and Depression | 15.57 | 10.97 | 0.16 |
| Scale -Depression component, Points | | | |
| Three-Factor Eating Questionnaire - | −19.16 | 10.04 | 0.06 |
| emotional eating component, points | | | |

Electronic Medical Record

| | | | |
|---|---|---|---|
| History of gastro-esophageal reflux, | 6.38 | 18.23 | 0.73 |
| No | | | |
| History of metabolic dysfunction- | −6.88 | 37.09 | 0.85 |
| associated steatosis liver disease, No | | | |
| History of diabetes, No | −63.78 | 25.25 | 0.0118* |
| History of use of anti-obesity | 11.2 | 20.84 | 0.59 |
| medications, No | | | |
| History of hypothyroidism, No | 35.42 | 18.26 | 0.05 |
| History of smoking, No | −14.24 | 22.56 | 0.53 |

C- Model characteristics

| | | | | Upper | |
|---|---|---|---|---|---|
| | Sample | Adjusted | Lower Limit | Limit | |
| | Size | R squared | 95% CI | 95% CI | p-value |
| Combined | 88 | 0.35 | 0.45 | 0.25 | 0.001 |
| Demographics | 711 | 0.24 | 0.19 | 0.29 | 0.001 |
| Anthropometrics | 691 | 0.17 | 0.12 | 0.22 | 0.001 |
| Hormones | 341 | 0.12 | 0.06 | 0.18 | 0.001 |
| Body Composition | 394 | 0.22 | 0.15 | 0.29 | 0.001 |
| Questionnaires | 239 | 0.05 | 0.03 | 0.15 | 0.05 |
| Electronic Medical | 692 | 0.02 | 0.005 | 0.05 | 0.07 |
| Record | | | | | |

The analysis revealed that demographic characteristics, specifically sex, race, and age, accounted for the highest proportion of variance in satiation (r²=0.24; p=0.001) (Table 9A-C; FIG. 11B). Despite these associations being statistically significant, the overall strength of the relationships remained weak across all models. Notably, a significant gender difference emerged, with females requiring fewer calories to reach satiation compared to males (835.2±259.1 kcal vs. 1164.2±340.5 kcal, p<0.001) (FIG. 11C). Anthropometric variables exhibited a variance of r²=0.17 (p=0.001), with height emerging as the most influential contributor to satiation (r²=0.17, p=0.001) within this category, although the associations remained relatively weak (FIG. 11D).

In the assessment of body composition, fat percentage demonstrated the strongest association with satiation among other parameters (see Table 9A-C). However, this association was still notably weak (r²=0.05, p<0.01; FIG. 11E).

Despite efforts to incorporate validated questionnaires, such as the TFEQ, into the analysis, it became evident that these components alone were insufficient to fully elucidate the variability in satiation (FIG. 11F). Similarly, a high degree of variability was observed in gastrointestinal hormonal profiles, with limited associations with satiation, as demonstrated in Table 10. Finally, a model incorporating demographic, anthropometric, body composition and hormone features together had a modest association with satiation (r²=0.35; p<0.001).

TABLE 10
Distribution of CCK, Ghrelin, GLP-1, and PPY at −15 (fasting) and 15, 45, and 90 minutes
after a standard 320 kcal breakfast, divided by gender and correlated to calories to satiation.

| | Total | | | | Female | | | | Male | | | |
|---|---|---|---|---|---|---|---|---|---|---|---|---|
| | | Mean ± | | p- | | Mean ± | | p- | | Mean ± | | p- |
| | N | SD | R | value | N | SD | R | value | N | SD | R | value |
| CCK at −15 min, | 255 | 0.35 ± | 0 | 0.95 | 177 | 0.33 ± | 0.04 | 0.63 | 78 | 0.38 ± | −0.11 | 0.34 |
| ug/dL | | 0.32 | | | | 0.2 | | | | 0.49 | | |
| CCK at 15 min, | 255 | 4.31 ± | −0.06 | 0.34 | 177 | 4.31 ± | −0.03 | 0.65 | 78 | 4.32 ± | −0.12 | 0.31 |
| ug/dL | | 3.87 | | | | 3.57 | | | | 4.49 | | |
| CCK at 45 min, | 255 | 7.45 ± | 0.03 | 0.68 | 177 | 7.42 ± | 0.03 | 0.72 | 78 | 7.52 ± | 0.02 | 0.87 |
| ug/dL | | 5.25 | | | | 5.62 | | | | 4.3 | | |
| CCK at 90 min, | 253 | 6.25 ± | 0.24 | <.0001 | 175 | 5.88 ± | 0.2 | 0.01 | 78 | 7.09 ± | 0.25 | 0.03 |
| ug/dL | | 5.12 | | | | 4.48 | | | | 6.29 | | |
| Ghrelin at −15 min, | 339 | 70.22 ± | −0.04 | 0.49 | 243 | 71.57 ± | −0.04 | 0.5 | 96 | 66.82 ± | 0.01 | 0.9 |
| ug/dL | | 51.32 | | | | 47.24 | | | | 60.6 | | |
| Ghrelin at 15 min, | 341 | 65.3 ± | −0.03 | 0.63 | 243 | 65.85 ± | −0.1 | 0.11 | 98 | 63.93 ± | 0.13 | 0.19 |
| ug/dL | | 59.33 | | | | 60.58 | | | | 56.37 | | |
| Ghrelin at 45 min, | 341 | 45.45 ± | −0.06 | 0.23 | 244 | 46.95 ± | −0.08 | 0.22 | 97 | 41.69 ± | 0.07 | 0.47 |
| ug/dL | | 30.22 | | | | 32.1 | | | | 24.63 | | |
| Ghrelin at 90 min, | 338 | 39.13 ± | −0.12 | 0.03 | 241 | 40.86 ± | −0.11 | 0.09 | 97 | 34.82 ± | 0 | 0.97 |
| ug/dL | | 22.97 | | | | 23.57 | | | | 20.89 | | |
| GLP-1 at −15 min, | 335 | 4.72 ± | 0.06 | 0.24 | 237 | 4.56 ± | 0.06 | 0.38 | 98 | 5.12 ± | −0.04 | 0.69 |
| ug/dL | | 2.55 | | | | 2.4 | | | | 2.86 | | |
| GLP-1 at 15 min, | 345 | 6.56 ± | 0.02 | 0.75 | 245 | 6.44 ± | 0.01 | 0.82 | 100 | 6.85 ± | −0.04 | 0.68 |
| ug/dL | | 3.69 | | | | 3.59 | | | | 3.92 | | |
| GLP-1 at 45 min, | 346 | 13.09 ± | 0.05 | 0.31 | 246 | 12.9 ± | 0.05 | 0.4 | 100 | 13.56 ± | 0.03 | 0.76 |
| ug/dL | | 9.33 | | | | 9.22 | | | | 9.63 | | |
| GLP-1 at 90 min, | 342 | 13.48 ± | 0.16 | 0.004 | 242 | 12.84 ± | 0.14 | 0.03 | 100 | 15.01 ± | 0.11 | 0.26 |
| ug/dL | | 11.42 | | | | 10.71 | | | | 12.9 | | |
| PYY at −15 min, | 341 | 78.72 ± | 0.05 | 0.4 | 243 | 78.08 ± | 0.07 | 0.31 | 98 | 80.32 ± | −0.02 | 0.85 |
| ug/dL | | 24.63 | | | | 22.68 | | | | 28.96 | | |
| PYY at 15 min, | 339 | 87.91 ± | 0.01 | 0.86 | 241 | 87.27 ± | 0.02 | 0.72 | 98 | 89.48 ± | −0.05 | 0.62 |
| ug/dL | | 28.07 | | | | 26.07 | | | | 32.58 | | |
| PYY at 45 min, | 338 | 119.41 ± | 0.01 | 0.87 | 242 | 119.98 ± | 0.09 | 0.15 | 96 | 117.98 ± | −0.13 | 0.21 |
| ug/dL | | 45.46 | | | | 46.92 | | | | 41.75 | | |
| PYY at 90 min, | 335 | 139.83 ± | 0.18 | <0.001 | 238 | 136.99 ± | 0.26 | <.0001 | 97 | 146.81 ± | 0.04 | 0.67 |
| ug/dL | | 67.52 | | | | 64.04 | | | | 75.28 | | |

R represents the correlation from a simple linear regression analysis, demonstrating the relationship between individual time points for hormones and calories required for satiation. The p-values in the table were calculated to assess the strength and significance of this association.

### Characterizing High and Low Calories Consumed to Satiation

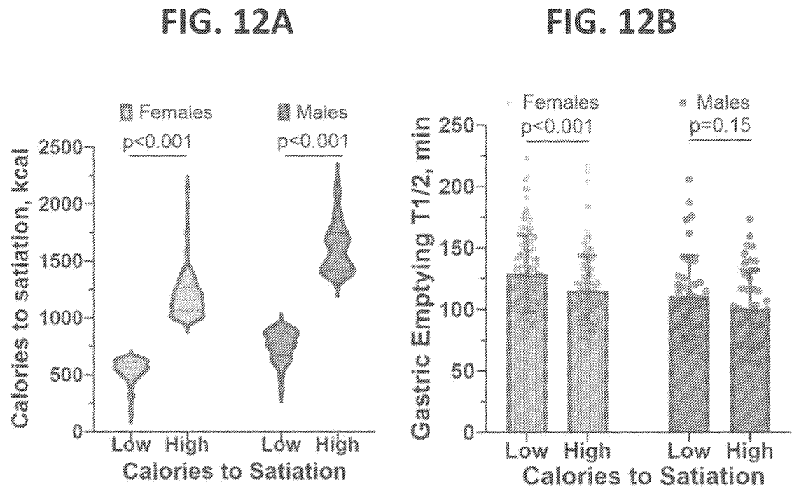

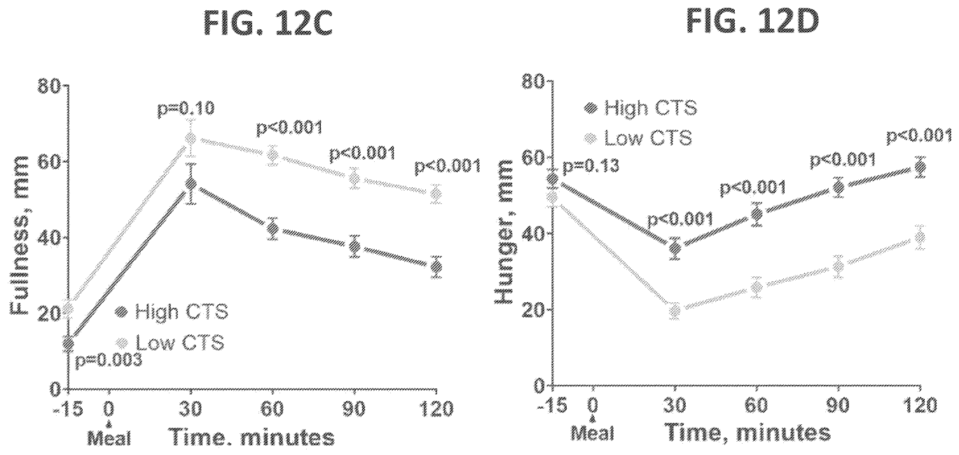

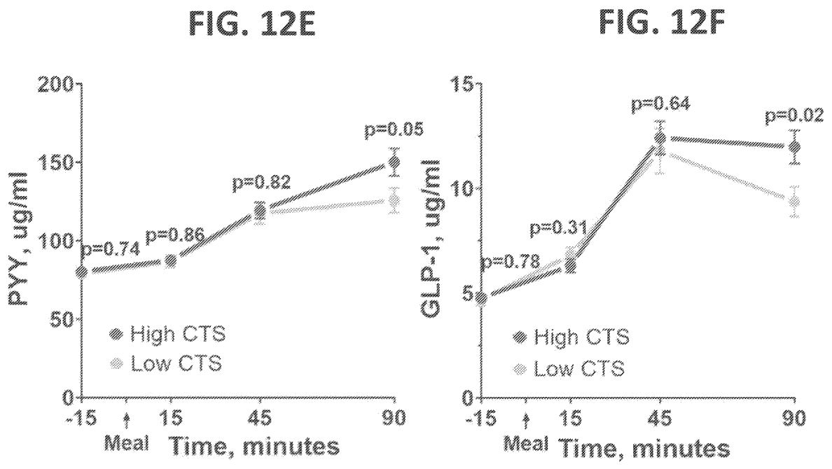

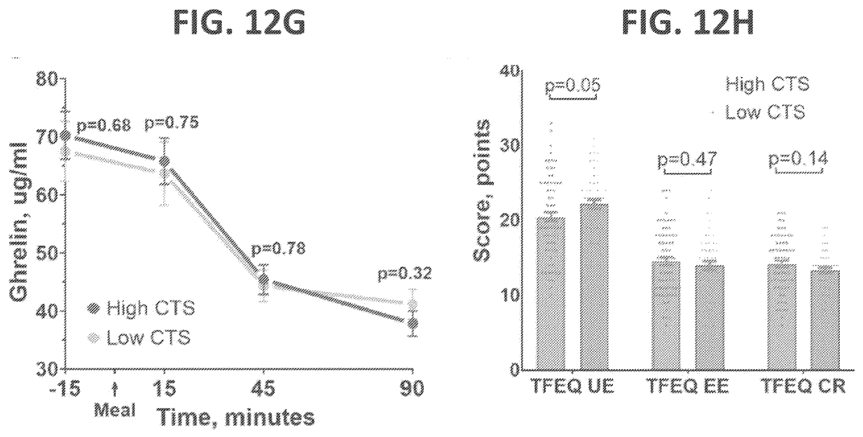

Given the observed differences between sex categories (FIG. 11C) the participant cohort was stratified into quartiles based on their satiation responses, distinguishing between males and females. Those falling within quartile 1, characterized by satiation responses of <650 kcal for females and <927 kcal for males, were hereafter referred to as low CTS. Conversely, participants in quartile 4, exhibiting satiation responses of >977 kcal for females and >1374 kcal for males, are referred to as high CTS (Table 6 and FIG. 12A). Participants positioned in quartiles 2 and 3 were excluded from these analyses.

Females in the high CTS group had a faster solid gastric emptying half-time (115.4±28.2 min) than the low CTS group (129±31.2 min; p<0.001), difference not seen in males (100.6±31.6 min vs. 110.5±32.9 min; p=0.15) (FIG. 12B). After the standardized breakfast, participants with high CTS reported more hunger and lower fullness across all measured time points, except for fullness sensation at 30 minutes post-breakfast, when compared to participants with low CTS (FIGS. 11C-D). In addition, participants with high CTS exhibited elevated levels of GLP-1 (11.98±8.5 μg/ml vs. 9.36±6.9 μg/ml, p=0.02) and numerically higher PYY (150.10±79.56 μg/ml vs. 125.82±59.97 μg/ml, p=0.05) 90 minutes after breakfast, with no significant changes in ghrelin secretion (FIG. 11E-11G). There were no significant differences in validated questionnaires for TFEQ among groups (FIG. 11H). These findings emphasize the significant interpersonal variability of satiation and reveal that individuals with a high CTS are a distinct group with unique subjective and objective responses to the same meal, further emphasizing the complexity of satiation regulation.

### Association of Genetic Variations and Satiation

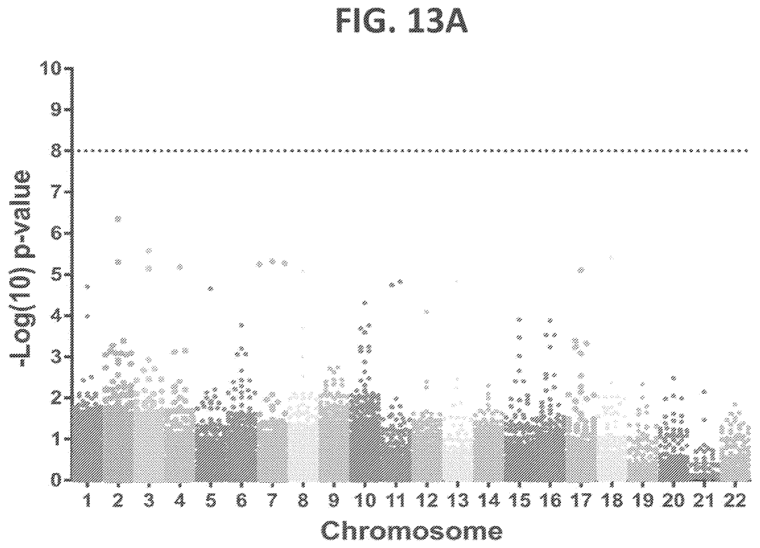

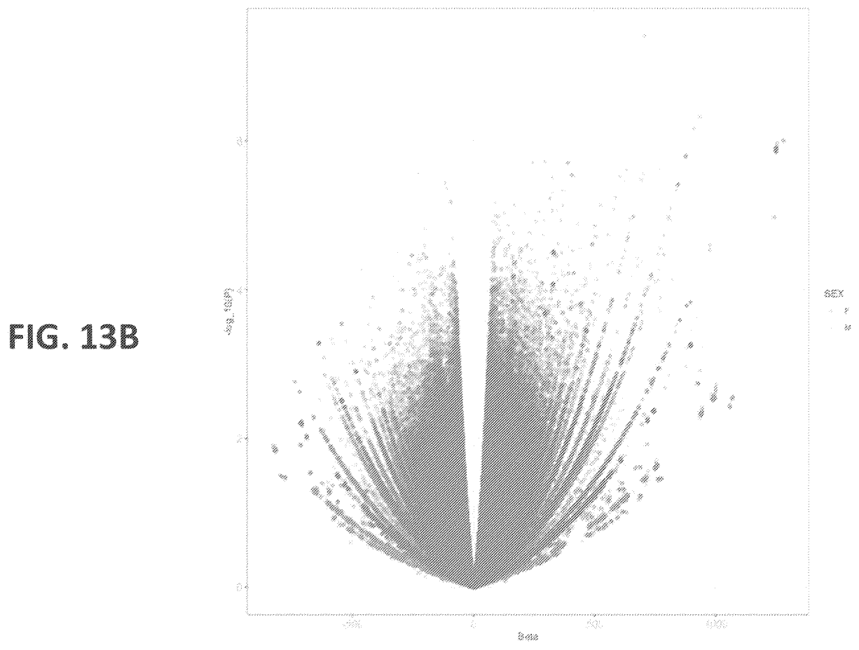

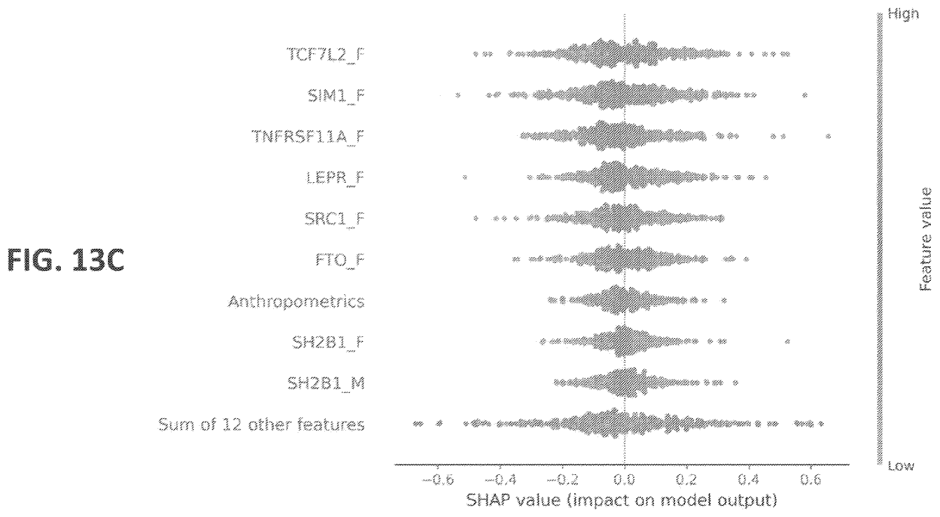

Despite the study's modest sample size of 717 participants, a subset of 497 individuals with available DNA data passed the quality control for the genome-wide association study (GWAS). While acknowledging the discrepancy in scale compared to extensive cohorts in landmark obesity-related GWAS studies, which included up to 300,000 individuals¹³⁻¹⁵, the focus of this study was on exploring potential genetic signals associated with CTS (FIG. 13A). However, in this study's cohort, the GWAS results did not reveal any genes significantly linked to CTS in participants with obesity. This limitation underscores the challenges of detecting subtle genetic signals in a comparatively smaller cohort and emphasizes the need for larger-scale investigations in the future (FIG. 13A). A volcano plot portraying the association between different genetic features and CTS among male and female subjects is presented in (FIG. 13B).

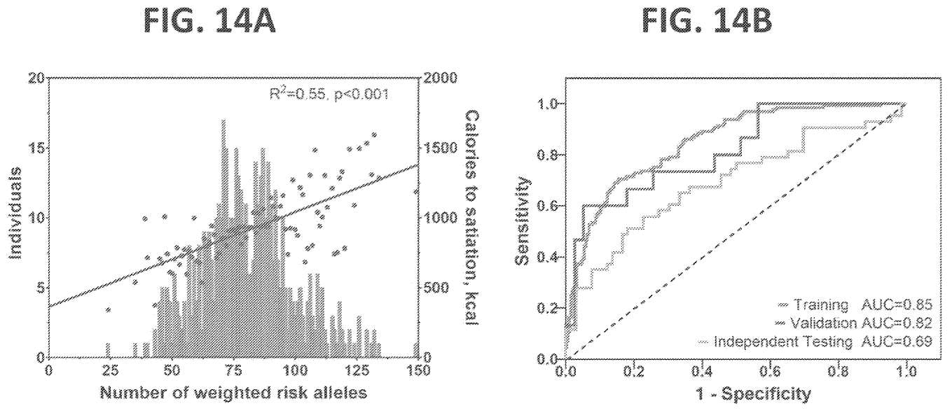

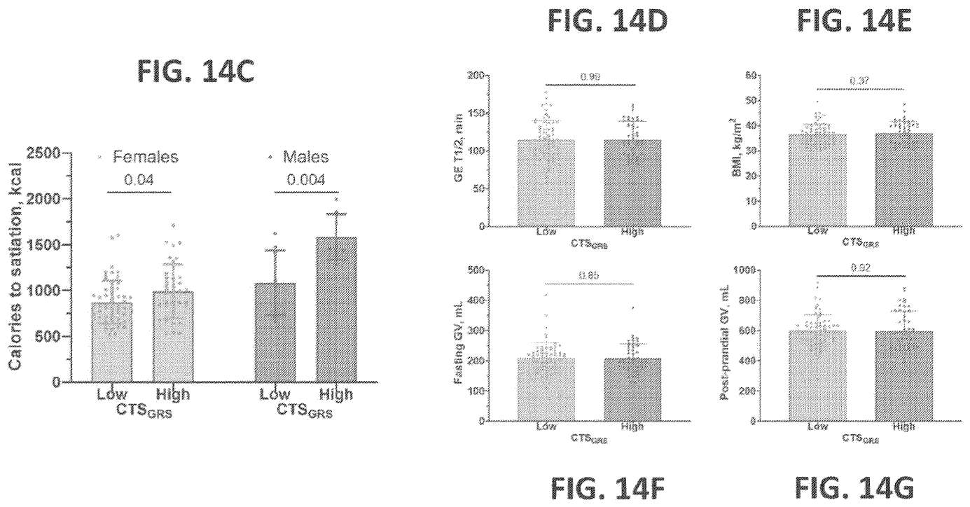

Consequently, a candidate gene approach¹⁶ was pursued, focusing on genes associated with appetite regulation, energy expenditure, lipid metabolism, and adipogenesis identified in prior genetic studies. This targeted exploration yielded a curated set of 40 genes and 19,161 unique annotated variants (Table 5). Utilizing weighted risk alleles associated with CTS from the GWAS, in conjunction with relevant demographic information, the top 10% most correlated single nucleotide polymorphisms (SNPs) were identified. These SNPs were derived from SNPs located within the 40 genes selected during the literature-review and that showed a genetic variation across samples. The SNPs that had a Spearman correlation between the number of effect alleles and CTS across samples >0.06 were selected, which resulted in 227 informative SNPs (Table 11). Subsequently, a polygenic risk score (PRS) for CTS was developed by summing the weighted risk alleles of the selected SNPs. The PRS demonstrated a robust correlation with CTS(R²=0.55; p<0.001; FIG. 14A).

TABLE 11
List of Single Nucleotide Polymorphisms (SNP) used
for the development of the polygenic risk score.

| | | Spearman | | |
|---|---|---|---|---|
| Number | SNP | Correlation | T-value | P-value |
| 1 | kgp17140923 | 0.167727 | 3.905758 | 0.000106 |
| 2 | kgp6282984 | 0.161602 | 3.759224 | 0.00019 |
| 3 | kgp15719150 | 0.153522 | 3.566602 | 0.000395 |
| 4 | kgp2496504 | 0.145592 | 3.378276 | 0.000783 |
| 5 | kgp10708165 | 0.13185 | 3.053468 | 0.002376 |
| 6 | rs7186637 | 0.13173 | 3.050649 | 0.002398 |
| 7 | kgp18047302 | 0.131427 | 3.043508 | 0.002455 |
| 8 | rs9811546 | 0.129501 | 2.998133 | 0.002845 |
| 9 | kgp4442651 | 0.129446 | 2.99683 | 0.002857 |
| 10 | kgp2375678 | 0.129032 | 2.987093 | 0.002948 |
| 11 | kgp6124457 | 0.127011 | 2.939538 | 0.003431 |
| 12 | kgp5650467 | 0.125371 | 2.900956 | 0.003876 |
| 13 | exm-rs3817334 | 0.125066 | 2.893792 | 0.003964 |
| 14 | rs16953089 | 0.118577 | 2.741448 | 0.006325 |
| 15 | rs16952608 | 0.115806 | 2.676503 | 0.007671 |
| 16 | kgp7628825 | 0.115031 | 2.658344 | 0.008091 |
| 17 | kgp692638 | 0.114593 | 2.648092 | 0.008337 |
| 18 | rs7598617 | 0.113752 | 2.628417 | 0.008829 |
| 19 | rs6708615 | 0.113341 | 2.618783 | 0.009078 |
| 20 | kgp4853206 | 0.112258 | 2.593441 | 0.009766 |
| 21 | kgp2989978 | 0.107702 | 2.486915 | 0.013194 |
| 22 | kgp2698156 | 0.107496 | 2.482123 | 0.013371 |
| 23 | kgp8569918 | 0.106749 | 2.464657 | 0.014032 |
| 24 | rs953567 | 0.106505 | 2.458961 | 0.014254 |
| 25 | kgp10318111 | 0.106362 | 2.455627 | 0.014386 |
| 26 | kgp9879049 | 0.105697 | 2.440103 | 0.015012 |
| 27 | kgp7337450 | 0.105207 | 2.428668 | 0.015488 |
| 28 | rs9870384 | 0.103619 | 2.391608 | 0.017124 |
| 29 | rs1105985 | 0.103369 | 2.385763 | 0.017396 |
| 30 | rs1822300 | 0.103165 | 2.381006 | 0.01762 |
| 31 | kgp16450975 | 0.101863 | 2.350636 | 0.019109 |
| 32 | kgp16358165 | 0.101863 | 2.350636 | 0.019109 |
| 33 | kgp16275485 | 0.101863 | 2.350636 | 0.019109 |
| 34 | kgp16433458 | 0.101863 | 2.350636 | 0.019109 |
| 35 | kgp16405397 | 0.101863 | 2.350636 | 0.019109 |
| 36 | kgp4221755 | 0.101863 | 2.350636 | 0.019109 |
| 37 | kgp16477195 | 0.101863 | 2.350636 | 0.019109 |
| 38 | kgp4735166 | 0.101774 | 2.348572 | 0.019214 |
| 39 | rs818215 | 0.101007 | 2.330687 | 0.020146 |
| 40 | rs17091269 | 0.099938 | 2.305768 | 0.021511 |
| 41 | kgp1418098 | 0.099693 | 2.300056 | 0.021835 |
| 42 | kgp8510591 | 0.099359 | 2.292278 | 0.022283 |
| 43 | rs10838738 | 0.098462 | 2.271371 | 0.023527 |
| 44 | rs13334933 | 0.098437 | 2.270801 | 0.023562 |
| 45 | kgp11428873 | 0.098125 | 2.263527 | 0.024009 |
| 46 | rs9936768 | 0.098116 | 2.26331 | 0.024023 |
| 47 | kgp2615591 | 0.097459 | 2.248023 | 0.024987 |
| 48 | rs10305432 | 0.096607 | 2.228182 | 0.02629 |
| 49 | kgp11789386 | 0.094991 | 2.190555 | 0.028921 |
| 50 | kgp10699695 | 0.092869 | 2.141206 | 0.032715 |
| 51 | kgp8097781 | 0.092849 | 2.140742 | 0.032753 |
| 52 | kgp6969125 | 0.092642 | 2.135919 | 0.033146 |
| 53 | kgp6008222 | 0.092423 | 2.130833 | 0.033565 |
| 54 | kgp8209301 | 0.091954 | 2.119913 | 0.03448 |
| 55 | kgp3279741 | 0.09192 | 2.11912 | 0.034547 |
| 56 | kgp11054248 | 0.091699 | 2.113999 | 0.034984 |
| 57 | rs6667659 | 0.090809 | 2.093314 | 0.036799 |
| 58 | kgp590141 | 0.090569 | 2.087719 | 0.037303 |
| 59 | rs10490895 | 0.090253 | 2.080372 | 0.037974 |
| 60 | kgp6569390 | 0.089927 | 2.072807 | 0.038676 |
| 61 | kgp15504852 | 0.089762 | 2.068964 | 0.039036 |
| 62 | kgp235847 | 0.089292 | 2.05805 | 0.040077 |
| 63 | kgp2343306 | 0.089041 | 2.05222 | 0.040642 |
| 64 | kgp8443782 | 0.087881 | 2.025275 | 0.043343 |
| 65 | rs4301023 | 0.087361 | 2.013209 | 0.044601 |
| 66 | kgp432157 | 0.086693 | 1.997692 | 0.046264 |
| 67 | kgp6294121 | 0.086045 | 1.982636 | 0.047927 |
| 68 | kgp9138399 | 0.085955 | 1.980544 | 0.048162 |
| 69 | kgp9715819 | 0.085618 | 1.972732 | 0.049048 |
| 70 | rs3845345 | 0.085618 | 1.972732 | 0.049048 |
| 71 | rs6604871 | 0.085618 | 1.972732 | 0.049048 |
| 72 | kgp918084 | 0.085498 | 1.96994 | 0.049368 |
| 73 | kgp4634414 | 0.08527 | 1.964646 | 0.04998 |
| 74 | rs7527501 | 0.084596 | 1.949002 | 0.051825 |
| 75 | s3856029 | 0.084596 | 1.949002 | 0.051825 |
| 76 | kgp9947320 | 0.084588 | 1.94883 | 0.051846 |
| 77 | rs720090 | 0.084549 | 1.947931 | 0.051954 |
| 78 | s977145 | 0.083553 | 1.92481 | 0.054791 |
| 79 | rs12135145 | 0.083553 | 1.92481 | 0.054791 |
| 80 | kgp1068110 | 0.083553 | 1.92481 | 0.054791 |
| 81 | kgp7284958 | 0.083376 | 1.920705 | 0.055308 |
| 82 | kgp2950350 | 0.083376 | 1.920705 | 0.055308 |
| 83 | rs16953002 | 0.083187 | 1.916315 | 0.055866 |
| 84 | rs290481 | 0.082742 | 1.906004 | 0.057193 |
| 85 | rs3828137 | 0.082393 | 1.897916 | 0.058253 |
| 86 | rs637871 | 0.082274 | 1.895154 | 0.058619 |
| 87 | rs11125662 | 0.082226 | 1.894037 | 0.058767 |
| 88 | kgp3446380 | 0.081984 | 1.888432 | 0.059517 |
| 89 | rs17224310 | 0.081984 | 1.888432 | 0.059517 |
| 90 | kgp5392217 | 0.081984 | 1.888432 | 0.059517 |
| 91 | kgp2383281 | 0.081984 | 1.888432 | 0.059517 |
| 92 | kgp782640 | 0.081466 | 1.876415 | 0.061151 |
| 93 | kgp21635081 | 0.081369 | 1.874171 | 0.06146 |
| 94 | rs12032861 | 0.081202 | 1.870282 | 0.061999 |
| 95 | rs1872552 | 0.080926 | 1.863893 | 0.062893 |
| 96 | kgp8125391 | 0.080392 | 1.851503 | 0.064656 |
| 97 | kgp409747 | 0.080325 | 1.849964 | 0.064878 |
| 98 | rs1486087 | 0.080217 | 1.847453 | 0.065242 |
| 99 | rs836964 | 0.080151 | 1.845921 | 0.065464 |
| 100 | rs9436297 | 0.0799 | 1.84011 | 0.066314 |
| 101 | kgp6402441 | 0.079309 | 1.826419 | 0.068353 |
| 102 | rs1545255 | 0.078718 | 1.812703 | 0.070447 |
| 103 | kgp12241790 | 0.078614 | 1.810314 | 0.070816 |
| 104 | rs35681 | 0.078364 | 1.804514 | 0.071722 |
| 105 | kgp2042396 | 0.078283 | 1.802625 | 0.072018 |
| 106 | rs3826169 | 0.078283 | 1.802625 | 0.072018 |
| 107 | rs3102916 | 0.077925 | 1.794345 | 0.073331 |
| 108 | kgp6072312 | 0.077806 | 1.791583 | 0.073773 |
| 109 | kgp10784457 | 0.077806 | 1.791583 | 0.073773 |
| 110 | kgp5527704 | 0.077335 | 1.780681 | 0.07554 |
| 111 | kgp12498401 | 0.077335 | 1.780681 | 0.07554 |
| 112 | kgp892392 | 0.077333 | 1.780616 | 0.075551 |
| 113 | kgp10812923 | 0.076865 | 1.769797 | 0.077339 |
| 114 | rs10493544 | 0.076807 | 1.768452 | 0.077564 |
| 115 | exm-rs10767664 | 0.076371 | 1.758355 | 0.079268 |
| 116 | kgp8874091 | 0.076267 | 1.755928 | 0.079682 |
| 117 | kgp2457524 | 0.075821 | 1.745619 | 0.08146 |
| 118 | rs9677360 | 0.075184 | 1.730853 | 0.084063 |
| 119 | rs7103411 | 0.075176 | 1.730682 | 0.084094 |
| 120 | rs988748 | 0.075176 | 1.730682 | 0.084094 |
| 121 | kgp1616578 | 0.07475 | 1.72081 | 0.085872 |
| 122 | rs3102906 | 0.07475 | 1.72081 | 0.085872 |
| 123 | kgp11818021 | 0.074695 | 1.719535 | 0.086104 |
| 124 | kgp5070734 | 0.074692 | 1.71948 | 0.086114 |
| 125 | kgp6519946 | 0.074582 | 1.716921 | 0.086581 |
| 126 | kgp3635413 | 0.074466 | 1.714236 | 0.087073 |
| 127 | rs1003986 | 0.074419 | 1.71316 | 0.087271 |
| 128 | rs943762 | 0.07432 | 1.710868 | 0.087694 |
| 129 | kgp6895955 | 0.074133 | 1.706539 | 0.088497 |
| 130 | kgp15475437 | 0.074117 | 1.706162 | 0.088567 |
| 131 | rs5445 | 0.07411 | 1.705989 | 0.088599 |
| 132 | kgp7749359 | 0.074037 | 1.704295 | 0.088915 |
| 133 | kgp10556806 | 0.074037 | 1.704295 | 0.088915 |
| 134 | rs1591382 | 0.073567 | 1.693436 | 0.090963 |
| 135 | rs35682 | 0.073055 | 1.681585 | 0.093242 |
| 136 | rs12933996 | 0.072769 | 1.674957 | 0.094536 |
| 137 | kgp4773550 | 0.072678 | 1.672847 | 0.094951 |
| 138 | rs6233 | 0.072617 | 1.671454 | 0.095226 |
| 139 | rs11594681 | 0.072405 | 1.66655 | 0.096198 |
| 140 | rs1003985 | 0.072378 | 1.665916 | 0.096324 |
| 141 | kgp5905138 | 0.072305 | 1.664225 | 0.096662 |
| 142 | kgp54820 | 0.072283 | 1.663708 | 0.096765 |
| 143 | kgp15891030 | 0.072279 | 1.663613 | 0.096784 |
| 144 | kgp1937988 | 0.071943 | 1.655851 | 0.098347 |
| 145 | kgp5787334 | 0.071943 | 1.655851 | 0.098347 |
| 146 | kgp15622188 | 0.071943 | 1.655851 | 0.098347 |
| 147 | rs7518885 | 0.071943 | 1.655851 | 0.098347 |
| 148 | rs1514175 | 0.071368 | 1.642554 | 0.101072 |
| 149 | kgp6125022 | 0.071296 | 1.640878 | 0.101419 |
| 150 | kgp4152198 | 0.071264 | 1.64014 | 0.101573 |
| 151 | rs7616936 | 0.071122 | 1.63685 | 0.102259 |
| 152 | kgp1127580 | 0.070989 | 1.633785 | 0.102901 |
| 153 | kgp7273935 | 0.070837 | 1.63027 | 0.103642 |
| 154 | rs10305512 | 0.070549 | 1.623596 | 0.10506 |
| 155 | rs7187609 | 0.070388 | 1.619889 | 0.105854 |
| 156 | kgp10561318 | 0.070103 | 1.613285 | 0.107281 |
| 157 | kgp264437 | 0.070068 | 1.612471 | 0.107458 |
| 158 | kgp8769262 | 0.069804 | 1.606373 | 0.108791 |
| 159 | kgp12057556 | 0.069798 | 1.606227 | 0.108823 |
| 160 | rs4386132 | 0.069753 | 1.605198 | 0.109049 |
| 161 | kgp1244888 | 0.069421 | 1.597527 | 0.110748 |
| 162 | kgp665347 | 0.069309 | 1.594923 | 0.111329 |
| 163 | rs1486090 | 0.069293 | 1.594547 | 0.111413 |
| 164 | kgp5555721 | 0.069261 | 1.593805 | 0.111579 |
| 165 | kgp7894359 | 0.068987 | 1.587473 | 0.113005 |
| 166 | rs7193938 | 0.068797 | 1.583095 | 0.114 |
| 167 | rs9925908 | 0.068797 | 1.583095 | 0.114 |
| 168 | rs9871198 | 0.068667 | 1.580091 | 0.114686 |
| 169 | kgp3395086 | 0.068574 | 1.577937 | 0.11518 |
| 170 | rs11030104 | 0.068568 | 1.577796 | 0.115212 |
| 171 | kgp2818301 | 0.068294 | 1.57145 | 0.116678 |
| 172 | kgp10978885 | 0.068269 | 1.570883 | 0.11681 |
| 173 | rs9861237 | 0.067972 | 1.564011 | 0.118415 |
| 174 | kgp7545237 | 0.067914 | 1.562675 | 0.118729 |
| 175 | kgp2849902 | 0.067907 | 1.56251 | 0.118768 |
| 176 | kgp8038575 | 0.067834 | 1.560816 | 0.119167 |
| 177 | rs842192 | 0.067799 | 1.560017 | 0.119356 |
| 178 | rs9924877 | 0.067741 | 1.558683 | 0.119672 |
| 179 | rs290487 | 0.067684 | 1.557364 | 0.119984 |
| 180 | kgp7666751 | 0.067541 | 1.554057 | 0.120771 |
| 181 | rs9822731 | 0.067169 | 1.545466 | 0.122834 |
| 182 | kgp10829441 | 0.066626 | 1.532899 | 0.125901 |
| 183 | rs7178761 | 0.066613 | 1.532602 | 0.125974 |
| 184 | rs4078063 | 0.066613 | 1.532602 | 0.125974 |
| 185 | rs708258 | 0.066543 | 1.530976 | 0.126375 |
| 186 | rs17022413 | 0.066442 | 1.528645 | 0.126952 |
| 187 | kgp18204408 | 0.066442 | 1.528645 | 0.126952 |
| 188 | kgp313427 | 0.066115 | 1.521101 | 0.128834 |
| 189 | kgp1355091 | 0.065468 | 1.506152 | 0.132627 |
| 190 | rs6585196 | 0.065161 | 1.499043 | 0.134461 |
| 191 | kgp22835587 | 0.06511 | 1.497865 | 0.134767 |
| 192 | rs2143734 | 0.064871 | 1.492345 | 0.136207 |
| 193 | rs2303175 | 0.064856 | 1.492013 | 0.136294 |
| 194 | rs3732136 | 0.064758 | 1.489751 | 0.136888 |
| 195 | kgp17476155 | 0.064621 | 1.486585 | 0.137722 |
| 196 | kgp17176845 | 0.064621 | 1.486585 | 0.137722 |
| 197 | kgp1944871 | 0.06421 | 1.477094 | 0.140248 |
| 198 | rs1044677 | 0.06413 | 1.475234 | 0.140747 |
| 199 | kgp7996450 | 0.064036 | 1.473072 | 0.141329 |
| 200 | rs17729386 | 0.063748 | 1.46641 | 0.143133 |
| 201 | kgp3664637 | 0.063413 | 1.458673 | 0.145251 |
| 202 | rs35683 | 0.06341 | 1.458617 | 0.145266 |
| 203 | rs17022384 | 0.063168 | 1.453013 | 0.146815 |
| 204 | kgp11528471 | 0.063105 | 1.451564 | 0.147218 |
| 205 | kgp4280672 | 0.063021 | 1.449611 | 0.147762 |
| 206 | rs11600974 | 0.063021 | 1.449611 | 0.147762 |
| 207 | kgp18059549 | 0.063006 | 1.449281 | 0.147854 |
| 208 | rs1861868 | 0.06282 | 1.444982 | 0.149057 |
| 209 | kgp15546318 | 0.062231 | 1.431373 | 0.152916 |
| 210 | kgp12563751 | 0.061977 | 1.425515 | 0.154601 |
| 211 | kgp5766551 | 0.061897 | 1.42367 | 0.155134 |
| 212 | rs4640343 | 0.061828 | 1.422068 | 0.155598 |
| 213 | rs9309989 | 0.06176 | 1.420494 | 0.156055 |
| 214 | kgp370158 | 0.06176 | 1.420494 | 0.156055 |
| 215 | kgp173451 | 0.061571 | 1.416148 | 0.157323 |
| 216 | rs7612090 | 0.061242 | 1.408547 | 0.159559 |
| 217 | kgp3890866 | 0.061234 | 1.408365 | 0.159613 |
| 218 | kgp9026044 | 0.061038 | 1.403839 | 0.160956 |
| 219 | rs357200 | 0.061038 | 1.403839 | 0.160956 |
| 220 | kgp914141 | 0.06083 | 1.399039 | 0.16239 |
| 221 | kgp15656986 | 0.060777 | 1.397803 | 0.16276 |
| 222 | kgp15393521 | 0.060777 | 1.397803 | 0.16276 |
| 223 | rs9988578 | 0.060777 | 1.397803 | 0.16276 |
| 224 | rs11209939 | 0.060777 | 1.397803 | 0.16276 |
| 225 | kgp4025272 | 0.060704 | 1.39613 | 0.163263 |
| 226 | rs2665272 | 0.06058 | 1.393263 | 0.164128 |
| 227 | rs12030511 | 0.060372 | 1.388458 | 0.165584 |

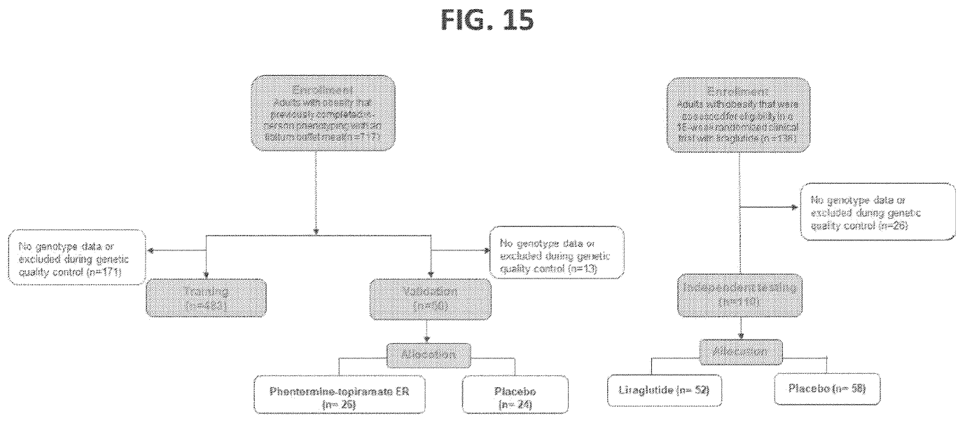

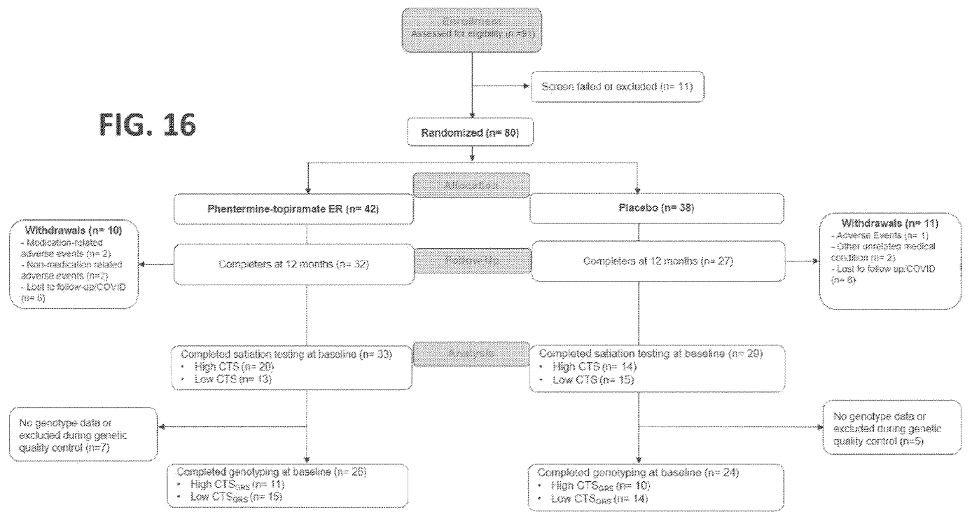

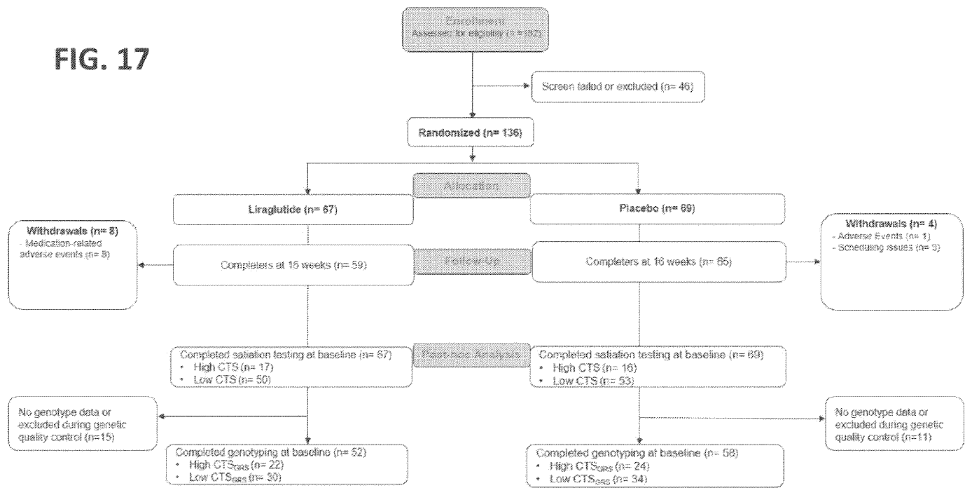

As a next step, machine-learning techniques were employed to create a machine-learning-assisted gene risk score (ML-GRS) aimed at predicting high CTS corresponding to quartile 1, while quartiles 2 to 4 represented low CTS (FIG. 10C). This method, known to improve the predictive power of PRSs by accounting for non-linear genetic effects^{17,18}, utilized demographic, anthropometric parameters, and genomic data from our participants with available genetic information. Using forward feature selection and random forest predictors, 16 genetic risk scores associated with 11 genes were identified, including SIM1, PCSK1, SH2B1, LEPR, UCP2, FTO, TCF7L2, GLP1R, TNFRSF11A, and ADRA2A (FIG. 13C). The dataset was divided into training (483 participants) and validation cohort (n=50) (Table 12A, 12B and 13 and FIG. 15). This validation cohort where those who participated in the 52-week, double-blind, placebo-controlled, randomized clinical trial comparing phentermine-topiramate extended-release versus placebo and completed phenotype testing (N=62), with available genetic data (n=50) (Tables 14 and 15 and FIG. 16). Furthermore, an independent testing cohort, comprising participants from a 16-week, double-blind, liraglutide vs placebo-controlled, randomized clinical trial (n=136) who had available genetic data (n=110), was used for additional validation (Table 16 and 17 and FIG. 17). The final ML-GRS model to predict CTS (CTS_{GRS}) had a training area under the curve (AUC) of 0.85 (95% CI: 0.81 to 0.89), with a mean cross-validated AUC of 0.82 (95% CI: 0.67 to 0.95) across the 10-fold validation. In the validation cohort, the CTS_{GRS} achieved an AUC of 0.82 (95% CI: 0.69 to 0.94), while in the independent testing cohort, it attained an AUC of 0.69 (95% CI: 0.59 to 0.80) (FIG. 14B). Table 12A-B. Number of samples with high or low CTS_{GRS} predictions categorized by actual high or low CTS in the A) validation and B) independent testing cohorts.

| | Actual CTS | | |
|---|---|---|---|
| Predicted | High | Low | Total |

A: Validation Cohort

| | | | |
|---|---|---|---|
| High | 17 | 4 | 21 |
| Low | 12 | 17 | 29 |
| Total | 29 | 21 | 50 |

B: Independent Testing Cohort

| | | | |
|---|---|---|---|
| High | 18 | 28 | 46 |
| Low | 11 | 53 | 64 |
| Total | 29 | 81 | 110 |

TABLE 13
Demographic characteristics and parameters of energy balance in the
training cohort, validation cohort and independent testing cohort.

| | | | Independent |
|---|---|---|---|
| | Training cohort | Validation cohort | testing cohort |
| | n = 483 | n = 50 | n = 110 |
| Age, years | 41.2 ± 11.2 | 41.2 ± 10.0 | 38.6 ± 10.8 |
| Female, n(%) | 359 (74.3) | 43 (86.0) | 93 (84.5) |
| Weight, kg | 106.3 ± 23.4 | 109.6 ± 26.6 | 103.4 ± 14.3 |
| Height, cm | 169.2 ± 9.0 | 168.6 ± 9.2 | 167.8 ± 7.9 |
| Harris-Benedict, kcal/day | 1889.9 ± 357.0 | 1809.5 ± 360.6 | 1895.3 ± 189.4 |
| BMI, kg/m2 | 37.0 ± 7.4 | 38.2 ± 6.9 | 36.7 ± 4.3 |
| Gastric emptying 120 min, ratio | 0.53 ± 0.17 | 0.47 ± 0.16 | 0.55 ± 0.14 |
| Gastric emptying T 1/2, min | 127.4 ± 34.0 | 127.9 ± 30.2 | 114.9 ± 25.2 |
| Calories to satiation, kcal | 927.5 ± 318.8 | 929.1 ± 314.9 | 980.7 ± 321.9 |
| Fat Mass, % | 48.4 ± 6.6 | 50.8 ± 4.7 | 47.0 ± 6.0 |

Abbreviations used: BMI, body mass index.

TABLE 14
Demographic characteristics and parameters of energy balance in
the validation cohort for participants that underwent ad libitum
meal testing for the 12-month, placebo-controlled, randomized
clinical trial with phentermine-topiramate extended-release.

| | Phentermine - | |
|---|---|---|
| | Topiramate | Placebo |
| | n= 33 | n = 29 |
| Age, years | 41.8 ± 9.6 | 41.0 ± 10.2 |
| Females, n (%) | 28 (84.8) | 25 (86.2) |
| Weight, kg | 108.9 ± 17.7 | 108.4 ± 19.3 |
| Height, cm | 167.9 ± 7.5 | 169.0 ± 9.8 |
| Harris-Benedict, kcal/day | 1787.5 ± 250.4 | 1796.4 ± 299.1 |
| BMI, kg/m2 | 38.2 ± 5.3 | 37.9 ± 5.3 |
| Systolic blood pressure, | 133.8 ± 16.3 | 130.4 ± 11.4 |
| mmHg | | |
| Diastolic blood pressure, | 82.7 ± 11.1 | 79.5 ± 10.2 |
| mmHg | | |
| Pulse, bpm | 74.2 ± 9.7 | 76.8 ± 10.9 |
| Waist circumference, cm | 115.3 ± 13.3 | 113.8 ± 9.1 |
| Hip circumference, cm | 126.8 ± 10.5 | 125.8 ± 11.4 |
| Waist/hip, ratio | 0.9 ± 0.1 | 0.9 ± 0.1 |
| Gastric emptying 120 min, | 0.5 ± 0.1 | 0.5 ± 0.2 |
| ratio | | |
| Gastric emptying T 1/2, min | 119.7 ± 29.0 | 136.5 ± 31.3 |
| Calories to satiation, kcal | 918.4 ± 297.9 | 907.8 ± 301.3 |
| VAS-hunger 2 hours CTS, | 25.2 ± 18.9 | 30.3 ± 22.2 |
| mm | | |
| VAS-Fullness 2 hours CTS, | 69.4 ± 17.9 | 63.2 ± 19.4 |
| mm | | |
| VAS-satisfaction 2 hours | 70.0 ± 19.5 | 61.0 ± 22.1 |
| CTS, mm | | |
| Total Lean Mass, kg | 53.6 ± 9.3 | 54.2 ± 10.6 |
| Fat Mass, % | 48.5 ± 6.5 | 47.7 ± 4.5 |
| Weight observation at 3 | 29 (87.9) | 27 (93.1) |
| months, % | | |
| Weight observation at 6 | 27 (81.8) | 25 (86.2) |
| months, % | | |
| Weight observation at 9 | 24 (72.7) | 23 (79.3) |
| months, % | | |
| Weight observation at 12 | 23 (69.7) | 23 (79.3) |
| months, % | | |

Abbreviations include: BMI, body mass index; Calories to satiation, CTS; Visual Analogue Scale, VAS.

TABLE 15
Demographic characteristics and parameters of energy balance
in the validation cohort for participants that underwent genotyping
for the 12-month, placebo-controlled, randomized clinical trial
with phentermine-topiramate extended-release.

| | Phentermine - | |
|---|---|---|
| | Topiramate | Placebo |
| | n = 26 | n = 24 |
| Age, years | 41.2 ± 9.8 | 41.4 ± 10.6 |
| Females, n (%) | 22 (84.6) | 21 (87.5) |
| Weight, kg | 107.5 ± 19.0 | 107.6 ± 19.8 |
| Height, cm | 167.7 ± 7.8 | 169.2 ± 10.7 |
| Harris-Benedict, kcal/day | 1773.3 ± 272.9 | 1784.7 ± 311.3 |
| BMI, kg/m2 | 37.7 ± 5.4 | 37.5 ± 5.0 |
| Systolic blood pressure, | 133.4 ± 16.7 | 130.2 ± 12.5 |
| mmHg | | |
| Diastolic blood pressure, | 81.1 ± 11.8 | 79.3 ± 10.6 |
| mmHg | | |
| Pulse, bpm | 72.6 ± 9.3 | 75.7 ± 10.3 |
| Waist circumference, cm | 112.4 ± 12.2 | 112.6 ± 9.3 |
| Hip circumference, cm | 124.8 ± 9.5 | 125.2 ± 11.6 |
| Waist/hip, ratio | 0.9 ± 0.1 | 0.9 ± 0.1 |
| Gastric emptying 120 min, | 0.5 ± 0.2 | 0.5 ± 0.2 |
| ratio | | |
| Gastric emptying T 1/2, min | 121.9 ± 29.8 | 133.4 ± 32.2 |
| Calories to satiation, kcal | 964.3 ± 311.1 | 903.9 ± 322.7 |
| VAS-hunger 2 hours CTS, | 27.7 ± 19.4 | 33.6 ± 21.9 |
| mm | | |
| VAS-Fullness 2 hours CTS, | 67.0 ± 18.0 | 64.9 ± 20.1 |
| mm | | |
| VAS-satisfaction 2 hours | 67.8 ± 20.0 | 62.3 ± 20.1 |
| CTS, mm | | |
| Total Lean Mass, kg | 53.2 ± 9.9 | 54.1 ± 11.1 |
| Fat Mass, % | 48.1 ± 6.5 | 47.2 ± 4.7 |
| Weight observation at 3 | 23 (88.5) | 22 (91.7) |
| months, % | | |
| Weight observation at 6 | 21 (80.8) | 21 (87.5) |
| months, % | | |
| Weight observation at 9 | 20 (76.9) | 20 (83.3) |
| months, % | | |
| Weight observation at 12 | 19 (73.1) | 20 (83.3) |
| months, % | | |

Abbreviations include: BMI, body mass index; Calories to satiation, CTS; Visual Analogue Scale, VAS.

TABLE 16
Demographic characteristics and parameters of energy balance
in the independent testing cohort for participants that
underwent phenotyping for the 16-week, placebo-controlled,
randomized clinical trial with liraglutide.

| | Liraglutide | Placebo |
|---|---|---|
| | n = 67 | n = 69 |
| Age, years | 40.1 ± 10.6 | 37.8 ± 10.6 |
| Female, n (%) | 59 (88.1) | 59 (85.5) |
| Weight, kg | 101.8 ± 14.3 | 103.2 ± 14.5 |
| Height, cm | 166.7 ± 7.7 | 168.2 ± 7.6 |
| Harris Benedict, kcal/day | 1864.1 ± 183.4 | 1899.2 ± 189.3 |
| BMI, kg/m2 | 36.6 ± 4.5 | 36.4 ± 4.1 |
| Gastric emptying 120 min, | 0.5 ± 0.1 | 0.6 ± 0.1 |
| ratio | | |
| Gastric emptying T 1/2, min | 121.2 ± 25.1 | 110.9 ± 26.6 |
| Calories to satiation, kcal | 885.4 ± 338.4 | 967.1 ± 345.9 |
| Fasting gastric volume, mL | 203.8 ± 49.4 | 209.3 ± 50.8 |
| Postprandial gastric volume, | 590.6 ± 118.6 | 601.4 ± 123.3 |
| mL | | |
| Fat Mass, % | 47.8 ± 5.2 | 46.6 ± 6.8 |
| Weight observations at 5 | 65 (97.0) | 64 (92.8) |
| wecks, % | | |
| Weight observations at 16 | 60 (89.6) | 61 (88.4) |
| weeks, % | | |

Abbreviations include: BMI, body mass index; Calories to satiation, CTS.

TABLE 17
Demographic characteristics and parameters of energy
balance in the validation cohort for participants that
underwent genotyping for the 12-month, placebo-controlled,
randomized clinical trial with liraglutide.

| | Liraglutide | Placebo |
|---|---|---|
| | n = 52 | n = 58 |
| Age, years | 39.5 ± 10.9 | 37.7 ± 10.8 |
| Female, n (%) | 45 (86.5) | 48 (82.8) |
| Weight, kg | 103.1 ± 14.2 | 103.8 ± 14.4 |
| Height, cm | 167.1 ± 7.7 | 168.5 ± 8.0 |
| Harris Benedict, kcal/day | 1882.4 ± 184.7 | 1906.9 ± 194.4 |
| BMI, kg/m2 | 36.9 ± 4.5 | 36.5 ± 4.0 |
| Gastric emptying 120 min, | 0.5 ± 0.1 | 0.6 ± 0.2 |
| ratio | | |
| Gastric emptying T 1/2, min | 121.0 23.2 | 109.5 ± 26.0 |
| Calories to satiation, kcal | 940.2 ± 304.4 | 1017.0 ± 335.3 |
| Fasting gastric volume, mL | 208.7 ± 50.1 | 209.6 ± 50.1 |
| Postprandial gastric volume, | 597.0 ± 118.5 | 598.1 ± 120.4 |
| mL | | |
| Fat Mass, % | 47.6 ± 4.8 | 46.4 ± 6.8 |
| Weight observations at 5 | 51 (98.0) | 54 (93.1) |
| weeks, % | | |
| Weight observations at 16 | 47 (90.4) | 51 (87.9) |
| weeks, % | | |

Abbreviations include: BMI, body mass index; Calories to satiation, CTS.

Participants in the independent testing cohort completed the deep phenotype testing day and additionally also completed testing for gastric volume and accommodation measured by noninvasive single photon emission computed tomography at baseline⁴. The performance of the CTS_{GRS} was tested in the independent testing cohort and it was found that individuals with a high CTS_{GRS} consumed more calories to satiation during the ad libitum meal in both females (988.9±293.7 vs. 870.7±236.0; p=0.04) and males (1582.5±239.9 vs. 1083.9±353.8; p=0.004) (FIG. 14C). It is worth noting that there were no statistically significant differences in BMI, gastric emptying for solids, and fasting and postprandial gastric volumes between high and low CTS_{GRS} prediction groups in the independent testing cohort (FIG. 14D-14G). The results underscore the high specificity of the CTS_{GRS} model in this Example for satiation measurements, as there were no differences in anthropometrics, including BMI and other components that regulate food intake such as gastric volume, or emptying.

### Satiation Surrogate-Biomarker and Weight Loss Interventions

To assess the clinical relevance of satiation, we investigated its role in response to two different weight loss interventions (FIG. 10D), phentermine-topiramate and liraglutide. Phentermine-topiramate extended-release (Qsymia®) was approved by the FDA for obesity in 2012. Phentermine suppresses appetite by releasing catecholamines in the hypothalamus, while topiramate enhances satiation and suppresses appetite through modulation of GABAergic pathways¹⁹. Phentermine-topiramate extended-release was classified as the most cost-effective medication to treat obesity²⁰. Liraglutide (Saxenda®), FDA-approved for obesity in 2014, is a GLP-1 receptor agonists that induces postprandial satiety¹⁹.

First, a prospective assessment in a 52-week, double-blind, placebo-controlled, randomized clinical trial was conducted comparing phentermine-topiramate extended-release versus placebo. Treatment allocation was concealed, and investigators were blinded for the weight loss outcomes of patients with high or low CTS and CTS_{GRS}. Then, a retrospective post-hoc analysis was conducted in a previously published 16-week, double-blind, placebo-controlled, randomized clinical trial comparing liraglutide versus placebo⁶. To study the effect of satiation and its surrogate genetic-based CTS_{GRS} biomarker, participants were included that completed the ad libitum meal, had available DNA data, and completed the trial. After stratifying participants by treatment allocation, independent t-tests were conducted at each timepoint to evaluate differences in total body weight loss across different groups.

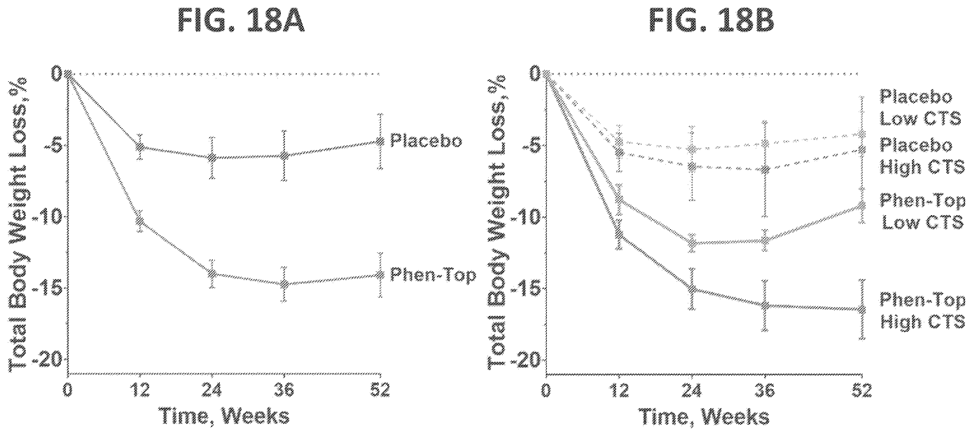

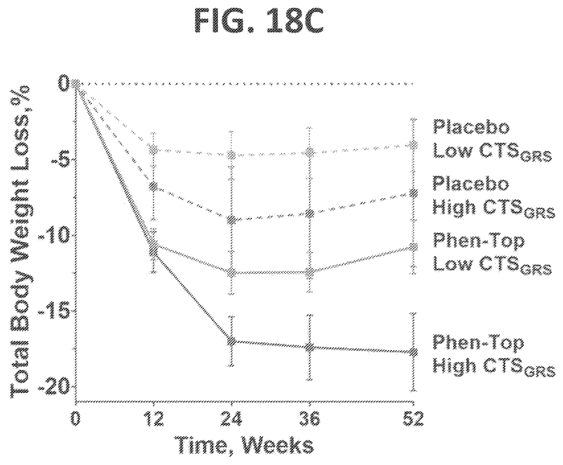

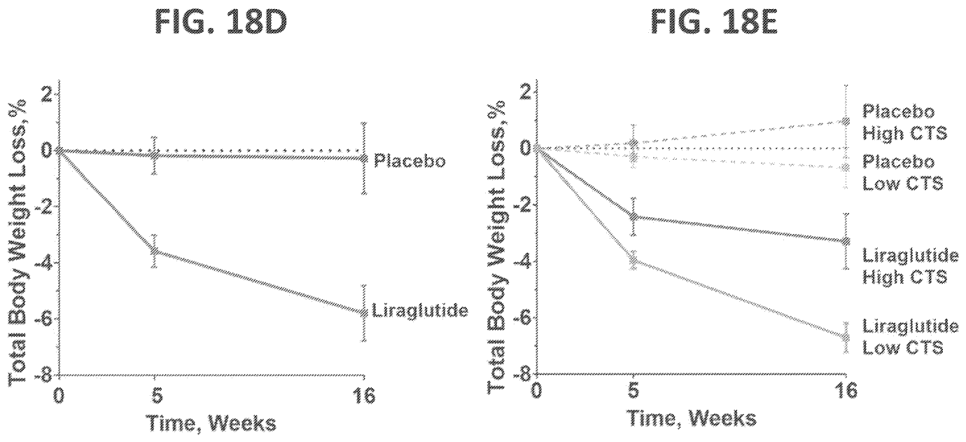

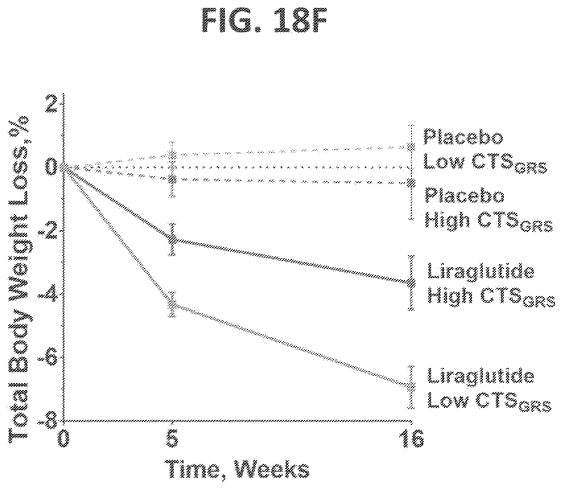

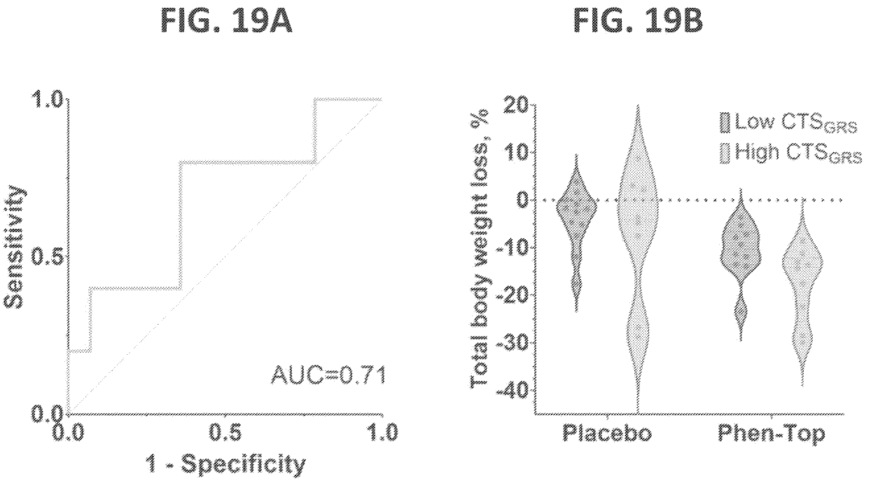

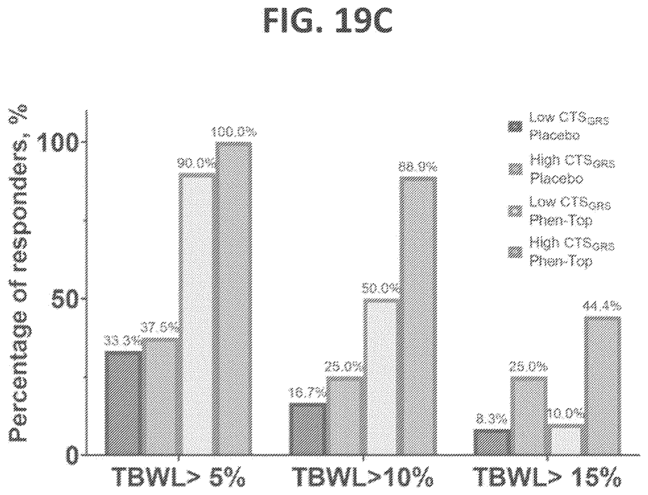

In the case of phentermine-topiramate versus placebo, a total of 62 participants that completed ad libitum meal with a mean age of 41.3±9.8 years and a mean BMI of 38.6±6.6 were included from the trial phentermine-topiramate versus placebo (FIG. 18A and Table 13). Individuals on phentermine-topiramate ER (n=23) with high CTS during the ad libitum meal achieved better weight loss outcomes compared to those with low CTS (−15.6%±7.8 vs −9.2%±3.5; p=0.03) at 52-weeks (FIG. 18B). There was no difference in weight loss outcomes at 52 weeks when comparing participants assigned to placebo with high or low CTS (p=0.78). Next, the Wilson-Brown method was used to assess the ability of predict the probability of achieving an equal to or greater than 15% total body weight loss. High CTS during the ad libitum meal predicted a probability of losing more than 15% of total weight at 52 weeks with an AUC of 0.82 (95% CI: 0.62 to 1.0; Table 7A and FIG. 19).

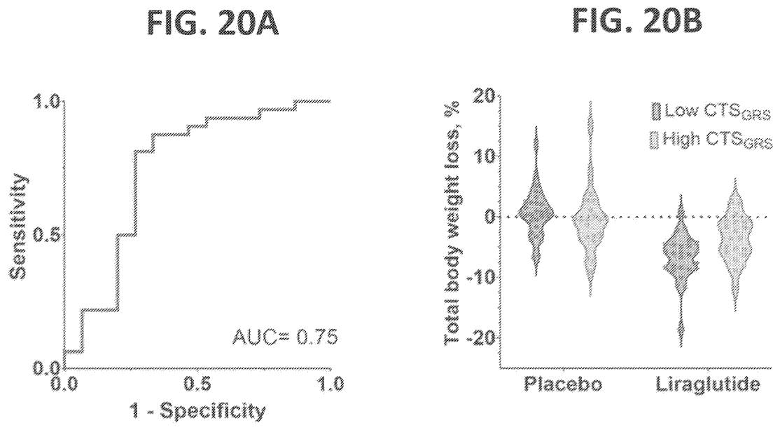

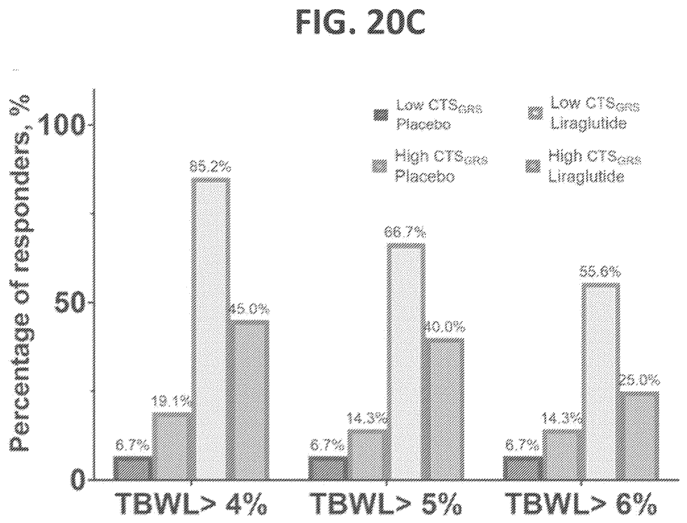

Similarly, in the cohort that underwent genetic testing, the CTS_{GRS} also proved useful in predicting the likelihood of successful weight loss in response to this intervention (Table 14). At 52-weeks, participants on phentermine-topiramate ER with a high CTS_{GRS} lost more weight than participants with a low CTS_{GRS} (−17.7±7.6 vs. −10.8%±5.6; p=0.04), while all participants assigned to placebo experienced similar weight loss outcomes at 52-weeks regardless of the CTS_{GRS} (p=0.49) (FIG. 18C). High CTS_{GRS} predicted a probability of losing more than 15% of total weight at 52 weeks with an AUC of 0.71 (95% CI: 0.42 to 0.99) (FIG. 20).

In the trial of liraglutide versus placebo trial, a total of 136 participants with a mean age of 38.9±10.6 years and a mean BMI of 36.5±4.3 were included (FIG. 18D and Table 15). Individuals with low CTS by the ad libitum meal achieved better weight loss outcomes compared to those with high CT'S at 16 weeks (−6.7%±3.4 vs −3.3%±3.9; p=0.005) (FIG. 18E). There was no difference in weight loss outcomes at 16 weeks when comparing participants assigned to placebo with high or low CTS. Low CTS by the ad libitum meal tests predicted a probability of losing more than 4% of total weight at 16 weeks with an AUC of 0.82 (95% CI: 0.62 to 1.0).

Similarly, in the cohort that underwent genetic testing (Table 16), individuals with low CTS_{GRS} had superior weight loss outcomes in response to liraglutide at 16 weeks when compared to participants with a high CTS_{GRS} (−6.9%±3.4 vs. −3.7%±3.8; p=0.004), whereas all participants assigned to placebo experienced similar weight loss outcomes at 16-weeks regardless of the CTS_{GRS} prediction (FIG. 18F). Low CTS_{GRS} tests predicted a probability of losing more than 4% of total weight at 16 weeks with an AUC of 0.75 (95% CI: 0.58 to 0.92) (Table 7B and FIG. 20). These findings underscore the potential of using satiation measurements from ad libitum meals or their corresponding genetic-based biomarker as unique predictors of the response to two specific anti-obesity medication with different mechanism of action.

### DISCUSSION

This study conducted an in-depth characterization of human satiation in a cohort of 717 participants through physiologic and behavioral testing in a tightly controlled setting, revealing significant satiation heterogeneity. The data in this Example highlighted that satiation, quantified as the calories consumed to reach fullness (i.e., CTS) during an ad libitum meal test, is influenced by a diversity of physiologic and behavioral factors. To further characterize satiation, a polygenic risk score for CTS was developed. Machine-learning techniques were then employed to develop a machine-learning gene risk score for calories to satiation (CTS_{GRS}). The clinical use of CTS and its surrogate CTS_{GRS} offer promising prospects for personalized pharmacologic interventions based on an individual's satiation characteristics as supported by the data presented with phentermine-topiramate ER and liraglutide. The findings presented herein support the clinical importance of tailored anti-obesity interventions using the key components of food intake regulation such as satiation.

The highly heterogeneous response to interventions in medicine has prompted the development of treatment strategies that are best suited to individual patients to enhance their effectiveness via precision medicine 2. The variability in response to anti-obesity interventions has been well documented in the literature over the past two decades²². Nevertheless, studies have underscored the challenges in identifying reliable predictors of weight loss response^{23,24}. For instance, these studies have identified various predictors, such as short-term weight change, psychosocial factors, and health behavior change theories, but the accuracy of these predictions has remained limited. In contrast, this study offers promising prospects for precision pharmacologic interventions considering the heterogeneity of a key regulator of food intake: satiation. Importantly, the applicability of a surrogate CTS_{GRS} biomarker was introduced in this Example to predict satiation and thereby response to anti-obesity medications.

Previous studies have used machine learning to predict food intake²⁵⁻²⁷. However, the particularity of this study lies in two facts. First, only one component of food intake regulation, satiation was focused on. Second, the current knowledge of the genetics of appetite regulation and data regarding the association between calories to satiation and specific variants was leveraged in order to develop a novel GRS. The relationship between genetics and satiation has been a subject of interest in the literature²⁸⁻³⁰. Studies exploring the complex interplay between genetic factors and satiation suggest that while genetic factors may play a role, environmental influences are also important contributors. The performance of the CTS_{GRS} presented herein is likely the reflection of this interplay.

This study extends beyond understanding the intricacies of satiation; it also explores the clinical relevance of satiation in the context of anti-obesity interventions. In a prospective assessment and in a retrospective post-hoc analysis of two randomized clinical trials, satiation measurement by calories to satiation based on the ad libitum meal and the surrogate CTS_{GRS} output predicted response to phentermine-topiramate and liraglutide. Interestingly, in individuals treated with phentermine-topiramate, high CTS in the ad libitum meal and a high CTS_{GRS} both predicted greater weight loss compared to individuals with low calories to satiation and low CTS_{GRS}. Contrasting with liraglutide, that lower CTS in the ad libitum meal and a low CTS_{GRS} predicted greater weight loss in individuals treated with liraglutide compared to individuals with high calories to satiation. The differential response observed with these two medications deserves further studies to understand their unique mechanism of action within the selected pathways; nonetheless, a potential explanation may rely on the integrity of the GLP-1 receptor to melanocortin pathway to have an appropriate response to liraglutide, a GLP-1 receptor agonist. Supporting this hypothesis is the fact that higher number of risk alleles in this pathway correlates with higher calories to satiation; as well as previous studies showing that genetic variants in the GLP-1 receptor, only one gene in the pathway, influence liraglutide response^{6,31,32}. Other key factors such as gastric function, vagal integrity, and reward pathways may be considered to explain these opposing results.

Predicting weight loss response refers to the ability to forecast how an individual will respond to a specific weight loss intervention, such as a diet, exercise program, or medical treatment³³⁻³⁵. Previous attempts at predicting weight loss response typically involved evaluating baseline characteristics, conducting health assessments, analyzing lifestyle and behavior, and considering psychological factors³⁶. However, these efforts were often limited by the high volume of data input required and the low replicability they exhibited. This study has elucidated a pathway that may predict the best responders to phentermine-topiramate or GLP-1 receptor agonist treatments through the utilization of a gene-based machine-learning algorithm. This represents a significant achievement.

In this comprehensive study, the heterogeneity of satiation is highlighted. While gender-based differences, anthropometric measures, and hormonal factors played a role, they do not fully account for this heterogeneity. Genes involved in food intake regulation were demonstrated to also partially account for this heterogeneity allowing the development of a CTS_{GRS} for predicting satiation. Furthermore, this study showcased the predictive utility of the CTS_{GRS} for individual responses to anti-obesity medications—phentermine-topiramate and liraglutide—underscoring its relevance for personalized obesity interventions.

### Summary of Example 4

Satiation is the physiologic process that regulates meal size and termination, and it is quantified by the calories consumed to reach fullness. Given its role in energy intake, changes in satiation contribute to obesity's pathogenesis. This Example employed a protocolized approach to study the components of food intake regulation including a standardized breakfast, a gastric emptying study, appetite sensation testing, and a satiation measurement by an ad libitum meal test. These studies revealed that satiation was highly variable among individuals, and while baseline characteristics, anthropometrics, body composition and hormones, contribute to this variability, these did not fully account for it. To address this gap, the role of a germline polygenic risk score was explored, which demonstrated a robust association with satiation. Furthermore, a machine-learning-assisted gene risk score was introduced to predict satiation and this prediction was leveraged to anticipate responses to anti-obesity medications. The findings underscored the significance of satiation, its inherent variability, and the potential of a genetic risk score to forecast it, ultimately allowing prediction of responses to anti-obesity interventions.

### LIST OF ABBREVIATIONS

AUC (Area Under the Curve), BMI (Body Mass Index), CTS (Calories to Satiation), ER (Extended Release), GRS (Gene Risk Score), GWAS (Genome-Wide Association Study), GLP-1 (Glucagon-Like Peptide-1), ML-GRS (Machine-Learning Assisted Gene Risk Score), PYY (Peptide YY), RCTs (Randomized Controlled Trials), ROC-AUC (Receiver Operating Characteristic Area Under the Curve). SNP (Single Nucleotide Polymorphism), TFEQ (Three-Factor Eating Questionnaire), and VAS (Visual Analog Scales).

### REFERENCES

1 World Obesity. Economic impact of overweight and obesity to surpass $4 trillion by 2035, www.worldobesity.org/news/economic-impact-of-overweight-and-obesity-to-surpass-4-trillion-by-2035 (2023).
2 Rubino, F. et al. Lancet Diabetes & Endocrinology Commission on the Definition and Diagnosis of Clinical Obesity. The Lancet Diabetes & Endocrinology 11, 226-228 (2023).
3 Acosta. A. et al. Selection of Antiobesity Medications Based on Phenotypes Enhances Weight Loss: A Pragmatic Trial in an Obesity Clinic. Obesity (Silver Spring) 29, 662-671 (2021).
4 Acosta, A. et al. Quantitative gastrointestinal and psychological traits associated with obesity and response to weight-loss therapy. Gastroenterology 148, 537-546.e534 (2015).
5 Halawi, H. et al. Effects of liraglutide on weight, satiation, and gastric functions in obesity: a randomised, placebo-controlled pilot trial. Lancet Gastroenterol Hepatol 2, 890-899 (2017).
6 Maselli, D. et al. Effects of liraglutide on gastrointestinal functions and weight in obesity: a randomized clinical and pharmacogenomic trial. Obesity 30, 1608-1620 (2022).
7 Blundell, J. & Halford, J. Regulation of nutrient supply: the brain and appetite control. Proceedings of the Nutrition Society 53, 407-418 (1994).
8 Koch, K. L. Gastric neuromuscular function and neuromuscular disorders. Sleisenger and Fordtran's gastrointestinal and liver disease: pathophysiology/diagnosis/management. Philadelphia: Elsevier, 789-815 (2010).
9 Camilleri, M. Peripheral mechanisms in appetite regulation. Gastroenterology 148, 1219-1233 (2015). 10Cifuentes, L. & Acosta. A. Homeostatic regulation of food intake. Clin Res Hepatol Gastroenterol 46, 101794 (2022).
11 Cummings, D. E. & Overduin, J. Gastrointestinal regulation of food intake. The Journal of clinical investigation 117, 13-23 (2007).
12 Blundell, J. et al. Appetite control: methodological aspects of the evaluation of foods. Obes Rev 11, 251-270 (2010).
13 Loos, R. J. Genetic determinants of common obesity and their value in prediction. Best Pract Res Clin Endocrinol Metab 26, 211-226 (2012).
14 Yengo, L. et al. Meta-analysis of genome-wide association studies for height and body mass index in ~700,000 individuals of European ancestry. Hum Mol Genet 27, 3641-3649 (2018).
15 Locke, A. E. et al. Genetic studies of body mass index yield new insights for obesity biology. Nature 518, 197-206 (2015).
16 Tabor. H. K., Risch, N. J. & Myers, R. M. Candidate-gene approaches for studying complex genetic traits: practical considerations. Nature Reviews Genetics 3, 391-397 (2002).
17 Elgart, M. et al. Non-linear machine learning models incorporating SNPs and PRS improve polygenic prediction in diverse human populations. Communications Biology 5, 856 (2022).
18 Levine, M. E., Langfelder, P. & Horvath, S. A weighted SNP correlation network method for estimating polygenic risk scores. Biological networks and pathway analysis, 277-290 (2017).
19 Yanovski, S. Z. & Yanovski, J. A. Progress in Pharmacotherapy for Obesity. JAMA 326, 129-130 (2021).
20 Atlas. S. J. et al. Medications for obesity management: Effectiveness and value. J Manag Care Spec Pharm 29, 569-575 (2023).
21 Nielsen, R. L. et al. Data integration for prediction of weight loss in randomized controlled dietary trials. Scientific reports 10 (2020).
22 Khera, R. et al. Association of pharmacological treatments for obesity with weight loss and adverse events: a systematic review and meta-analysis. Jama 315, 2424-2434 (2016).
23 Finer. N., Ryan, D., Renz. C. & Hewkin, A. Prediction of response to sibutramine therapy in obese non-diabetic and diabetic patients. Diabetes, Obesity and Metabolism 8, 206-213 (2006).
24 Guaraldi, F., Pagotto, U. & Pasquali, R. Predictors of weight loss and maintenance in patients treated with antiobesity drugs. Diabetes, metabolic syndrome and obesity: targets and therapy. 229-243 (2011).
25 Yearsley, J., Tolkamp, B. J. & Illius, A. W. Theoretical developments in the study and prediction of food intake. Proceedings of the Nutrition Society 60, 145-156 (2001).
26 Pedersen, H. et al. Predicting food intake from food reward and biometric responses to food cues in adults with normal weight using machine learning. The Journal of Nutrition 152, 1574-1581 (2022).
27 Rollins, B. Y., Loken, E. & Birch, L. L. Preferences predict food intake from 5 to 11 years, but not in girls with higher weight concerns, dietary restraint, and % body fat. Obesity 19, 2190-2197 (2011).
28 Vazquez-Roque, M. I. et al. Association of genetic variation in cannabinoid mechanisms and gastric motor functions and satiation in overweight and obesity. Neurogastroenterology & Motility 23, 637-e257 (2011).
29 Hasselbalch, A. L., Heitmann, B. L., Kyvik, K. O. & Sorensen, T. I. Studies of twins indicate that genetics influence dietary intake. The Journal of nutrition 138, 2406-2412 (2008).
30 Llewellyn, C. H., Trzaskowski, M., van Jaarsveld, C. H., Plomin, R. & Wardle, J. Satiety mechanisms in genetic risk of obesity. JAMA pediatrics 168, 338-344 (2014).
31 Chedid, V. et al. Allelic variant in the glucagon-like peptide 1 receptor gene associated with greater effect of liraglutide and exenatide on gastric emptying: A pilot pharmacogenetics study. Neurogastroenterol Motil 30, e13313 (2018).
32 Lagou, V. et al. GWAS of random glucose in 476.326 individuals provide insights into diabetes pathophysiology, complications and treatment stratification. Nat Genet 55, 1448-1461 (2023).
33 Elfhag, K. & Rossner, S. Who succeeds in maintaining weight loss?A conceptual review of factors associated with weight loss maintenance and weight regain. Obesity reviews 6, 67-85 (2005).
34 Williams, G. C., Grow, V. M., Freedman, Z. R., Ryan, R. M. & Deci, E. L. Motivational predictors of weight loss and weight-loss maintenance. Journal of personality and social psychology 70, 115 (1996).
35 Klein, M. L., Wing, R. R., McGuirc, M. T., Seagle, H. M. & Hill, J. O. A descriptive study of individuals successful at long-term maintenance of substantial weight loss. The American journal of clinical nutrition 66, 239-246 (1997).
36 Saux, P. et al. Development and validation of an interpretable machine learning-based calculator for predicting 5-year weight trajectories after bariatric surgery: a multinational retrospective cohort SOPHIA study. The Lancet Digital Health 5, e692-e702 (2023).
37 Homer, K. M., Byrne, N. M. & King, N. A. Reproducibility of subjective appetite ratings and ad libitum test meal energy intake in overweight and obese males. Appetite 81, 116-122 (2014).
38 Camilleri, M. et al. Performance characteristics of scintigraphic measurement of gastric emptying of solids in healthy participants. Neurogastroenterology & Motility 24, 1076-e1562 (2012).
39 Bouras, E. et al. SPECT imaging of the stomach: comparison with barostat, and effects of sex, age, body mass index, and fundoplication. Gut 51, 781 (2002).
40 Angle, S. et al. Three factor eating questionnaire-R18 as a measure of cognitive restraint, uncontrolled eating and emotional eating in a sample of young Finnish females. International Journal of Behavioral Nutrition and Physical Activity 6, 1-7 (2009).
41 Yeo. I. K. & Johnson, R. A. A New Family of Power Transformations to Improve Normality or Symmetry. Biometrika 87, 954-959 (2,000).
42 Chawla, N. V., Bowyer, K. W., Hall, L. O. & Kegelmeyer, W. P. SMOTE: synthetic minority over-sampling technique. J. Artif. Int. Res. 16, 321-357 (2002).
43 Abu Dayyeh, Barham K, George Woodman, Andres Acosta, Guilherme S Lopes, Christopher Gostout, Eduardo G de Moura, Victoria Gomez, Bruno da Costa Martins, Mark Topazian, and Michael Camilleri. 2016. “Baseline gastric emptying and its change in response to diverse endoscopic bariatric therapies predict weight change after intervention.” In Gastroenterology, S86-S86. W B SAUNDERS C O-ELSEVIER INC 1600 JOHN F KENNEDY BOULEVARD, STE 1800.
44 Ames, Gretchen E, Michael G Heckman, Nancy N Diehl, Karen B Grothe, and Matthew M Clark. 2015. ‘Further statistical and clinical validity for the weight efficacy lifestyle questionnaire-short form’, Eating behaviors. 18: 115-19.
45 Campos A, Cifuentes L, Hashem A, Busebee B. Hurtado-Andrade M D, Ricardo-Silgado M L, McRae A. De la Rosa A, Feris F, Bublitz J T, Hensrud D, Camilleri M, Kellogg T A, Eckel-Passow J E, Olson J, Acosta A. Effects of Heterozygous Variants in the Leptin-Melanocortin Pathway on Roux-en-Y Gastric Bypass Outcomes: a 15-Year Case-Control Study. Obes Surg. 2022 August; 32(8):2632-2640.
46 Cifuentes, Lizeth, Wissam Ghusn, Fauzi Feris, Alejandro Campos. Daniel Sacoto, Alan De la Rosa, Alison McRae, Thom Rieck, Sara Mansfield, and Jason Ewoldt. 2023. ‘Phenotype tailored lifestyle intervention on weight loss and cardiometabolic risk factors in adults with obesity: a single-centre, non-randomised, proof-of-concept study’, EClinicalMedicine, 58.
47 Clark, Matthew M. David B Abrams, Raymond S Niaura, Cheryl A Eaton, and Joseph S Rossi. 1991. ‘Self-efficacy in weight management’, Journal of consulting and clinical psychology, 59: 739.
48 Halawi, H., M. Camilleri, A. Acosta. M. Vazquez-Roque, I. Oduyebo, D. Burton, I. Busciglio, and A. R. Zinsmeister. 2017. ‘Relationship of gastric emptying or accommodation with satiation, satiety, and postprandial symptoms in health’, Am J Physiol Gastrointest Liver Physiol, 313: G442-g47.
49 Hill, James O, Holly R Wyatt, and John C Peters. 2012. ‘Energy balance and obesity’, Circulation. 126: 126-32.
50. Hurtado M D. Cifuentes L, Campos A, De La Rosa A. Kapoor E, Faubion S S, Hensrud D D, Camilleri M. Acosta A. Sex as an independent variable in the measurement of satiation: a retrospective cohort study. Int J Obes (Lond). 2022 December; 46(12):2156-2162
51 Karlsson, J., L. O. Persson, L. Sjöström, and M. Sullivan. 2,000. ‘Psychometric properties and factor structure of the Three-Factor Eating Questionnaire (TFEQ) in obese men and women. Results from the Swedish Obese Subjects (SOS) study’. Int. J Obes Relat Metab Disord, 24: 1715-25.
52 Kim, Gwang-Hyun, Jeng-Haw Lin, E S Blomain, and S. Waldman. 2013. ‘Antiobesity Pharmacotherapy: New Drugs and Emerging Targets’, Clinical Pharmacology & Therapeutics, 95.

### Example 5—Performance of a Machine-Learning Gene Risk Score Biomarker on Predicting Response to Semaglutide: a Prospectively Followed Multi-Center Biobank and Outcomes Registry

### Background and Objective

The phenotype-guided approach to anti-obesity medications as presented throughout this specification is associated with greater weight loss compared to the obesity medicine standard of care. For instance, the abnormal post-prandial satiety phenotype (“hungry gut”-HG) is associated with greater weight loss in response to GLP-1 receptor agonists (GLP-1RA) liraglutide and exenatide that are known to prolong post-prandial satiety. As described previously herein (see Examples 2 and 4), a machine-learning assisted gene risk score (ML-GRS) was developed to predict HG such that those determined to possess a low CTS_{GRS} were considered HG and validated it in patients taking liraglutide against placebo in a randomized controlled trial. The ML-GRS was developed by exploring the associations between variables related to food intake regulation and single nucleotide polymorphisms within an expert-guided set of 40 genes. The model predicted the response to liraglutide with an AUC of 0.69 (DOI:10.1016/S0016-5085(23)01311-2).

The goal of this Example was to determine the effect of this ML-GRS in predicting weight loss response to semaglutide, a more effective GLP-1 RA, which is still unknown.

### Methods

A multi-center biobank and outcomes registry of adults undergoing weight loss interventions at Mayo Clinic (IRB: 21-011737) was established. The registry collects information on demographics, anthropometrics, and weight loss interventions and their outcomes. For this report, individuals with obesity prescribed semaglutide 0.25-2.4 mg were enrolled. Using saliva or blood samples, genetic studies were performed and a ML-GRS was generated, a continuous variable varying from 0 to 1. The analysis was divided as following: 1) ML-GRS<0.50 (“Hungry Gut Positive” [HG+]), and 2) ML-GRS≥0.50 (“Hungry Gut Negative” [HG-]). The endpoints were the total body weight loss (TBWL) % at 3, 6, 9 and 12 months and the probability of the ML-GRS to predict response to semaglutide, defined as TBWL≥5% at 12 months. Continuous data were analyzed using paired t-test and categorical data using Fisher's exact test, with p-value <0.05 considered statistically significant. Results are presented as mean±standard deviation.

### Results

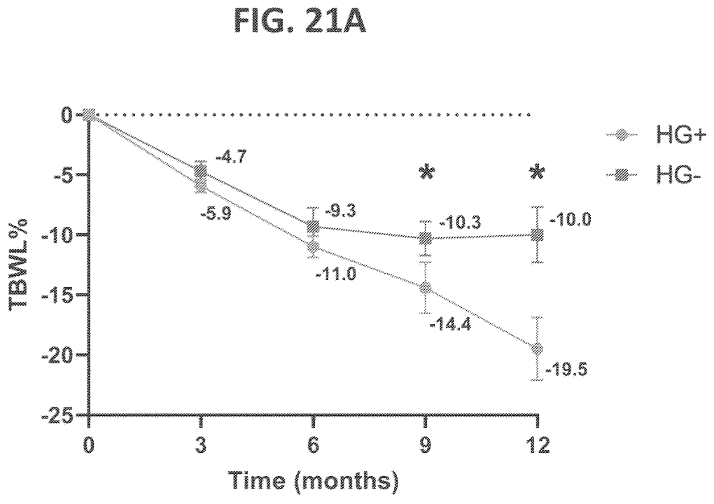

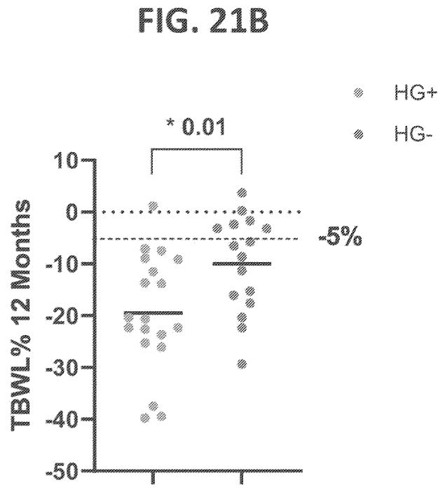

84 participants were included: age 47.6±10.9. BMI 38.8±6.9 kg/m2 (Table 18). Compared to HG−, HG+ had superior TBWL % at 9 months (−14.4±6.6% vs. −10.3±7.0%, p=0.045) and at 12 months (−19.5±11.4% vs. −10.0±9.3%, p=0.01) but not at 3 and 6 months (FIGS. 21A and 21B). When used to predict response, the ML-GRS had an AUC of 0.76 (95% CI [0.57-0.94], p=0.04), with a PPV equal to 0.95.

### CONCLUSIONS

The HG ML-GRS developed and described herein could serve as a biomarker that predicts response to semaglutide. As such, the HG ML-PRS test could be employed in clinical practice to select responders to semaglutide, thereby reducing obesity heterogeneity.

TABLE 18
Demographics, baseline body composition, baseline T2D status,
and medication dose of participants taking semaglutide.
Abbreviations: ML-GRS: Machine Learning-Gene Risk Score

| | Hungry Gut | Hungry Gut | |
|---|---|---|---|
| | Positive | Negative | |
| | (ML-GRS < 0.50) | (ML-GRS ≥ 0.50) | p- |
| | N = 51 | N = 33 | value |
| Demographics | | | |
| Age, years (SD) | 48.7 (12.0) | 46.9 (10.2) | 0.46 |
| Sex. females (%) | 80% | 79% | 1.00 |
| Race, Caucasian (%) | 94% | 94% | 1.00 |
| Baseline Body | | | |
| Composition (SD) | | | |
| Weight, kg | 110.1 (23.4) | 110.8 (24.5) | 0.89 |
| Height, m | 1.68 (0.09) | 1.69 (0.08) | 0.38 |
| Body mass index, | 39.0 (6.3) | 38.6 (7.7) | 0.83 |
| kg/m² | | | |
| Baseline | | | |
| comorbidities, N (%) | | | |
| Type 2 Diabetes | 8 (16%) | 11 (33%) | 0.07 |
| Maximum dose | | | |
| achieved, N (%) | | | |
| 0.25-1.00 mg | 7 (14%) | 8 (24%) | 0.26 |
| 1.70-2.40 mg | 44 (86%) | 25 (76%) | |

### Numbered Embodiments of the Disclosure

Other subject matter contemplated by the present disclosure is set out in the following numbered embodiments:
1. A computer-implemented method for generating a machine learning assisted gene risk score (ML-GRS) for predicting an obesity phenotype of interest in a subject suffering from obesity, the method comprising:
   (a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein each gene in the set of genes is known or suspected to play a role in obesity;
   (b) generating by the computer system, a contribution factor for each SNP located in, around or near a gene from the set of genes from the genetic dataset for a subject from the population of subjects, wherein the contribution factor is a numerical value, vector, matrix, or function of various biological factors that represents the contribution or effect that each SNP has on a specific trait known to be associated with the obesity phenotype of interest;
   (c) calculating by the computer system, a gene risk score (GRS) for each gene in the set of genes by integrating the contribution factors generated in step (b) for each SNP for each gene from the set of genes and SNP genotype information located in, around, or near each gene in the set of genes, wherein the GRS for each gene represents the cumulative effect of the variation in SNPs located in, around, or near the gene in that particular subject has on the function or regulation of the gene so as to increase or decrease the risk of the subject possessing a specific trait known to be associated with the obesity phenotype of interest;
   (d) iteratively performing by the computer system, steps (a)-(c) for each additional subject from the population of subjects to generate a set of GRSs for each gene in the set of genes from the population of subjects, wherein each gene has a GRS for each sex;
   (e) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts if any one subject possesses the specific trait known to be associated with the obesity phenotype of interest by combining gene risk scores and additional biological, psychological, or environmental measurements of the any one subject;
   (f) training by the computer system, the ML-GRS from step (e) using a classifier model to predict subjects from a training set of samples from a training population of obese subjects as being positive for the specific trait known to be associated with the obesity phenotype of interest, thereby generating a trained ML-GRS; and
   (g) determining by the computer system, if a test subject is positive for the specific trait known to be associated with the obesity phenotype of interest by:
      (i) calculating by the computer system the ML-GRS for the test subject using steps (a)-(e) on a sample obtained from the test subject; and
      (ii) comparing by the computer system, the ML-GRS for the test subject to the trained ML-GRS, wherein the test subject is positive for the specific trait known to be associated with the obesity phenotype of interest if the ML-GRS for the subject is above the sex-specific 75^{th} percentile for the specific trait known to be associated with the obesity phenotype of interest or negative for the specific trait known to be associated with the obesity phenotype of interest if the ML-GRS for the subject is below the sex-specific 75^{th} percentile for the specific trait known to be associated with the obesity phenotype of interest.
2. The computer-implemented method of embodiment 1, wherein the SNP genotype information comprises risk allele dosage, major allele presence, occurrence of a de novo variant, insertion, deletion, or genome rearrangement.
3. The computer-implemented method of embodiment 1 or 2, wherein each gene in the set of genes is known or suspected to play a role in appetite regulation, energy expenditure, lipid metabolism or adipogenesis.
4. The computer-implemented method of embodiment 1 or 2, wherein the subject is obese if the subject possesses a body mass index (BMI)>30 kg/m² with or without type 2 diabetes.
5. The computer-implemented method of any one of the above embodiments, wherein the ML-GRS for the obesity phenotype of interest predicts the specific trait known to be associated with the obesity phenotype of interest with a sensitivity and/or specificity is at least 65%.
6. The computer-implemented method of any one of the above embodiments, wherein the ML-GRS predicts the specific trait known to be associated with the obesity phenotype of interest with an area under the curve (AUC) of at least 0.65, 0.7, 0.75, 0.8, 0.85, 0.9 0.95 or 1.
7. The computer-implemented method of any one of embodiments 1-5, wherein the ML-GRS predicts the specific trait known to be associated with the obesity phenotype of interest with positive predictive value of at least 50%.
8. The computer-implemented method of any one of the above embodiments, wherein the contribution factor is a function or algorithm that utilizes output of a regression analysis or a statistical analysis on the genetic dataset as well as one or more additional parameters, covering regression against one or more traits using one or more types of genetic datasets selected from the group consisting of a genome-wide data microarray chip (GWAS), targeted sequencing (exome or targeted genetic panel), variants detected by qPCR through targeted amplification, whole genome sequencing (WGS), targeted or untargeted sequencing of genomic re-arrangement, deletions, duplications, repeat extensions, and complex genotyping of HLA and CYP genes.
9. The computer-implemented method of embodiment 8, wherein the one or more additional parameters relate to biological factors regarding genetics, epigenetics, and gene regulation for each SNP.
10. The computer-implemented method of embodiment 9, wherein the one or more additional parameters are selected from the group consisting of proximity of the SNP to the specific gene, known or theoretical mechanistic role of the SNP metadata regarding the SNP in relationship to the gene, and any combination thereof.
11. The computer-implemented method of embodiment 10, wherein the known or theoretical mechanistic role of the SNP is selected from the group consisting of synonymous mutation, nonsynonymous mutation, frameshift mutation and nonsense mutation.
12. The computer-implemented method of embodiment 11, wherein the known or theoretical mechanistic role of the SNP affects the three-dimensional structure of the translated protein binding sites, protein-protein interaction sites and modification sites, wherein the modification sites are selected from the group consisting of phosphorylation, glycosylation and proteolytic site.
13. The computer-implemented method of embodiment 10, wherein the mechanistic role comprises whether or not the SNP is within an intron, exon, enhancer region or regulatory region of the gene, a cis-regulatory element, a promoter region, a non-coding exonic region, a coding exonic region, intronic region, splice site, transcription factor binding site, epigenetic modification site, at a remote genomic location involved in a three-dimensional chromatin contact with the gene, or a cell-type-specific topological-associated domain that contains the gene.
14. The computer-implemented method of embodiment 13, wherein the cis-regulatory element is an enhancer or insulator.
15. The computer-implemented method of embodiment 13, wherein the epigenetic modification site comprises DNA methylation modifications, histone modifications, and/or chromatin accessibility.
16. The computer-implemented method of any one of the above embodiments, wherein the non-genetic and/or multi-omic data is selected from the group consisting of metabolomic data, proteomic data, peptidomic data, epigenetic data, microbiome data, results from one or more questionnaires and any combination thereof.
17. The computer-implemented method of embodiment 16, wherein the epigenetic data comprises a presence or absence of DNA modifications selected from the group consisting of DNA methylation modifications, histone modifications and chromatin accessibility.
18. The computer-implemented method of embodiment 16, wherein the microbiome data is from any site on the subject or the subject's environment.
19. The computer-implemented method of any one of the above embodiments, wherein the machine learning model in step (e) comprises a forward feature selection, backward feature selection, or random feature sampling algorithm and a random forest, GBM, or linear predictor that iteratively selects a feature with a desired training accuracy using the random forest predictor in multiple rounds until no further training accuracy improvement is detected.
20. The computer-implemented method of any one of the above embodiments, wherein the GRSs of step (e) are normalized.
21. The computer-implemented method of any one of the above embodiments, wherein each GSR provides an indication of a role or effect that the SNPs located around or near a specific gene may play on the specific trait known to be associated with the obesity phenotype of interest.
22. The computer-implemented method of any one of the above embodiments, wherein the SNP is considered to be located around or near a specific gene if it is located within at least 500,000 kilobases (kb) of the specific gene.
23. The computer-implemented method of any one of the above embodiments, further comprising performing a genetic analysis on a sample obtained from the subject suffering from or suspected of suffering from obesity prior to step (a).
24. The computer-implemented method of embodiment 23, wherein the genetic analysis comprises obtaining sequence reads from the whole or portions of the whole genome of the subject.
25. The computer-implemented method of embodiment 23, wherein the genetic analysis comprises performing a genotyping method selected from the group consisting of restriction fragment length polymorphism identification (RFLPI), random amplified polymorphic detection (RAPD), amplified fragment length polymorphism detection (AFLPD), polymerase chain reaction (PCR), DNA sequencing. RNA sequencing, allele specific oligonucleotide (ASO) probes, and hybridization to microarrays or beads.
26. The computer-implemented method of any one of the above embodiments, further comprising performing by the computer system, a regression analysis and/or statistical analysis on the genetic dataset from step (a) prior to step (b).
27. The computer-implemented method of embodiment 26, wherein the regression analysis is a ridge regression or least absolute shrinkage and selection operator (LASSO) regression.
28. The computer-implemented method of embodiment 26, wherein beta values obtained from the regression analyses performed on the genetic datasets are used to generate the contribution factor in step (b).
29. The computer-implemented method of embodiment 26, wherein the statistical analysis is a genome-wide association study (GWAS).
30. The computer-implemented method of embodiment 29, wherein p-values and/or regression weights obtained from the statistical analyses performed on the genetic datasets are used to generate the contribution factors in step (b).
31. The computer-implemented method of any one of the above embodiments, wherein the obesity phenotype of interest is selected from the group consisting of hungry brain (abnormal satiation), hungry gut (abnormal satiety), slow burn (slow metabolism) or hedonic/emotional eating).
32. The computer-implemented method of any one of the above embodiments, wherein the obesity phenotype of interest is a hungry brain (abnormal satiation) or hungry gut (abnormal satiety).
33. The computer-implemented method of embodiment 32, wherein the specific trait in step (b) is total kcal consumed to satiation (CTS) at an ad libitum meal.
34. The computer-implemented method of embodiment 32, wherein following step (e), the subject is CTS positive if the subject has a ML-GRS for CTS above the sex-specific seventy-fifth percentile or the 4′ quartile, while the subject is CTS negative if the subject has a ML-GRS for CTS below the sex-specific seventy-fifth percentile.
35. The computer-implemented method of embodiment 34, wherein the plurality of genes in step (a) are two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1. FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, TNFRSF11A or DYRK1B.
36. The method of any one of embodiments 1-31, wherein the obesity phenotype of interest is a hungry gut (abnormal satiety).
37. The method of embodiment 36, wherein the specific trait is increased or accelerated baseline gastric emptying for the subject as compared to a control subject, wherein the control subject is not obese.
38. The method of embodiment 37, wherein the set of genes in step (a) consists of genes known to play role in conferring the hungry gut phenotype.
39. The method of any one of embodiments 1-31, wherein the obesity phenotype of interest is a slow burn.
40. The method of embodiment 39, wherein the specific trait is decreased or low resting energy expenditure (REE) for the subject as compared to a control, wherein the control subject is not obese.
41. The method of embodiment 40, wherein the set of genes in step (a) consists of genes known to play role in conferring the slow burn phenotype.
42. The method of any one of embodiments 1-31, wherein the obesity phenotype of interest is an emotional eating phenotype.
43. The method of embodiment 42, wherein the specific trait is a finding or result from a behavioral questionnaire indicative of anxiety or emotional eating for the subject.
44. The method of embodiment 43, wherein the questionnaire is the Hospital Anxiety and Depression Scale (HADS) questionnaire.
45. The method of embodiment 42, wherein the plurality of genes in step (a) consists of genes known to play role in conferring the emotional eating phenotype.
46. A computer-implemented method for predicting or diagnosing a subject suffering from or suspected of suffering from obesity as possessing abnormal satiation, the method comprising:
   (a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein the set of genes comprises GLP1R, UCP2, FTO, TCF7L2, MBOAT4 (GOAT), ADRA2A, GNB3, GPBAR1, LEP, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, TNFRSF11A and DYRK1B;
   (b) generating by the computer system, a contribution factor for each SNP located in or near a gene from the set of genes from the genetic dataset for a subject from the population of subjects, wherein the contribution factor is a numerical value, vector, matrix, or function of various biological factors that represents the contribution or effect that each SNP has on total kcal consumed to satiation (CTS) at an ad libitum meal;
   (c) calculating by the computer system, a gene risk score (GRS) for each gene in the set of genes by integrating the contribution factors generated in step (b) for each SNP for each gene from the set of genes and SNP genotype information located in, around or near each gene in the set of genes, wherein the GRS for each gene represents the cumulative effect of the variation in SNPs located in, around, or near the gene in that particular subject has on the function or regulation of the gene so as to increase or decrease the risk of being above a sex-specific threshold total kcal consumed to satiation (CTS) at an ad libitum meal;
   (d) iteratively performing by the computer system, steps (a)-(c) for each additional subject from the population of subjects to generate a set of GRSs for each gene in the set of genes, wherein each gene has a GRS for each sex; and
   (e) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts the CTS by combining gene risk scores and other biological, psychological, or environmental measurements of the subject;
   (f) training by the computer system, the ML-GRS from step (e) using a classifier model to predict subjects from a training set of samples from a training population of obese subjects as being positive for being above a sex-specific threshold total kcal consumed to satiation (CTS) at an ad libitum meal, thereby generating a trained ML-GRS; and
   (g) determining by the computer system, if a test subject is positive for the specific trait known to be associated with the obesity phenotype of interest by:
      (i) calculating by the computer system the ML-GRS for the test subject using steps (a)-(e) on a sample obtained from the test subject; and
      (ii) comparing by the computer system, the ML-GRS for the test subject to the trained ML-GRS, wherein the test subject is positive for CTS if the ML-GRS for the subject is above the sex-specific 75^{th} percentile for CTS or negative for CTS if the ML-GRS for the subject is below the sex-specific 75^{th} percentile for CTS.
47. The method of embodiment 46, wherein the sample is selected from the group consisting of a blood sample, a saliva sample, a urine sample, a breath sample, and a stool sample.
48. A method for treating obesity in a subject in need thereof, the method comprising:
   (a) determining if the subject possesses abnormal satiation using the method of embodiment 46 on a genetic dataset obtained from a sample obtained the subject to determine a ML-GRS for abnormal satiation; and
   (b) administering a pharmacotherapy to the subject that does not include a GLP-1 agonist if the ML-GRS determined for the subject indicates that the subject is positive for CTS or administering a pharmacotherapy that may include a GLP-1 agonist to the subject if the ML-GRS determined for the subject indicates that the subject is negative for CTS.
49. A method for treating obesity in a subject in need thereof, the method comprising:
   (a) determining the presence, absence or level of a plurality of gastrointestinal (GI) peptides, a plurality of metabolites, and/or a plurality of genetic variants in a sample obtained from a subject suffering from obesity as well as satiety, satiation, resting energy expenditure and results on a behavioral questionnaire for the subject, thereby generating an obesity analyte signature for the sample;
   (b) populating a predictive machine learning model with the obesity analyte signature for the subject; and
   (c) utilizing the predictive machine learning model to predict an obesity phenotype of the subject suffering from obesity, wherein the obesity phenotype is selected from the group consisting of hungry brain (abnormal satiation), hungry gut (abnormal satiety), slow burn (slow metabolism) or hedonic/emotional eating); and
   (d) administering a pharmacotherapy to the subject that does not include a GLP-1 agonist if the predictive machine learning model to predicts that the subject possesses an abnormal satiation phenotype or administering a pharmacotherapy that may include a GLP-1 agonist to the subject if the predictive machine learning model to predicts that the subject does not possess an abnormal satiation phenotype.
50. The method of embodiment 48 or 49, wherein the pharmacotherapy that does not include a GLP-1 agonist is phentermine/topiramate.
51. The method of embodiment 48 or 49, wherein the pharmacotherapy that does include a GLP-1 agonist is selected from the group consisting of exenatide, liraglutide, albiglutide, dulaglutide, lixisenatide, semaglutide and tirzepatide.
52. A method of treating obesity in a subject in need thereof, the method comprising:
   (a) detecting an obesity analyte signature in a sample obtained from a subject; wherein the obesity analyte signature is indicative of an obesity phenotype of the subject; and
   (b) administering a pharmacotherapy to the subject that does not include a GLP-1 agonist if the obesity analyte signature detected in the sample obtained from the subject indicates that the subject possesses an abnormal satiation phenotype or administering a pharmacotherapy that may include a GLP-1 agonist to the subject if the obesity analyte signature detected in the sample obtained from the subject indicates that the subject does not possess an abnormal satiation phenotype.
53. The method of embodiment 52, wherein the GLP-1 agonist is selected from the group consisting of exenatide, liraglutide, albiglutide, dulaglutide, lixisenatide, semaglutide and tirzepatide.
54. A method of identifying a subject suffering from obesity as a non-responder to treatment with a GLP-1 agonist, the method comprising detecting an obesity phenotype of the subject; and identifying the subject as a non-responder to GLP-1 agonist treatment if the subject is determined to possess an abnormal satiation obesity phenotype.
55. The method of any of one of embodiments 49-53, wherein the sample is selected from the group consisting of a blood sample, a saliva sample, a urine sample, a breath sample, and a stool sample.
56. The method of any one of embodiments 52-55, wherein the obesity analyte signature comprises the presence of serotonin, glutamine, isocaproic acid, allo-isoleucine, hydroxyproline, beta-aminoisobutyric-acid, alanine, hexanoic acid, tyrosine, and PYY, an absence of 1-methylhistine, gamma-amino-n-butyric-acid, phenylalanine, ghrelin, and a HADS questionnaire result that does not indicate an anxiety subscale (HADS-A: e.g., includes a HADS-A questionnaire result).
57. The method of embodiment 56, wherein the subject consumes above the seventy-fifth percentile amount of calories at an ad libitum meal.
58. The method of any one of embodiments 52-55, wherein the obesity analyte signature comprises the presence of 1-methylhistine, allo-isoleucine, hydroxyproline, beta-aminoisobutyric-acid, alanine, and phenylalanine, an absence of serotonin, glutamine, gamma-amino-n-butyric-acid, isocaproic acid, hexanoic acid, tyrosine, ghrelin, PYY, and does not include a HADS questionnaire result that indicates an anxiety subscale.
59. The method of embodiment 58, wherein the subject has increased or accelerated baseline gastric emptying as compared to a control subject, wherein the control subject is not obese.
60. The method of any one of embodiments 46-49, wherein the obesity analyte signature comprises the presence of 1-methylhistine, serotonin, glutamine, gamma-amino-n-butyric-acid, isocaproic acid, allo-isoleucine, alanine, tyrosine, ghrelin, PYY, an absence of hydroxyproline, beta-aminoisobutyric-acid, hexanoic acid, and phenylalanine, and does includes a HADS questionnaire result that indicates an anxiety subscale.
61. The method of embodiment 52, wherein the subject has decreased or low resting energy expenditure (REE) for the subject as compared to a control, wherein the control subject is not obese.
62. The method of any one of embodiments 52-55, wherein the obesity analyte signature comprises the presence of serotonin, an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and does includes a HADS questionnaire result that indicates an anxiety subscale.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed:

1. A computer-implemented method for generating a machine learning assisted gene risk score (ML-GRS) for predicting an obesity phenotype of interest in a subject suffering from obesity, the method comprising:
(a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein each gene in the set of genes is known or suspected to play a role in obesity;
(b) generating by the computer system, a contribution factor for each SNP located in, around or near a gene from the set of genes from the genetic dataset for a subject from the population of subjects, wherein the contribution factor is a numerical value, vector, matrix, or function of various biological factors that represents the contribution or effect that each SNP has on a specific trait known to be associated with the obesity phenotype of interest;
(c) calculating by the computer system, a gene risk score (GRS) for each gene in the set of genes by integrating the contribution factors generated in step (b) for each SNP for each gene from the set of genes and SNP genotype information located in, around, or near each gene in the set of genes, wherein the GRS for each gene represents the cumulative effect of the variation in SNPs located in, around, or near the gene in that particular subject has on the function or regulation of the gene so as to increase or decrease the risk of the subject possessing a specific trait known to be associated with the obesity phenotype of interest;
(d) iteratively performing by the computer system, steps (a)-(c) for each additional subject from the population of subjects to generate a set of GRSs for each gene in the set of genes from the population of subjects, wherein each gene has a GRS for each sex;
(e) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts if any one subject possesses the specific trait known to be associated with the obesity phenotype of interest by combining gene risk scores and additional biological, psychological, or environmental measurements of the any one subject;
(f) training by the computer system, the ML-GRS from step (e) using a classifier model to predict subjects from a training set of samples from a training population of obese subjects as being positive for the specific trait known to be associated with the obesity phenotype of interest, thereby generating a trained ML-GRS; and
(g) determining by the computer system, if a test subject is positive for the specific trait known to be associated with the obesity phenotype of interest by:
(i) calculating by the computer system the ML-GRS for the test subject using steps (a)-(e) on a sample obtained from the test subject; and
(ii) comparing by the computer system, the ML-GRS for the test subject to the trained ML-GRS, wherein the test subject is positive for the specific trait known to be associated with the obesity phenotype of interest if the ML-GRS for the subject is above the sex-specific 75^{th} percentile for the specific trait known to be associated with the obesity phenotype of interest or negative for the specific trait known to be associated with the obesity phenotype of interest if the ML-GRS for the subject is below the sex-specific 75^{th} percentile for the specific trait known to be associated with the obesity phenotype of interest.

2. The computer-implemented method of claim 1, wherein the SNP genotype information comprises risk allele dosage, major allele presence, occurrence of a de novo variant, insertion, deletion, or genome rearrangement.

3. The computer-implemented method of claim 1 or 2, wherein each gene in the set of genes is known or suspected to play a role in appetite regulation, energy expenditure, lipid metabolism or adipogenesis.

4. The computer-implemented method of claim 1 or 2, wherein the subject is obese if the subject possesses a body mass index (BMI)>30 kg/m² with or without type 2 diabetes.

5. The computer-implemented method of any one of the above claims, wherein the ML-GRS for the obesity phenotype of interest predicts the specific trait known to be associated with the obesity phenotype of interest with a sensitivity and/or specificity is at least 65%.

6. The computer-implemented method of any one of the above claims, wherein the ML-GRS predicts the specific trait known to be associated with the obesity phenotype of interest with an area under the curve (AUC) of at least 0.65, 0.7, 0.75, 0.8, 0.85, 0.9 0.95 or 1.

7. The computer-implemented method of any one of claims 1-5, wherein the ML-GRS predicts the specific trait known to be associated with the obesity phenotype of interest with positive predictive value of at least 50%.

8. The computer-implemented method of any one of the above claims, wherein the contribution factor is a function or algorithm that utilizes output of a regression analysis or a statistical analysis on the genetic dataset as well as one or more additional parameters, covering regression against one or more traits using one or more types of genetic datasets selected from the group consisting of a genome-wide data microarray chip (GWAS), targeted sequencing (exome or targeted genetic panel), variants detected by qPCR through targeted amplification, whole genome sequencing (WGS), targeted or untargeted sequencing of genomic re-arrangement, deletions, duplications, repeat extensions, and complex genotyping of HLA and CYP genes.

9. The computer-implemented method of claim 8, wherein the one or more additional parameters relate to biological factors regarding genetics, epigenetics, and gene regulation for each SNP.

10. The computer-implemented method of claim 9, wherein the one or more additional parameters are selected from the group consisting of proximity of the SNP to the specific gene, known or theoretical mechanistic role of the SNP metadata regarding the SNP in relationship to the gene, and any combination thereof.

11. The computer-implemented method of claim 10, wherein the known or theoretical mechanistic role of the SNP is selected from the group consisting of synonymous mutation, nonsynonymous mutation, frameshift mutation and nonsense mutation.

12. The computer-implemented method of claim 11, wherein the known or theoretical mechanistic role of the SNP affects the three-dimensional structure of the translated protein binding sites, protein-protein interaction sites and modification sites, wherein the modification sites are selected from the group consisting of phosphorylation, glycosylation and proteolytic site.

13. The computer-implemented method of claim 10, wherein the mechanistic role comprises whether or not the SNP is within an intron, exon, enhancer region or regulatory region of the gene, a cis-regulatory element, a promoter region, a non-coding exonic region, a coding exonic region, intronic region, splice site, transcription factor binding site, epigenetic modification site, at a remote genomic location involved in a three-dimensional chromatin contact with the gene, or a cell-type-specific topological-associated domain that contains the gene.

14. The computer-implemented method of claim 13, wherein the cis-regulatory element is an enhancer or insulator.

15. The computer-implemented method of claim 13, wherein the epigenetic modification site comprises DNA methylation modifications, histone modifications, and/or chromatin accessibility.

16. The computer-implemented method of any one of the above claims, wherein the non-genetic and/or multi-omic data is selected from the group consisting of metabolomic data, proteomic data, peptidomic data, epigenetic data, microbiome data, results from one or more questionnaires and any combination thereof.

17. The computer-implemented method of claim 16, wherein the epigenetic data comprises a presence or absence of DNA modifications selected from the group consisting of DNA methylation modifications, histone modifications and chromatin accessibility.

18. The computer-implemented method of claim 16, wherein the microbiome data is from any site on the subject or the subject's environment.

19. The computer-implemented method of any one of the above claims, wherein the machine learning model in step (e) comprises a forward feature selection, backward feature selection, or random feature sampling algorithm and a random forest, GBM, or linear predictor that iteratively selects a feature with a desired training accuracy using the random forest predictor in multiple rounds until no further training accuracy improvement is detected.

20. The computer-implemented method of any one of the above claims, wherein the GRSs of step (e) are normalized.

21. The computer-implemented method of any one of the above claims, wherein each GSR provides an indication of a role or effect that the SNPs located around or near a specific gene may play on the specific trait known to be associated with the obesity phenotype of interest.

22. The computer-implemented method of any one of the above claims, wherein the SNP is considered to be located around or near a specific gene if it is located within at least 500,000 kilobases (kb) of the specific gene.

23. The computer-implemented method of any one of the above claims, further comprising performing a genetic analysis on a sample obtained from the subject suffering from or suspected of suffering from obesity prior to step (a).

24. The computer-implemented method of claim 23, wherein the genetic analysis comprises obtaining sequence reads from the whole or portions of the whole genome of the subject.

25. The computer-implemented method of claim 23, wherein the genetic analysis comprises performing a genotyping method selected from the group consisting of restriction fragment length polymorphism identification (RFLPI), random amplified polymorphic detection (RAPD), amplified fragment length polymorphism detection (AFLPD), polymerase chain reaction (PCR), DNA sequencing, RNA sequencing, allele specific oligonucleotide (ASO) probes, and hybridization to microarrays or beads.

26. The computer-implemented method of any one of the above claims, further comprising performing by the computer system, a regression analysis and/or statistical analysis on the genetic dataset from step (a) prior to step (b).

27. The computer-implemented method of claim 26, wherein the regression analysis is a ridge regression or least absolute shrinkage and selection operator (LASSO) regression.

28. The computer-implemented method of claim 26, wherein beta values obtained from the regression analyses performed on the genetic datasets are used to generate the contribution factor in step (b).

29. The computer-implemented method of claim 26, wherein the statistical analysis is a genome-wide association study (GWAS).

30. The computer-implemented method of claim 29, wherein p-values and/or regression weights obtained from the statistical analyses performed on the genetic datasets are used to generate the contribution factors in step (b).

31. The computer-implemented method of any one of the above claims, wherein the obesity phenotype of interest is selected from the group consisting of hungry brain (abnormal satiation), hungry gut (abnormal satiety), slow burn (slow metabolism) or hedonic/emotional eating).

32. The computer-implemented method of any one of the above claims, wherein the obesity phenotype of interest is a hungry brain (abnormal satiation) or hungry gut (abnormal satiety).

33. The computer-implemented method of claim 32, wherein the specific trait in step (b) is total kcal consumed to satiation (CTS) at an ad libitum meal.

34. The computer-implemented method of claim 32, wherein following step (e), the subject is CTS positive if the subject has a ML-GRS for CTS above the sex-specific seventy-fifth percentile or the 4^{th} quartile, while the subject is CTS negative if the subject has a ML-GRS for CTS below the sex-specific seventy-fifth percentile.

35. The computer-implemented method of claim 34, wherein the plurality of genes in step (a) are two or more genes selected from GLP1R, UCP2, FTO, TCF7L2, MBOAT4 (GOAT), ADRA2A, GNB3, GPBAR1, LEP, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, TNFRSF11A or DYRK1B.

36. The method of any one of claims 1-31, wherein the obesity phenotype of interest is a hungry gut (abnormal satiety).

37. The method of claim 36, wherein the specific trait is increased or accelerated baseline gastric emptying for the subject as compared to a control subject, wherein the control subject is not obese.

38. The method of claim 37, wherein the set of genes in step (a) consists of genes known to play role in conferring the hungry gut phenotype.

39. The method of any one of claims 1-31, wherein the obesity phenotype of interest is a slow burn.

40. The method of claim 39, wherein the specific trait is decreased or low resting energy expenditure (REE) for the subject as compared to a control, wherein the control subject is not obese.

41. The method of claim 40, wherein the set of genes in step (a) consists of genes known to play role in conferring the slow burn phenotype.

42. The method of any one of claims 1-31, wherein the obesity phenotype of interest is an emotional eating phenotype.

43. The method of claim 42, wherein the specific trait is a finding or result from a behavioral questionnaire indicative of anxiety or emotional eating for the subject.

44. The method of claim 43, wherein the questionnaire is the Hospital Anxiety and Depression Scale (HADS) questionnaire.

45. The method of claim 42, wherein the plurality of genes in step (a) consists of genes known to play role in conferring the emotional eating phenotype.

46. A computer-implemented method for predicting or diagnosing a subject suffering from or suspected of suffering from obesity as possessing abnormal satiation, the method comprising:
(a) receiving in a computer system, a genetic dataset comprising a plurality of single nucleotide polymorphisms (SNPs) located in, around or near a set of genes obtained from samples obtained from each subject from a population of subjects, wherein each subject in the population of subjects is obese, wherein the set of genes comprises GLP1R, UCP2, FTO, TCF7L2, MBOAT4 (GOAT), ADRA2A, GNB3, GPBAR1, LEP, NPY1R, NPY2R, NPY4R, NPY5R, NR1H4, SLC6A4, NTS, UCP3, ADIPOQ, CCK, DPP4, FGFR4, GHRL, MC4R, MC3R, POMC, AGRP, GIPR, CNR1, FAAH, GCG, CELA2A, PPARG, TFAP2B, APOE, LEPR, SH2B1, SIM1, NCOA1 (SRC1), PCSK1, TMEM18, NEGR1, BDNF, GPRC5B, GNPDA2, MTCH2, KCTD15, SEC16B, FAIM2, TNNI3K, LINGO1, FANCL, CADM2, PTBP2, NUDT3, OLFM4, HOXB5, TNFRSF11A and DYRK1B;
(b) generating by the computer system, a contribution factor for each SNP located in or near a gene from the set of genes from the genetic dataset for a subject from the population of subjects, wherein the contribution factor is a numerical value, vector, matrix, or function of various biological factors that represents the contribution or effect that each SNP has on total kcal consumed to satiation (CTS) at an ad libitum meal;
(c) calculating by the computer system, a gene risk score (GRS) for each gene in the set of genes by integrating the contribution factors generated in step (b) for each SNP for each gene from the set of genes and SNP genotype information located in, around or near each gene in the set of genes, wherein the GRS for each gene represents the cumulative effect of the variation in SNPs located in, around, or near the gene in that particular subject has on the function or regulation of the gene so as to increase or decrease the risk of being above a sex-specific threshold total kcal consumed to satiation (CTS) at an ad libitum meal;
(d) iteratively performing by the computer system, steps (a)-(c) for each additional subject from the population of subjects to generate a set of GRSs for each gene in the set of genes, wherein each gene has a GRS for each sex; and
(c) integrating by the computer system, the GRSs from step (d) with non-genetic and/or multi-omic data obtained from the samples obtained from each subject from the population of subjects using a machine learning model to calculate a machine learning assisted gene risk score (ML-GRS), wherein the ML-GRS predicts the CTS by combining gene risk scores and other biological, psychological, or environmental measurements of the subject;
(f) training by the computer system, the ML-GRS from step (e) using a classifier model to predict subjects from a training set of samples from a training population of obese subjects as being positive for being above a sex-specific threshold total kcal consumed to satiation (CTS) at an ad libitum meal, thereby generating a trained ML-GRS; and
(g) determining by the computer system, if a test subject is positive for the specific trait known to be associated with the obesity phenotype of interest by:
(i) calculating by the computer system the ML-GRS for the test subject using steps (a)-(e) on a sample obtained from the test subject; and
(ii) comparing by the computer system, the ML-GRS for the test subject to the trained ML-GRS, wherein the test subject is positive for CTS if the ML-GRS for the subject is above the sex-specific 75^{th} percentile for CTS or negative for CTS if the ML-GRS for the subject is below the sex-specific 75^{th} percentile for CTS.

47. The method of claim 46, wherein the sample is selected from the group consisting of a blood sample, a saliva sample, a urine sample, a breath sample, and a stool sample.

48. A method for treating obesity in a subject in need thereof, the method comprising:
a. determining if the subject possesses abnormal satiation using the method of claim 46 on a genetic dataset obtained from a sample obtained the subject to determine a ML-GRS for abnormal satiation; and
b. administering a pharmacotherapy to the subject that does not include a GLP-1 agonist if the ML-GRS determined for the subject indicates that the subject is positive for CTS or administering a pharmacotherapy that may include a GLP-1 agonist to the subject if the ML-GRS determined for the subject indicates that the subject is negative for CTS.

49. A method for treating obesity in a subject in need thereof, the method comprising:
a. determining the presence, absence or level of a plurality of gastrointestinal (GI) peptides, a plurality of metabolites, and/or a plurality of genetic variants in a sample obtained from a subject suffering from obesity as well as satiety, satiation, resting energy expenditure and results on a behavioral questionnaire for the subject, thereby generating an obesity analyte signature for the sample;
b. populating a predictive machine learning model with the obesity analyte signature for the subject; and
c. utilizing the predictive machine learning model to predict an obesity phenotype of the subject suffering from obesity, wherein the obesity phenotype is selected from the group consisting of hungry brain (abnormal satiation), hungry gut (abnormal satiety), slow burn (slow metabolism) or hedonic/emotional eating); and
d. administering a pharmacotherapy to the subject that does not include a GLP-1 agonist if the predictive machine learning model to predicts that the subject possesses an abnormal satiation phenotype or administering a pharmacotherapy that may include a GLP-1 agonist to the subject if the predictive machine learning model to predicts that the subject does not possess an abnormal satiation phenotype.

50. The method of claim 48 or 49, wherein the pharmacotherapy that does not include a GLP-1 agonist is phentermine/topiramate.

51. The method of claim 48 or 49, wherein the pharmacotherapy that does include a GLP-1 agonist is selected from the group consisting of exenatide, liraglutide, albiglutide, dulaglutide, lixisenatide, semaglutide and tirzepatide.

52. A method of treating obesity in a subject in need thereof, the method comprising:
a. detecting an obesity analyte signature in a sample obtained from a subject;
wherein the obesity analyte signature is indicative of an obesity phenotype of the subject; and
b. administering a pharmacotherapy to the subject that does not include a GLP-1 agonist if the obesity analyte signature detected in the sample obtained from the subject indicates that the subject possesses an abnormal satiation phenotype or administering a pharmacotherapy that may include a GLP-1 agonist to the subject if the obesity analyte signature detected in the sample obtained from the subject indicates that the subject does not possess an abnormal satiation phenotype.

53. The method of claim 52, wherein the GLP-1 agonist is selected from the group consisting of exenatide, liraglutide, albiglutide, dulaglutide, lixisenatide, semaglutide and tirzepatide.

54. A method of identifying a subject suffering from obesity as a non-responder to treatment with a GLP-1 agonist, the method comprising detecting an obesity phenotype of the subject; and identifying the subject as a non-responder to GLP-1 agonist treatment if the subject is determined to possess an abnormal satiation obesity phenotype.

55. The method of any of one of claims 49-53, wherein the sample is selected from the group consisting of a blood sample, a saliva sample, a urine sample, a breath sample, and a stool sample.

56. The method of any one of claims 52-55, wherein the obesity analyte signature comprises the presence of serotonin, glutamine, isocaproic acid, allo-isoleucine, hydroxyproline, beta-aminoisobutyric-acid, alanine, hexanoic acid, tyrosine, and PYY, an absence of 1-methylhistine, gamma-amino-n-butyric-acid, phenylalanine, ghrelin, and a HADS questionnaire result that does not indicate an anxiety subscale (HADS-A;
e.g., includes a HADS-A questionnaire result).

57. The method of claim 56, wherein the subject consumes above the seventy-fifth percentile amount of calories at an ad libitum meal.

58. The method of any one of claims 52-55, wherein the obesity analyte signature comprises the presence of 1-methylhistine, allo-isoleucine, hydroxyproline, beta-aminoisobutyric-acid, alanine, and phenylalanine, an absence of serotonin, glutamine, gamma-amino-n-butyric-acid, isocaproic acid, hexanoic acid, tyrosine, ghrelin, PYY, and does not include a HADS questionnaire result that indicates an anxiety subscale.

59. The method of claim 58, wherein the subject has increased or accelerated baseline gastric emptying as compared to a control subject, wherein the control subject is not obese.

60. The method of any one of claims 46-49, wherein the obesity analyte signature comprises the presence of 1-methylhistine, serotonin, glutamine, gamma-amino-n-butyric-acid, isocaproic acid, allo-isoleucine, alanine, tyrosine, ghrelin, PYY, an absence of hydroxyproline, beta-aminoisobutyric-acid, hexanoic acid, and phenylalanine, and does includes a HADS questionnaire result that indicates an anxiety subscale.

61. The method of claim 52, wherein the subject has decreased or low resting energy expenditure (REE) for the subject as compared to a control, wherein the control subject is not obese.

62. The method of any one of claims 52-55, wherein the obesity analyte signature comprises the presence of serotonin, an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and does includes a HADS questionnaire result that indicates an anxiety subscale.
